(19) United States
(12) Patent Application Publication
(10) Pub. No.: US 2024/0376548 A1
(43) Pub. Date: Nov. 14, 2024

(54) EXHALED BREATH CONDENSATE CONTAINS EXTRACELLULAR VESICLES (EVS) WITH MIRNA CARGOS OF LUNG TISSUE ORIGIN THAT CAN BE SELECTIVELY PURIFIED AND ANALYZED

(71) Applicant: HACKENSACK MERIDIAN HEALTH, INC., Edison, NJ (US)

(72) Inventors: Olivier Loudig, Yorktown Heights, NY (US); Megan Irvette Mitchell, Clifton, NJ (US)

(73) Assignee: HACKENSACK MERIDIAN HEALTH, INC., Edison, NJ (US)

(21) Appl. No.: 18/661,353

(22) Filed: May 10, 2024

Related U.S. Application Data
(60) Provisional application No. 63/465,467, filed on May 10, 2023.
(60) Provisional application No. 63/550,764, filed on Feb. 7, 2024.

(51) Int. Cl. C12Q 1/6886; A61B 10/00; C12Q 1/6804; C12Q 1/6874

(52) U.S. Cl. CPC C12Q 1/6886; A61B 10/00; C12Q 1/6804; C12Q 1/6874; A61B 2010/0087; C12Q 2600/158; C12Q 2600/178

(57) ABSTRACT

The present disclosure provides a non-invasive method for early detection of deep lung pathology in a subject at risk of the deep lung pathology. The method comprises (a) sampling deep lung tissue of the subject at risk of the deep lung pathology by isolating micro RNA (riRNA) cargo of lung tissue origin in exhaled extracellular vesicles (EVs) purified from exhaled breath condensates (EBCs) of the subject; (b) purifying lung-specific exhaled extracellular vesicles in exhaled breath condensate by antibody capture of the lung-specific exhaled EVs; and (c) detecting the pathology by comparing an miRNA profile of the exhaled extracellular vesicles of lung tissue origin purified from the subject to the profile of exhaled extracellular vesicles purified from exhaled breath condensates of a healthy subject. The early detection can lead to early treatment of the pathology and an improved health outcome for the subject.

### CROSS-REFERENCE TO RELATED APPLICATIONS

This application claims priority from U.S. provisional 63/465,467 (filed 10 May 2023) and from U.S. provisional 63/550,764 (filed 7 Feb. 2024, the content of each of these applications is incorporated by reference in its entirety.

### FIELD OF THE INVENTION

The present invention relates to non-invvasive selective purification and analysis of extracellular vesicles of lung tissue origin from exhaled breath condensates for the discovery of exhaled biomarkers and the detection of deep lung pathologies.

### BACKGROUND OF THE INVENTION

Pulmonary disease is ranked as the third-leading cause of mortality worldwide [1]. Chronic obstructive pulmonary disease (COPD) and asthma, respectively claimed the lives of 3.3 and 0.4 million people worldwide in 2019 [2,3]. Lung cancer alone has steadily been ranked as the second leading cause of cancer incidence (2.1 million cases per year) and mortality (1.8 million deaths per year) in both men and women worldwide [4-6]. It is estimated that the number of people diagnosed with lung cancer and those who die from it will nearly double by 2040 [7,8]. However, contrary to other pulmonary diseases, early-stage lung cancer presents with common and nonspecific respiratory symptoms, which can delay its detection and contribute to poor outcomes. Although there is a direct correlation between smoking and the development of lung tumors, only a fraction of high-risk subjects actively engages in early screening programs [9,10]. Additionally, even though smoking is a major known risk factor, other environmental and genetic factors, which remain largely unknown, make early identification of individuals who will develop lung cancer difficult. Currently, it is estimated that only ˜15% of new lung cancer cases are diagnosed at an early and curable stage [11-13]. Thus, there is an urgent need for the development of noninvasive, rapid, cost-effective and highly sensitive early detection strategies to improve disease outcome.

Although computed tomography (CT) scan is widely used to detect lung abnormalities, it cannot accurately distinguish between benign and malignant lung nodules, and for a complete clinical diagnosis invasive biopsies are required [14-16]. As an alternative non-invasive approach, it has been proposed that targeted molecular testing may achieve a greater sensitivity than current clinical imaging modalities [17]. Therefore, several molecular assays have been evaluated for the ultra-sensitive detection of circulating tumor material, including circulating tumor cells (CTCs), circulating tumor DNA (ctDNA), deregulated circulating transcripts (circular RNAs, mRNAs, microRNAs (miRNAs), long non-coding RNAs), and more recently circulating biomarkers (DNA, RNAs, proteins) stably packaged within circulating tumor extracellular vesicles [18-21]. Although these technological advances may help decrease the invasiveness of testing, the sensitivity of blood-based biomarkers remains limited to the detection of advanced lung cancer.

Considering that the lung communicates directly with the external environment, different types of airway-derived biofluids (i.e., saliva, sputum, bronchial brushing, bronchoalveolar lavage (BAL), etc.,) have been experimentally evaluated for their potential to provide meaningful biomarkers originating from the lung and to help develop more accurate lung cancer detection strategies [22-32]. Although bronchoscopy with BAL is the most direct approach for sampling the biofluid that lines the lower respiratory tract where lung tumors reside, the invasive nature of this procedure has precluded its implementation as a screening approach and has ignited a search for less intrusive airway sampling methods. Of these the collection of exhaled breath has gained a lot of interest because it is minimally invasive and can simply be carried out during normal tidal respiration [33,34]. Biochemical analyses of exhaled breath have demonstrated that it contains two distinct biological fractions, which can be separately collected and analyzed: volatile organic compounds (VOCs) which are gases diffused from the circulation, and non-volatile organic compounds (non-VOCs), which are aerosolized compounds originating from the respiratory tract [35]. Non-VOCs can easily be condensed into a biofluid also known as exhaled breath condensate (EBC), which is of interest in molecular biology because it contains metabolites (e.g., nitrite, urea, amino acid) and macromolecules (i.e., proteins, RNA, DNA) that can be precisely quantified even after long term storage whereas VOCs can only be analyzed during subject monitoring [32,36,37].

The collection of EBC can be performed non-invasively using FDA-registered commercial devices [35,38,39]. It is thought that the macromolecules contained in EBC become aerosolized from the biofluid that lines epithelial lung cells due to the velocity of air turbulences caused by normal respiration inside the lungs [35]. Early analyses of EBC content have revealed the presence of surfactant proteins, which suggested that some of the macromolecules may originate in deep lung alveoli, the respiratory units responsible for gas exchanges between the circulatory system and the external environment [40]. The terminal bronchioles are the smallest airway passages in direct contact with the alveoli where different types of epithelial cells reside, including the non-ciliated secretory lung-specific Clara cells [41]. These cells are particularly interesting because they uniquely express the Clara Cell Specific Protein (CCSP) in the lung tissue and secrete it in large quantities to protect the respiratory tract against oxidative stress and inflammation [42]. Contrarily to the terminal bronchioles, the alveoli are only composed of two types of cells, the alveolar type I cells that are involved in gas exchange and the alveolar type II cells that are progenitors of type I cells but that produce all pulmonary surfactant, whose purpose is to reduce the air-liquid surface tension to enhance gas exchange and to actively contribute to the immune defense of the lungs [43]. Surfactant Protein C (SFTPC) is one of these molecules and it is uniquely produced by alveolar type II cells [44,45]. Although the exact cellular origin of lung cancer initiating cells remain unknown, both Clara cells and alveolar type II cells have been experimentally implicated in the initiation of lung adenocarcinoma [46-48].

To date, several molecular studies conducted on EBC have been focused on its DNA and RNA content with genomic DNA methylation profiling [49], genomic DNA mutational profiling [50], mitochondrial DNA mutational profiling [51], the profiling of uniquely deregulated mRNAs [52], and more recently the profiling of microRNAs (miRNAs) to evaluate the detection of lung cancer, including a study that we recently conducted [53-56]. MiRNAs are of particular interest because their deregulated expression is directly associated with the initiation and development of lung cancers [49-51], and they have shown promise for prognostic evaluation of lung cancers [57-59], and their quantitation in biofluids shows promise for prognostic evaluation of lung tumors [60-62]. However, considering that intact exhaled miRNAs can be detected in EBC [53], we and others have hypothesized that they may be packaged and protected within exhaled Extracellular Vesicles (exh-EVs) [63-67]. Studies have shown that the molecular cargos of EVs are protected from degradation by a robust double-layered membrane, which harbors generic (i.e., common to many cell types) and cell-specific surface proteins and receptors acquired from their cell-of-origin [68,69]. EVs are produced by all human cells and due to their small size (˜30 to 120 nm in diameter) they can diffuse into tissues and circulate in any biofluid [68,69]. Particularly, recent studies have demonstrated that EVs released by tumor cells contain a variety of pre-packaged functional biomolecules including miRNAs, whose local and long-distance delivery to target cells enables intercellular communication [70,71]. Functional studies of EVs produced released in different biofluids (i.e., blood, BAL, and pleural lavage) by primary lung tumor cells [72] have demonstrated that their miRNA cargos, which are differentially packaged than those of normal cells, can modulate angiogenesis [73], cellular proliferation [74], and immune response of target cells [75] and also participate in the educate distal pre-metastatic lung niche cells to promote the uptake of CTCs [76]. Thus, it is currently proposed that the purification of tumor EVs from airway biofluids and the quantification of their miRNA cargos may enable non-invasive detection of unique signatures produced by tumor cells for non-invasive detection of lung cancer.

Considering that only a few studies have suggested that EBC may contains EVs [77-79], our primary objective was to experimentally confirm the presence and precisely validate the identity of EVs contained in human EBC. Upon purification and identification of exhaled EVs (exh-EVs), we sought to determine whether they contained miRNAs of lung tissue origin and thus conducted small-RNA next generation sequencing (NGS) analyses of EBC and exh-EVs in comparison to other airway biofluids. Next, by leveraging our ultra-sensitive and customizable EV-CATCHER (Extracellular Vesicle Capture by AnTibody of CHoice and Enzymatic Release) purification assay [80] to selectively target different populations of exh-EVs, we evaluated the potential of uniquely selected exh-EV miRNA cargos of lung tissue origin to distinguish subjects based on the condition of their respiratory tract.

### SUMMARY OF THE INVENTION

### Brief Description of the Drawings

The patent or application file contains at least one drawing executed in color. Copies of this patent or patent application publication with color drawing(s) will be provided by the Office upon request and payment of the necessary fee.

FIG. 1A, FIG. 1B, FIG. 1C illustrate the airway system and the non-invasive collection of exhaled breath condensates (EBC). FIG. 1A shows that volatile organic compounds (VOCs) and non-volatile organic compounds (non-VOCs) are exhaled out of the lungs during normal tidal respiration. Non-VOCs are enriched in metabolites and macromolecules by aerosolization of biofluids from the respiratory tract. FIG. 1B shows the collection of exhaled breath condensates using the disposable RTube™ device (FDA-registered). Prior to collection of EBC a pre-frozen metal cylinder, housed within an insulated cloth sleeve, is applied over the polypropylene section of the RTube™ collection device. The volunteer subject uses the mouthpiece of the device to inhale (see blue arrow for inhaled air) and exhale (see red arrow for exhaled air) during normal tidal respiration, for a period of 10-minutes. The exhaled air, which contains moisture, is progressively condensed into a biofluid onto the non-absorbent surface of the transparent polypropylene collection tube. Upon completion of the EBC collection, the transparent collection tube is disconnected, positioned onto a plunger where the lower gasket is pushed upward to gather the biofluid at the top of the tube. The EBC is then collected with a pipette and transferred into an Eppendorf for storage or analysis. FIG. 1C shows an architectural cellular representation of the terminal bronchiole and alveoli unit with its different epithelial cells. The terminal bronchiole is comprised of Ciliated, Goblet, Clara and Basal epithelial cells. Clara cell uniquely express the Clara Cell Secretory protein (CCSP). The alveoli are composed of Alveolar Epithelial Type I (ATI) and Alveolar Epithelial Type II cells (ATII). ATII cells uniquely express different surfactant proteins and particularly Surfactant Protein C, which is a membrane protein that undergoes cleavage before release into the biofluid lining the lungs.

FIG. 2A, FIG. 2B, FIG. 2C, and FIG. 2D illustrate identification and validation of exhaled Extracellular Vesicles (exh-EVs). FIG. 2A. Image of the Spectradyne nCS1 nanoparticle analyzer, C400 cartridges used to quantify nanoparticles between 65-400 nm, and the schematic of the Resistive Pulse Sensing mechanical process employed to count and size nanoparticles. EBC samples collected with the RTube™ device from four healthy subjects (See green, red, light blue, and purple plots) were subjected to the human anti-CD63 EV-CATCHER assay and were analyzed on a Spectradyne nCS1 nanoparticle analysis instrument. The size of the nanoparticles detected in these purified samples is displayed on the x-axis of the plot (nanometers), and their number on the y-axis of the plot (particle per milliliter per nanometer). The dark blue plot displays a negative control of the EV-CATCHER assay performed using 1×PBS to account for any signal obtained from the digested Ab-DNA. FIG. 2B. Schematics of ultracentrifugation (UC) and the tTSP (CD9/CD63/CD81) EV-CATCHER assay, used to purify extracellular vesicles (EVs) from the EBC used to purify exh-EVs. FIG. 2C (top) schematically shows customization of the EV-CATCHER assay with anti-CCSP and anti-SFTPC antibodies and purified EVs from ultracentrifuged pellets from two EBC samples (i.e., healthy volunteers) from Subject #1 and Subject #2. FIG. 2C (bottom) shows Transmission Electron Microscopy (TEM) images of exh-EVs purified from the EBC from the two controls after ultracentrifugation (left column), tTSP EV-CATCHER (middle) and anti CCSP/5FTPC EV-CATHER (right column). FIG. 2D is a schematic of the different exh-EV proteins detected by our fluorescent antibodies. Representative ONi super resolution nanoimaging images of exh-EVs purified from the EBC of our two healthy controls by UC, tTSP (CD9/CD63/CD81) EV-CATCHER, and the CCSP/SFTPC EV-CATCHER assays. Antibodies targeted against CD9-AlexaFluor™ 488 (cyan), CD63-AlexaFluor™ 568 (yellow) and either Clara Cell Secretory Protein (CCSP)-AlexaFluor™ 647 (purple) or the Surfactant Protein C (SFTPC)-AlexaFluor™ 647 (red) allowed for detection of their surface expression of immobilized exh-EVs.

FIG. 3A, FIG. 3B, and FIG. 3C illustrate comparative microRNA expression analyses of samples collected from five different anatomic airway levels from the same 18 subjects. FIG. 3A shows a schematic representation for the collection and analysis of 5 level of airway specimens including mouth rinse, mouth brushing, bronchial brushing, bronchoalveolar lavage (BAL) and EBC from the same 18 subjects. Small-RNA collections were preformed from whole mouth rinse, from the 1×PBS solution for the brush used for buccal brushing, and the 1×PBS solution for the brush used for bronchial brushing. For EBC and BAL, small-RNA was extracted from whole biofluids, and from exh-EVs purified from both biofluids with our tTSP EVCATCHER. FIG. 3B shows heat map analysis of small-RNA expression profiles obtained by next generation sequencing (NGS) of the RNA extracted from the 5 levels of airway sampling. The four rows of rectangles above the heatmap indicate: 1^{st} row for airway origin (mouth rinse (red), buccal brushing (purple), bronchial brushing (yellow), BAL (green), and EBC (blue)), 2nd row for the type of selection (whole biofluid or tTSP EV-CATCHER purification of exh-EVs), 3rd row for the donors ID (from 18 subjects), and 4^{th} row for the level of miRNA expression (log 2 of the total miRNA reads). The top box plot of FIG. 3C displays the total miRNA read distribution for all 18 donors (NGS small-RNA libraries 1-7; See Table 1) for the 5 airway levels of collection (mouth rinse, buccal brush, bronchial brush, bronchoalveolar lavage (BAL), and EBC). The bottom left box plot of FIG. 3C displays the miRNA distribution for all 18 donors for small-RNA extracted and sequenced from whole BAL (left) and BAL EVs (right) purified with the tTSP EV-CATCHER assay from the same BAL samples. The bottom right box plot of FIG. 3C displays the miRNA distribution for all 18 donors for small-RNA extracted and sequenced from whole EBC and exh-EVs purified with the tTSP EV-CATCHER assay from the same EBC samples.

FIG. 4A, FIG. 4B and FIG. 4C illustrate a comparison of total miRNA reads and small-RNA species distribution based on airway level and purification methods. FIG. 4A is a diagram displaying the small-RNA NGS scheme for specimens collected from 69 subjects (see Table 1 (n=18) and Supplemental Table 1 (n=51)). In FIG. 4B, for libraries 1 to 7, miRNA expression profiling was conducted on 5 different levels of airway (i.e., Mouth rinse (MR), Buccal brush (BB), Bronchial brush (BrB), Bronchoalveolar lavage (BAL), and EBC) and EVs isolated using the tTSP EV-CATCHER assay from BAL and EBC, from the same 18 subjects (see Table 1, subjects 1 to 18). Small-RNA libraries 8-11 (See Supplemental Table 1, n=15; subjects 19 to 33) were prepared to analyze the miRNA expression profiles of subject-matched BAL (whole and tTSP EV-CATCHER purified EVs) and EBC (whole and tTSP EV-CATCHER purified EVs) samples. Small-RNA libraries 12-17 (See Supplemental Table 1, n=18; subjects 34 to 51) were prepared to analyze the miRNA expression profiles of subject-matched BAL (whole, tTSP EV-CATCHER, and anti-CCSP/SFTPC EV-CATCHER purified EVs), and EBC (whole, tTSP EV-CATCHER, and anti-CCSP/SFTPC EV-CATCHER purified EVs) samples. Small-RNA libraries 18-21 (See Supplemental Table 1, n=18; subjects 52 to 69) were prepared using small-RNA extracted from EVs purified with the tTSP EV-CATCHER and the anti-CCSP/SFTPC EV-CATCHER assays from subject-matched BAL and EBC samples. FIG. 4C shows read distribution and expression differences for small-RNAs extracted and sequenced for libraries 12 to 17, from whole EBC (dark blue box plots), exh-EVs purified with the tTSP EV-CATCHER assay (medium blue box plots) and exh-EVs purified with the anti-CCSP/SFTPC EV-CATCHER assay (light blue box plots) from the same 18 EBC samples (See Supplemental Table 1, n=18; subjects 34 to 51).

FIG. 5A, FIG. 5B, FIG. 5C, and FIG. 5D provide insights from miRNA expression analyses of exh-EVs as a surrogate measure for BAL EVs, highlighting distinctions in subjects based on smoking, asthma, or tumor status. FIG. 5A is a schematic illustrating the careful selection of matched BAL and EBC samples from subjects, based on their smoking, asthma, and tumor status, for subsequent miRNA NGS analyses. FIG. 5B shows that among the top 317 miRNAs consistently detected in BAL and EBC (libraries 12-17 and 18-21; Supplemental Table 1), with known smoking statuses (never (n=13), former (n=13), current (n=10)), box plot comparative analyses showcase miRNA z-scores. Calculations were performed on matched BAL and EBC samples for small-RNA analyses encompassing whole BAL and whole EBC (top and bottom left panels), BAL EVs and exh-EVs purified with the tTSP EV-CATCHER assay (top and bottom center panels), and BAL EVs and exh-EVs purified with the anti-CCSP/SFTPC EV-CATCHER assay (top and bottom right panels). The miRNA z-scores amalgamate the top upregulated miRNAs (n=8) and top downregulated miRNAs (n=7). P-values for miRNA-derived z-scores were determined using pairwise t-tests between never and current smokers, presented in each plot. FIG. 5C is a box plot representation of miRNA z-scores for matched BAL and EBC specimens (subjects #52-69; libraries 1-21; Table 1) with EVs purified using the tTSP and anti-CCSP/SFTPC EV-CATCHER assays. This analysis contrasts control subjects without asthma (n=15) with subjects having asthma (n=13). MiRNA z-scores reflect a combination of the top upregulated miRNAs (n=4) and top downregulated miRNAs (n=7), commonly identified in RNA purified from EVs obtained through the tTSP EV-CATCHER assay for matched BAL and EBC samples. P-values were calculated using pairwise t-tests. FIG. 5D is a box plot representation of miRNA z-scores for matched BAL and EBC specimens (subjects #52-69; libraries 12-21; Supplemental Table 1) with EVs purified using the tTSP and anti-CCSP/SFTPC EV-CATCHER assays. These comparative analyses focus on BAL and EBC samples from 7 individuals with benign lung lesions and 9 patients diagnosed with lung adenocarcinoma (selected from libraries 12-21; Supplemental Table 1). MiRNA z-scores are computed by summing the z-values of the two most upregulated (miR-126-3p, miR-339-3p) and two most downregulated miRNAs (let-7c, miR-184) in samples from patients with adenocarcinoma compared to those without cancer. P-values were calculated using pairwise t-tests.

FIG. 6A, FIG. 6B, FIG. 6C, FIG. 6D and FIG. 6E illustrate miRNA expression comparative analyses between exh-EVs purified with the tTSP and the anti-CCSP/SFTPC EV-CATCHER assays from a new set of EBC-only samples. FIG. 6A. The human figure displays that EBC-only samples were collected from 18 subjects for evaluation of their tumor status using our two different exh-EV purification methods prior to miRNA NGS analyses. FIG. 6B shows principal Component Analysis (PCA) plots that display the similarities in global miRNA expression between the 12 control (Healthy) and the 6 cancer (Lung cancer) subjects for exh-EVs that were purified from their EBC with the tTSP EV-CATCHER assay and the anti-CCSP/SFTPC EV-CATCHER assay. FIG. 6C shows heat map analysis of the top 19 most differentially expressed miRNAs between the 12 controls (Healthy) and the 6 cancer (Lung cancer) subjects for exh-EVs that were purified from EBC using with the tTSP EV-CATCHER assay or the anti-CCSP/SFTPC EV-CATCHER assay. FIG. 6D shows box plot analyses of the top 19 most differentially expressed miRNAs between the 12 controls (Healthy) and the 6 cancer (Lung cancer) subjects, displaying significant log 2 fold expression differences, as detected by small-RNA NGS analyses. FIG. 6E shows box plot analyses of the z-score obtained from the top 14 (out of 19) most differentially expressed miRNAs quantified by small-RNA NGS analyses between the 12 controls (healthy) and the 6 cancer (lung cancer) subjects for exh-EVs purified with the tTSP EV-CATCHER assay (left) or the anti-CCSP/SFTPC EV-CATCHER assay (right). The p-values identified between healthy controls and patients diagnosed with advanced lung cancer are displayed in the two panels.

FIG. 7. The equipment and RTube™ device required for EBC collection are detailed in the upper section of the figure in blue. From left to right, the pre-packaged RTube™ contained in a sterile plastic bag (1), which is opened to reveal the full device (2). The aluminum cylinder that is frozen (3), the cloth sleeve for the aluminum cylinder (4), and the aluminum plunger (5) that is also use as a stand for the aluminum cylinder when it is stored at −20° C. Then, the process by which EBC is condensed onto the inner surface of the polypropylene tube is displayed in the middle section of the figure in light green. From left to right, the aluminum cylinder with its cloth sleeve is stored onto the plunger (6), or it is placed directly onto the RTube™ prior to the collection of EBC samples (7). After 5 minutes of EBC collection the aluminum cylinder is removed (8) to highlight the droplets that are formed onto the inner surface of the polypropylene RTube™ (9). After 10 minutes of collection, droplets are uniformly formed in the inner surface of the polypropylene RTube™ (10), at the top (11), and some of the condensed droplets have accumulated around the breathing valve (12). Finally, we display the process EBC condensate is collected and transferred to an Eppendorf is detailed in the lower section of the figure in dark green. From left to right, the polypropylene RTube™ is detached from the mouth piece and placed onto the plunger (13). As the valve is pushed upward, liquid forms around the valve (14). When the valve has been pushed all the way up on the plunger (15), the EBC sample is fully accumulated round the breathing valve (15). Using a 1 ml pipette (16), the EBC sample is collected and transferred right away into an Eppendorf (17) and stored at −80° C. for long term storage.

FIG. 8 shows ONi super resolution nanoimaging whole field images of the analyses conducted and displayed in FIG. 2D. Representative wide view images of ONi super resolution microscopy imaging fields of exh-EVs purified from the EBC from two healthy controls by UC, the tTSP EV-CATCHER assay, and the anti-CCSP/SFTPC EV-CATCHER assay. Antibodies targeted against CD9-AlexaFluor™ 488, CD63-AlexaFluor™ 568 and either Clara Cell Secretory Protein (CCSP)-AlexaFluor™ 647 or Surfactant Protein C (SFTPC)-AlexaFluor™ 647 were used to detect proteins on the surface of exh-EVs. Using the CODI online processing software from ONi raw images were processed using the EV profiling application. Images represent wide fields of view following DBSCAN clustering algorithm constraining clusters to only highlight (white) triple positive EVs with a minimum cluster density of 10 and a minimum size of 20 and excluding (grey) all other EVs.”

FIG. 9 illustrates validation of EVs detected in Bronchoalveolar lavage (BAL). FIG. 9A shows an image of the Spectradyne nCS1 nanoparticle analyzer, the C-400 cartridge used to quantify nanoparticles between 65-400 nm, and the schematic of the Resistive Pulse Sensing mechanical process employed to count and size nanoparticles. BAL samples collected from two subjects (1 ml from each) recruited at the Montefiore Medical Center (See subject IDs matching detailed in Supplemental Table 1) from two subjects were subjected to ultracentrifugation and 5 ml of their resuspended pellets (i.e., in 1×PBS) were analyzed on the Spectradyne nCS1 instrument. The dark blue plot displays detection of nanoparticles in BAL of a subject without known lung disease. The green plot displays nanoparticles detected in BAL of a subject diagnosed with asthma. FIG. 9B shows schematics of ultracentrifugation (UC) and Transmission Electron Microscopy (TEM) of ultracentrifuged BAL pellets from the same two subjects. The images display detection of membrane-bound extracellular vesicles with sizes ranging between 100-200 nm and displaying the abundance of EVs found in 1 ml BAL. FIG. 9C shows ONi of EVs purified by ultracentrifugation from BAL of two subjects.

### DETAILED DESCRIPTION OF THE INVENTION

### Definitions

As used herein and in the appended claims, the singular forms “a” “an”, and “the” include plural reference unless the context clearly dictates otherwise. Thus, for example, reference to a “peptide” is a reference to one or more peptides and equivalents thereof known to those skilled in the art, and so forth.

The term “alveoli” (singular, alveolus) as used herein refer to microscopic balloon-shaped air sacs located at the end of the respiratory tree. They expand during inhalation, taking in oxygen, and shrink during exhalation, expelling carbon dioxide. Gas exchange in the lung occurs within alveoli composed of type 2 and type 1 epithelial cells (AEC2s and AEC1s), capillaries, and various resident mesenchymal cells. Surfactant protein C-positive (SFTPC-positive) AEC2s self renew and differentiate over about a year, consistent with the population containing long-term alveolar stem cells. Genetic lineage-tracing studies in the mouse established that SFTPC+ AEC2s, as a population, proliferate in vivo and give rise to AEC1s [Barkauskas, C E et al. J. Clin. Invest. (2013) 123 (7): 3025-36].

The terms “animal,” “patient,” and “subject” as used herein include, but are not limited to, humans and non-human vertebrates such as wild, domestic, and farm animals. The terms “animal,” “patient,” and “subject” may refer to mammals, including humans.

The term “antibody” as used herein refers to a polypeptide or group of polypeptides comprised of at least one binding domain that is formed from the folding of polypeptide chains having three-dimensional binding spaces with internal surface shapes and charge distributions complementary to the features of an antigenic determinant of an antigen.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface. Human antibodies show two kinds of light chains, κ and λ; individual molecules of immunoglobulin generally are only one or the other.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques. An antibody may be from any species.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma.

The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety.

As used herein, the terms “antigen” refers to any substance that elicits an immune response.

The term “antigen-binding site” as used herein refers to the site at the tip of each arm of an antibody that makes physical contact with an antigen and binds it noncovalently. The antigen specificity of the antigen-binding site is determined by its shape and the amino acids present.

The term “antigenic determinant” or “epitope” as used herein refers to that portion of an antigenic molecule that is contacted by the antigen-binding site of a given antibody or antigen receptor.

The terms “residue” or “amino acid residue” or “amino acid” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

The term “binding” and its other grammatical forms as used herein means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

As used herein, the term “binding agent” refer to a substance that can bind to a chemical or other substance, e.g., an antigen.

As used herein, the term “biological particle” refers to a minute portion, piece, fragment or amount (particle) derived from an organism. Biological particles include, without limitation, exosomes, extracellular vesicles, viral particles, bacterial particles, or other secreted particles comprising surface membranes.

The term “biomarker” (or “biosignature”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes).

A “predictive biomarker” is a biomolecule that indicates therapeutic efficacy, i.e., an interaction exists between the biomolecule and therapy that impacts patient outcome.

A “prognostic biomarker”, which is an indicator of innate tumor aggressiveness, is a biomolecule that indicates patient survival independent of the treatment received.

The term “breathing” as used herein refers to the process that moves air in (inhalation) and out exhalation) of the lungs.

The term “bronchial brushing” as sued herein refers to a biopsy procedure used to find cancer and changes in cells that may lead to cancer. A bronchoscope (a thin, tube-like instrument with a light and a lens for viewing) is inserted through the nose or mouth into the lungs. A small brush is then used to remove cells from the airways that lead to the lungs. These cells are then examined under a microscope.

The term “bronchoalveolar lavage” (BAL) is used herein to refer to a medical procedure in which a bronchoscope is passed through the mouth or nose into the lungs and fluid is squirted into a small part of the lung and then collected for examination. “Bronchoalveolar lavage fluid” (BALF) is used herein to refer to the fluid collected from a BAL procedure.

The term “cancer” as used herein refers to diseases in which abnormal cells divide without control and can invade nearby tissues. Cancer cells can also spread to other parts of the body through the blood and lymph systems.

The term “carcinoma” as used herein refers to a cancer that begins in the skin or in tissues that line or cover internal organs.

The term “cargo” as used herein refers to a load or that which is conveyed. With respect to exosomes and/or extracellular vesicles, the term cargo refers to a substance encapsulated in the exosome and/or extracellular vesicle. The compound or substance can be, e.g., a nucleic acid (e.g., nucleotides, DNA, RNA), a polypeptide, a lipid, a protein, or a metabolite, or any other substance that can be encapsulated in an exosome and/or extracellular vesicle. With respect to exosomes and/or extracellular vesicles, the term “cargo profile” as used herein refers to the measurement of the abundance of cargo components (e.g., a nucleic acid (e.g., nucleotides, DNA, RNA), a polypeptide, a lipid, a protein, or a metabolite) that characterize the population of exosomes and/or extracellular vesicles.

The term “CD9” as used herein refers to a member of the tetraspanin protein family whose crystal structure shows a reversed cone-like molecular shape, which generates membrane curvature in the crystalline lipid layers. (Umeda, R. et al. Nature Communic. (2020) 11: article 1606).

The term “CD37” as used herein refers to a member of the tetraspanin protein family exclusively expressed on immune cells. (Zuidscherwoude, M. et al. Scientific Reports (2015) 5: 12201).

The term “CD63” as used herein refers to a member of the tetraspanin protein family, the C-terminal domain of which interacts with several subunits of adaptor protein (AP) complexes, linking the traffic of this tetraspanin to clathrin-dependent pathways (Andreu, Z. & Yanez-Mo, M., citing Rous, B A et al. Mol. Biol. Cell (2002) 13 (3): 1071-82). Among intracellular interacting proteins, CD63 was shown to directly bind to syntenin-1, a double PDZ domain-containing protein (Id., citing Latysheva, N. et al. Mol. Cell Biol. (2006) 26 (20): 7707-18). A major role in exosome biogenesis has been reported for Syntenin-1 (Id., citing Baietti, M F et al. Nat. Cell Biol. (2012) 14 (7): 677-85).

The term “CD81” as used herein refers to a member of the tetraspanin protein family whose crystal structure shows a reversed teepee-like arrangement of the four transmembrane (TM) helices, which create a central pocket in the intramembranous region that appears to bind cholesterol in the central cavity. (Zimmerman, B. et al. Cell (2016) 167: 1041-51). During development, CD81 regulates the trafficking of CD19, an essential co-stimulatory molecule of lymphoid B cells and a well-characterized CD81 partner, along the secretory pathway. (Shoham, T. et al. J. Imunol. (2003) 171: 4062-72). CD9 and CD81 have been shown to regulate several cell-cell fusion processes. (Charrin, S. et al. J. Cell Science (2014) 127: 3641-48).

The term “CD82” as used herein refers to a member of the tetraspanin protein family that has been implicated in the regulation of protein sorting into EVs and in antigen presentation by antigen presenting cells. (Andreu, Z. and Yanez-Mo, M. Front. Immunol. (2014) doi.org/10.3389/fimmu.2014.00442).

The term “Clara cell” or “Club cells” as used herein refers to non-ciliated, non-mucous, secretory cells in respiratory epithelium that secrete several distinctive proteins, including Clara cell 10-kDa secretory protein (CCSP). While the Clara cell is the principal secretory cell type throughout the tracheobronchial airways of the laboratory mouse, they are most predominant in the terminal and respiratory bronchioles of humans and monkeys. The primary functions of Clara cells are: (1) to provide secretory surfactants (surfactant proteins A, B and D) and other specific proteins (e.g., CCSP) that contribute to the airway epithelial lining fluid; (2) to serve as progenitor cells for ciliated and secretory epithelial cells; and (3) to metabolize xenobiotic compounds through P450 cytochrome-dependent mixed-function oxygenases associated with the smooth endoplasmic reticulum (SER).

The term “Clara cell secretory protein” (also known as “CCSP” or “CC16” or CC10” or “Uteroglobin) as used herein refers to an abundant protein of the airway lining fluid, which is secreted by club cells, as well as by serous and goblet cells of the proximal airways. Structurally, it is a small homodimeric protein consisting of two 8-kDa subunits aligned by disulfide links to form a hydrophobic pocket, which can bind phosphatidylcholine and phosphatidylinositol. CCSP has been noted to modulate inflammatory responses, suggesting a potential immunoregulatory role within the airways and alveoli. In vitro, CCSP has been shown to inhibit PMN chemotaxis, as well as macrophage phagocytosis. Its hydrophobic pocket allows CCSP to scavenge phospholipase A2 within the milieu, further limiting PMN activation. CCSP has also been shown to inhibit the production and bioactivity of IFN-7. [Good, M. et al. Chapter 130 in Fetal and Neonatal Physiology (5^{th} Ed.) (2017) 2: pp. 1262-93.e12. doi.org/10.1016/B978-0-323-35214-7.00130-x].

The term “click chemistry” as used herein refers to chemical synthetic methods for making compounds using reagents that can be joined together using efficient reagent conditions and that can be performed in benign solvents or solvents that can be removed or extracted using facile methods, such as evaporation, extraction, or distillation. Several types of reactions that fulfill these criteria have been identified, including nucleophilic ring opening reactions of epoxides and aziridines, non-aldol type carbonyl reactions, such as formation of hydrazones and heterocycles, additions to carbon-carbon multiple bonds, such as oxidative formation of epoxides and Michael additions, and cycloaddition reactions. A representative example of click chemistry is a reaction depicted in Formula I below that couples an azide and an alkyne to form a triazole. The copper-catalyzed azide-alkyne cycloaddition (CuAAC) features an enormous rate acceleration of 10⁷ to 10⁸ compared to the uncatalyzed 1,3-dipolar cycloaddition. It succeeds over a broad temperature range, is insensitive to aqueous conditions and pH range over 4 to 12, and tolerates a broad range of functional groups. Pure products can be isolated by simple filtration or extraction without the need for chromatography or recrystallization.

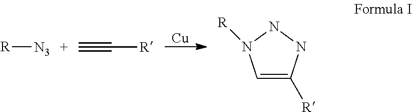

A representative example of copper-free click chemistry is a reaction that couples a dibenzocyclo-octyl (DBCO)-tagged DNA molecule to an azide-functionalized surface by cycloaddition without copper as shown in Formula II:

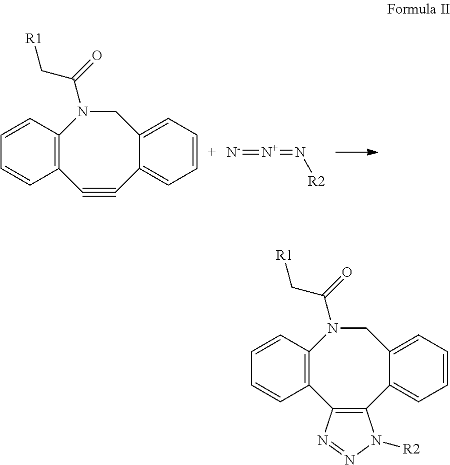

[Eeftens, J M, et al. BMC Biophys. (2015) 8: 9].

The term “clickable functional group” as used herein refers to a functional group that can be used in click chemistry to form a product. According to some embodiments, the clickable functional group is an azide or an alkyne.

The term “conjugate” as used herein refers to a compound formed by the joining or linking together of two or more chemical compounds.

The term “contact” and its various grammatical forms as used herein refers to a state or condition of touching or of immediate or local proximity.

As used herein, the term “derived from” refers to any method for receiving, obtaining, or modifying something from a source of origin.

The term “encapsulated” as used herein refers to being enclosed in a capsule (meaning a membranous envelope enclosing a part).

The term “exhalation” as used herein refers to expulsion of air from the lungs in breathing. Exhalation generally occurs passively.

The term “exhalteed breath condensate” or “EBC” as used herein is the liquid phase of exhaled air sampled by cooling, and reflects the composition of the airway lining fluid (ALF) [Karitonov, A and Barnes, P J Chest (2006) 130 (5): 1541-6].

The term “exosomes and/or extracellular vesicles” as used herein refers to extracellular bilayered membrane-bound vesicles of endosomal origin in a size range of ˜40 to 160 nm in diameter (˜100 nm on average) generated by all cells that are actively secreted.

Biogenesis. Exosomes and/or extracellular vesicles are generated in a process that involves double invagination of the plasma membrane and the formation of intracellular multivesicular bodies (MVBs) containing intraluminal vesicles (ILVs). ILVs are ultimately secreted as exosomes and/or extracellular vesicles with a size range of ˜40 to 160 nm in diameter through MVB fusion to the plasma membrane and exocytosis. The first invagination of the plasma membrane forms a cup-shaped structure that includes cell-surface proteins and soluble proteins associated with the extracellular milieu. This leads to the de novo formation of an early-sorting endosome (ESE) and in some cases may directly merge with a preexisting ESE. The trans-Golgi network and endoplasmic reticulum can also contribute to the formation and the content of the ESE (Kalluri, R., LeBleu, VS. Science (2020) 367 (6478): eaau6977, citing Kalluri, R. J. Clin. Invest. (2016) 126: 1208-15; van Neil, G. et al. Nat. Rev. Mol. Cell Biol. (2018) 19: 213-28; McAndrews, KM, Kalluri, R. Mol. Cancer (2019) 18: 52; Mathieu, M. et al. Nat. Cell Biol. (2019) 21: 9-17; Willms, E. et al. Front. Immunol. (2018) 9: 738; Hessvik, NP, Llorente, A. Cell Mol. Life Sci. (2018) 75: 193-208). ESEs can mature into late-sorting endosomes (LSEs) and eventually generate MVBs, which are also called multivesicular endosomes. MVBs form by inward invagination of the endosomal limiting membrane (that is, double invagination of the plasma membrane). This process results in MVBs containing several ILVs (future exosomes and/or extracellular vesicles). The MVB can either fuse with lysosomes or autophagosomes to be degraded or fuse with the plasma membrane to release the contained ILVs as exosomes and/or extracellular vesicles [Id., citing Kahler, C., Kalluri, R. J. Mol. Med. (2013) 91: 431037].

Heterogeneity: The heterogeneity of extracellular vesicles is thought to be reflective of their size, content, functional impact on recipient cells, and cellular origin. During their secretion they acquire surface proteins from their cell of origin. They naturally transport mRNA, miRNA, and proteins between cells.

Biomarkers. There is general agreement that their membranes are specifically enriched in tetraspanins CD9, CD37, CD63, CD81, and CD82.

Role. Extracellular vesicles are mediators of near and long-distance intercellular communication in health and disease and affect various aspects of cell biology.

As used herein, the term “expression” and its other grammatical forms refers to production of an observable phenotype by a gene, usually by directing the synthesis of a protein. It includes the biosynthesis of mRNA, polypeptide biosynthesis, polypeptide activation, e.g., by post-translational modification, or an activation of expression by changing the subcellular location or by recruitment to chromatin.

The term “extracellular vesicles (EVs)” as used herein refers to nanosized, membrane-bound vesicles released from cells that can transport cargo-including DNA, RNA, and proteins-between cells as a form of intercellular communication. Different EV types, including microvesicles (MVs), exosomes, oncosomes, and apoptotic bodies, have been characterized on the basis of their biogenesis or release pathways. Microvesicles bud directly from the plasma membrane, are 100 nanometers (nm) to 1 micrometer (μm) in size, and contain cytoplasmic cargo (Zaborowski, M P et al. BioScience (2015) 65 (8): 783-97, citing Heijnen, H F et al. Blood (1999) 94: 3791-99). Another EV subtype, exosomes, is formed by the fusion between multivesicular bodies and the plasma membrane, by which multivesicular bodies release smaller vesicles (exosomes) whose diameters range from 40 to 160 nm (Id., citing El Andaloussi, S. et al. Nature Reviews Drug Discovery (2013) 12: 347-57; Cocucci, E. and Meldolesi J. Trends in Cell Biology (2015) 25: 364-72). Dying cells release vesicular apoptotic bodies (50 nm-2 m) that can be more abundant than exosomes or MVs under specific conditions and can vary in content between biofluids (Id., citing Thery, C. et al. J. Immunology (2001) 1666: 7309-18; El Andaloussi, S. et al. Nature Reviews Drug Discovery (2013) 12: 347-57). Membrane protrusions can also give rise to large EVs, termed oncosomes (1-10 m), which are produced primarily by malignant cells in contrast to their nontransformed counterparts (Id., citing Di Vizio, D. et al. Am. J. Pathol. (2012) 181: 1573-84; Morello, M. et al. Cell Cycle (2013) 12: 3526-36).

The term “Fab fragment” as used herein refers to an antibody fragment composed of a single antigen-binding arm of an antibody without the Fc region, produced by cleavage of IgG by the enzyme papain. It contains the complete light chain plus the amino-terminal variable region and first constant region of the heavy chain, held together by an interchain disulfide bond.

The term “F(ab′)₂ fragment as used herein refers to an antibody fragment composed of two linked antigen-binding arms (Fab fragments) without the Fc regions, produced by cleavage of IgG with pepsin.

The term “fragment” or “peptide fragment” as used herein refers to a small part derived, cut off, or broken from a larger peptide, polypeptide or protein, which retains the desired biological activity of the larger peptide, polypeptide or protein. Antibody binding fragments (e.g., Fab, Fab′, F(ab′)₂, Fv, and single-chain (sc) antibodies) can be produced by recombinant DNA techniques, or by enzymatic or chemical cleavage of intact antibodies.

The term “forced vital capacity” or “FVC” is an objective measurement of respiratory muscle function. It refers to the maximal volume of gas that can be exhaled from full inhalation by exhaling as forcefully and rapidly as possible.

The term “healthy subject” as used herein refers to a subject having no signs or symptoms of a lung disease.

The term “gene” as used herein refers to a locatable segment of a genomic sequence corresponding to a unit of inheritance, which is associated with regulatory regions, transcribed regions that code for a protein or RNA product, and other functional sequence regions.

The terms “gene expression” and “expression” are used interchangeably herein to refer to the process by which inheritable information from a gene, such as a DNA sequence, is made into a functional gene product, such as protein or RNA.

The term “genetic engineering” as used herein refers to the use of molecular biology methods to manipulate nucleic acid sequences and introduce nucleic acid molecules into host organisms. The term “genetically engineered” as used herein means a cell that has been subjected to recombinant DNA manipulations, such as the introduction of exogenous nucleic acid molecule, resulting in a cell that is in a form not found originally in nature.

The term “heterogeneous” as used herein refers to being composed of unrelated or unlike elements or parts; varied; miscellaneous; of different kinds; differing or opposite in structure, quality etc; dissimilar.

The term “homogeneous” as used herein refers to being of the same character, structure, quality; etc.; essentially like; of the same nature; composed of similar or identical elements or parts; uniform.

The term “inhalation” as used herein refers to the process by which gases or air enter the lungs.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “join” as used herein means to link, couple, or connect one thing with another. Each of these terms is used interchangeably with the others.

The term “labeling” as used herein refers to a process of distinguishing a compound, structure, protein, peptide, antibody, cell or cell component by introducing a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

The term “long noncoding RNA” (“lncRNAs”) as used herein refers to a class of transcribed RNA molecules that are longer than 200 nucleotides and yet do not encode proteins. LncRNAs can fold into complex structures and interact with proteins, DNA and other RNAs, modulating the activity, DNA targets or partners of multiprotein complexes. Crosstalk of lncRNAs with miRNAs creates an intricate network that exerts post-transcriptional regulation of gene expression. For example, lncRNAs can harbor miRNA binding sites and act as molecular decoys or sponges that sequester miRNAs away from other transcripts. Competition between lncRNAs and miRNAs for binding to target mRNAs has been reported and leads to de-repression of gene expression (Zampetaki, A. et al. Front. Physiol. (2018) doi.org/10.3389/fphys.2018.01201, citing Yoon, J H et al. Semin. Cell Dev. Bio. (2014) 34: 9-14; Ballantyne, M D et al. Clin. Pharmacol. Ther. (2016) 99: 494-501). Finally, lncRNAs may contain embedded miRNA sequences and serve as a source of miRNAs (Id., citing Piccoli, M T et al. Cir. Res. (2017) 121: 575-83).

The term “lung” as used herein refers to one of a pair of organs occupying the pulmonary cavities of the thorax, and are the organs of respiration in which aeration of the blood takes place. Each lung is irregularly conical in shape, presenting a blunt upper extremity (the apex), a concave base following the curve of the diaphragm, an outer convex surface (costal surface), an inner or mediastinal surface, a thin and sharp anterior border, and a thick and rounded posterior border.

The term “lung function” is used herein to refer to a measure of how well the lung is working. There are several types of lung function tests, including spirometry, pulse oximetry, exercise stress test or arterial blood gas test. Additionally, hydroxyproline levels, lung density and total cell count in bronchoalveolar lavage fluid (BAL) may be used to assess lung function. It is to be understood that any one of these tests may be used in combination with another.

For example, lung function may be assessed by determining the amounts of polymorphonuclear leukocytes, neutrophil products, eosinophils, eosinophil products, activated alveolar macrophages, alveolar macrophage products, cytokines, chemokines, growth factors for fibroblasts, and immune complexes in BAL fluid in an untreated control, or relative to a patient at a time point prior to treatment, where a decrease in the amounts of polymorphonuclear leukocytes, neutrophil products, eosinophils, eosinophil products, activated alveolar macrophages, alveolar macrophage products, cytokines, chemokines, growth factors for fibroblasts, and/or immune complexes is indicative of an increase in lung function.

Hydroxyproline is a major component of collagen, where it serves to stabilize the helical structure. Because hydroxyproline is largely restricted to collagen, the measurement of hydroxyproline levels can be used as an indicator of collagen content. A decrease in hydroxyproline levels relative to an untreated control subject are indicative of an increase in lung function.

As used herein, the terms “marker” or “cell surface marker” are used interchangeably to refer to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell population.

The term “messenger RNA” (“mRNA”) as used herein refers to a coding RNA, which functions in protein translation.

The term “microRNA” (or “miRNA”) as used herein refers to a class of small, 18- to 28-nucleotide-long, noncoding RNA molecules. Their major role is in the posttranscriptional regulation of protein expression

The terms “next generation sequencing”, “NGS”, “massively parallel sequencing”, or “deep sequencing” as used herein describe a high-throughput method used to determine the nucleotide sequence of an individual's whole genome at once in an automated process. First a DNA library is prepared from a patient's sample by fragmentation, purification and amplification of the DNA sample. Individual fragments are then physically isolated by attachment to solid surfaces or small beads. The sequence of each of these fragments is resolved simultaneously by such techniques as sequencing by synthesis. The resulting sequence data are computationally aligned against a ‘normal reference’ genome.

The term “non-coding RNA” (“ncRNA”) as used herein refers to a functional RNA molecule that is transcribed from DNA but not translated into proteins. They are classified into housekeeping and regulatory noncoding RNAs. Housekeeping ncRNAs include ribosomal RNA (rRNA, the RNA component of ribosomes), transfer RNA (tRNA, which functions as an adapter for matching amino acids to mRNA), small nuclear RNA (snRNA, which functions in RNA processing such as mRNA splicing), and small nucleolar RNAs (snoRNAs, which functions in guiding chemical modification of other RNAs). Regulatory noncoding RNAs are divided into short ncRNAs (<200 nt) and long ncRNAs (>200 nts). Short noncoding RNAs <200 nt include microRNA (miRNA), small interfering RNAs (siRNAs) and piwi-associated RNAs (piRNAs), and long noncoding RNAs (>200 nt). [Losko, M. et al. Mediators of Inflammation (2016) 1-12. 10.1155/2016/5365209].

The term “non-invasive” as used herein refers to a medical procedure that does not require insertion of an instrument or device through the skin or a body orifice for diagnosis or treatment.

The term “nucleic acid” as used herein refers to a deoxyribonucleotide or ribonucleotide polymer in either single- or double-stranded form, and, unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” as used herein refers to a molecule consisting of a nitrogen-containing base (adenine, guanine, thymine, or cytosine in DNA; adenine, guanine, uracil, or cytosine in RNA), a phosphate group, and a sugar (deoxyribose in DNA; ribose in RNA).

The term “overexpressed” as used herein refers to increased quantity of a gene or gene product relative to a quantity of the gene or gene product under normal conditions.

The term “particle” as used herein refers to an extremely small constituent, e.g., microparticles (particles in the micrometer size range), nanoparticles (particles in the nanometer size range), etc.

The term “plasma” as used herein refers to the fluid (noncellular) portion of circulating blood, and the fluid portion of lymph.

The term “pleural effusion” as used herein refers to a buildup of fluid between the layers of tissue that line the lungs and chest cavity that can pevent the lungs frum fully inflating, making it hard to breathe.

The term “pleural fluid” refers to a liquid located between the layers of the pleura, a two-layer membrane that covers the lungs and lines the chest cavity. Pleural fluid keeps the pleura moist and reduces frictaon between the membranes when breathing. The area that contains pleural fluid is known as the pleural space. In healthy subjects, there is small amount of pleural fluid in the pleural space.

The terms “polypeptide” and “protein” are used herein in their broadest sense to refer to a sequence of subunit amino acids, amino acid analogs, or peptidomimetics. The subunits are linked by peptide bonds, except where noted. These terms also apply to amino acid polymers in which one or more amino acid residue is an artificial chemical analogue of a corresponding naturally occurring amino acid, as well as to naturally occurring amino acid polymers. The terms also are inclusive of modifications including, but not limited to, glycosylation, lipid attachment, sulfation, gamma-carboxylation of glutamic acid residues, hydroxylation and ADP-ribosylation. It will be appreciated, as is well known, that polypeptides may not be entirely linear. For instance, polypeptides may be branched as a result of ubiquitination, or they may be circular, with or without branching, generally as a result of posttranslational events, whether by natural processing or by events brought about by human manipulation, which do not occur naturally. Circular, branched and branched circular polypeptides may be synthesized by entirely synthetic methods.

The term “purification” and its various grammatical forms as used herein refers to a process of isolating or freeing from foreign, extraneous, or objectionable elements. The composition is nonetheless substantially pure in that it has been substantially separated from the substances with which it may be associated in living systems or during synthesis. As used herein, the term “substantially pure” refers purity of at least 75%, at least 80%, at least 85%, at least 90%, at least 95% or at least 99% pure as determined by an analytical protocol. Such protocols may include, for example, without limitation, flow cytometry, electrophoresis, small-RNA sequencing, quantitative PCR, nanoparticle tracking, electron microscopy, mass spectrometry, Western blotting, ELISA, and various metabolic assays.

The term “quantitative PCR” (or “qPCR”), also called “real time-PCR” or “quantitative real-time PCR” refers to a polymerase chain reaction-based technique that couples amplification of a target DNA sequence with quantification of the concentration of that DNA species in the reaction.

A recombinant or “engineered” nucleic acid molecule is a nucleic acid molecule that has been altered through human manipulation. As non-limiting examples, a recombinant nucleic acid molecule: 1) includes conjoined nucleotide sequences that are not conjoined in nature, 2) has been engineered using molecular cloning techniques such that it lacks one or more nucleotides with respect to the naturally occurring nucleic acid molecule sequence, or 3) has been manipulated using molecular cloning techniques such that it has one or more sequence changes or rearrangements with respect to the naturally occurring nucleic acid sequence.

A recombinant” cell or vector is one that has been modified by the introduction of a heterologous nucleic acid or a cell that is derived from a cell so modified. Recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation transduction/transposition) such as those occurring without deliberate human intervention.

The term “recombinant expression cassette” refers to a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, virus, or nucleic acid fragment. Typically, the recombinant expression cassette portion of an expression vector includes, among other sequences, a nucleic acid to be transcribed, a promoter, and a transcription termination signal such as a poly-A signal.

The term “recombinant host” refers to any prokaryotic or eukaryotic cell that contains either a cloning vector or an expression vector. This term also includes those prokaryotic or eukaryotic cells that have been genetically engineered to contain the cloned genes, or gene of interest, in the chromosome or genome of the host cell.

When applied to organisms, the term recombinant, engineered, or genetically engineered refers to organisms that have been manipulated by introduction of a heterologous or recombinant nucleic acid sequence into the organism, and includes gene knockouts, targeted mutations and gene replacement, promoter replacement, deletion, or insertion, as well as introduction of transgenes into the organism. The heterologous or recombinant nucleic acid molecule can be integrated into the recombinant/genetically engineered organism's genome or in other instances are not integrated into the recombinant/genetically engineered organism's genome.

The term “recombinant protein” as used herein refers to a protein produced by genetic engineering.

The term “serum” as used herein refers to the fluid portion of the blood obtained after removal of the fibrin clot and blood cells.

The term “SFTPC gene” refers to the gene that provides instructions for making surfactant protein C (SP-C), one of four proteins (each produced from a different gene) in surfactant, a mixture of phospholipids and proteins that lines lung tissue and makes breathing easier. Without normal surfactant, the tissue surrounding the alveoli (air sacs in the lungs) sticks together after exhalation (because of surface tension), causing the alveoli to collapse. As a result, filling the lungs with air on each breath becomes very difficult, and the delivery of oxygen to the body is impaired. Surfactant lowers surface tension, easing breathing and avoiding lung collapse. The SP-C protein helps spread the surfactant across the surface of the lung tissue, aiding in the surface tension-lowering property of surfactant.

The term “targeted” with regard to capture of EVs as used herein refers to the specific and selective isolation and purification of EVs by the EV-CATCHER technology.

The term “terminal respiratory unit” as used herein refers to all of the alveolar ducts, together with their accompanying alveoli that stem from the most proximal (first) respiratory bronchiole and contains approximately 100 alveolar ducts and 2000 alveoli. Because gse phase diffusin is so rapid, the partial pressures of oxygen and carbon dioxide are uniform throughout the unit.

The term “tetraspanin” as used herein refers to membrane-spanning proteins with a conserved structure that function primarily as membrane protein organizers. Members of the tetraspanin family of proteins have four transmembrane domains, which contribute to the creation of a small (EC1) and large (EC2) extracellular loop [Termini, CM, Gillette, J M, Front. Cell Dev. Biol. (2017) 5: 34, citing Abe, M. et al. Cancer Lett. (2008) 266: 163-70). The large extracellular loop contains a conserved Cys-Cys-Gly amino acid motif (CCG-motif), as well as two other conserved cysteine residues. Many members of the tetraspanin family also contain post-translational modifications.

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of the present disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without unacceptable side effects. Treatment also includes prolonging survival as compared to expected survival if not receiving treatment.

The term “tidal volume” as used herein refers to the amount of air that moves in or out of the lungs during a normal breath. It measures about 500 mL in an average healthy adult human male, and approximately 400 mL in an average healthy adult human female.

The term “underexpressed” as used herein refers to decreased quantity of a gene or gene product relative to the quantity of a gene or gene product under normal/non-disease conditions.

A “variant” of a peptide or protein is a peptide or protein sequence that varies at one or more amino acid positions with respect to the reference peptide or protein. A variant can be a naturally-occurring variant or can be the result of spontaneous, induced, or genetically engineered mutation(s) to the nucleic acid molecule encoding the variant peptide or protein. A variant peptide can also be a chemically synthesized variant.

### Embodiments

According to one aspect, the present disclosure provides a non-invasive method for early detection of deep lung pathology in a subject at risk of the deep lung pathology. According to some embodiments, the method comprises
(a) sampling deep lung tissue of the subject at risk of the deep lung pathology by isolating micro RNA (riRNA) cargo of lung tissue origin in exhaled extracellular vesicles (EVs) purified from exhaled breath condensates (EBCs) of the subject;
(b) purifying lung-specific exhaled extracellular vesicles in exhaled breath condensate by antibody capture of the lung-specific exhaled EVs; and
(c) detecting the pathology by comparing an miRNA profile of the exhaled extracellular vesicles of lung tissue origin purified from the subject to the profile of exhaled extracellular vesicles purified from exhaled breath condensates of a healthy subject. According to some embodiments, the early detection can lead to early treatment of the pathology and an improved health outcome for the subject.

According to some embodiments, the miRNA comprises tissue-specific surface proteins derived from terminal bronchioles and alveoli. According to some embodiments, the proteins detected on a surface of exhaled EVs comprise club cell secretory protein (CCSP), type 2 pneumocyte marker surfactant protein C (SFTPC) or both.

According to some embodiments, the EVs are purified from exhaled breath condensate by antibody capture of the lung specific exhaled EVs in the EBC.

According to some embodiments, the method for purifying lung specific exhaled extracellular vesicles in exhaled breach condensate by antibody capture of the lung-specific exhaled EVs comprises
(i) activating the antibody with a dibenzocyclo-octyl (DBCO)-ester to form a DBCO-modified antibody;
(ii) coupling the DBCO-modified antibody to a DNA linker by click chemistry,
(iii) binding the antibody-DNA linker conjugates to streptavidin coated well plates pretreated with RNAse A;
(iv) releasing the purified populations of deep lung-specific EVs from the streptavidin-coated well plates enzymatically by uracil glycosylase; and
(v) eluting the purified population of EVs from the antibody complex by contacting the complex with free tissue specific antigen.

According to some embodiments, the antibody is a monoclonal antibody raised against CCSP [Uteroglobin/SCGB1A1 (CCSP) Antibody—Novus Biologicals—Catalog #MAB4218]. According to some embodiments, the antibody is a monoclonal antibody raised against SFTPC [Prosurfactant Protein C (SFTPC) Antibody—Novus Biologicals—Catalog #NBP1-87201]. According to some embodiments, both a monoclonal antibody raised against CCSP and a monoclonal antibody raised against SFTPC are used to purify the EVs from EBCs. According to some embodiments, the purification steps for each antibody proceed sequentially.

According to some embodiments, profiles of the miRNA cargo isolated from the exhaled breath condensates by antibody capture correlate with profiles of miRNA purified from bronchoalveolar lavage fluid obtained from the subject.

According to some embodiments, the deep lung pathology cojprises a lung dysfunction due to smoking.

According to some embodiments, the deep lung pathology comprises a lung dysfunction due to asthma.

According to some embodiments, the deep lung pathology comprises a lung dysfunction due to a lung cancer.

According to some embodiments, examples of potential health outcomes from early detection include, without limitation, a reduction in deep lung-related related mortality and an increased life expectancy

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described.

It must be noted that as used herein and in the appended claims, the singular forms “a”, “and”, and “the” include plural references unless the context clearly dictates otherwise.

All publications mentioned herein are incorporated herein by reference to disclose and described the methods and/or materials in connection with which the publications are cited.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Clinical Specimen Collection

### Subject Recruitment and Specimen Collection at the Montefiore Medical Center (MMC):

A longstanding RTube™-based collection procedure was used at the Montefiore-Einstein Medical Center/Comprehensive Cancer Center for the collection of exhaled breath condensate (EBC), mouth rinse, buccal brushings, bronchial brushings and bronchoalveolar lavage (BAL) from an initial 18 subjects undergoing clinically indicated bronchoscopy, under IRB protocol (#2007-407). In person interviews were conducted, clinical data obtained and verified, and noninvasive EBC, mouth rinse and buccal brush specimens, were collected before the clinically indicated bronchoscopic procedure. All EBC collections were performed using RTube™ breath condensate collection devices (Respiratory Research Inc, Cat #2501). This single-use FDA-registered device is comprised of a coated plastic collection tube and a mouthpiece where during inhalation air enters the base of the mouthpiece where air can be inhaled (the air enters the mouthpiece through a filter at its base) and during exhalation the breath is directed upward into a polypropylene tube where exhaled droplets can be condensed. The collection tube contains is a unique custom duckbill valve, which opens only during exhalation and seals during inhalation, thereby ensuring one-way condensation of EBC into the collection tube. Moreover, as the duckbill is in contact with the collection tube via a plastic O-ring, which can be plunged upward into the collection tube to pool the condensed EBC, thereby avoiding cumbersome centrifugation of the tube in order to collect its' EBC content. It is also important to note that the design of the large “T” section of the mouthpiece ensures that saliva is separated from exhaled breath and does not enter the collection tube. During EBC collection, a chilled aluminum cylinder covered with a cloth sleeve is positioned over the collection tube. The chilled cylinder allows for the exhaled droplets to condense and deposit on the inner surface of the collection tube. We have observed that over the course of 10 minutes, a healthy subject breathing without excessive exertion or hyperventilation will generate an average of 1.5 to 2 ml of EBC biofluid. Mouth rinse was collected by 5 ml standard commercial EtOH-containing mouth rinse and buccal brushings were obtained by means of inner cheek swabs using sterile cytologic brushes. During the bronchoscopy procedure, research-devoted bronchoalveolar lavage was obtained by means of normal saline (NS) lavage of 40 ml input with ˜10-20 ml return and endobronchial brushings were collected using sterile cytologic brushes by means of standard clinical procedures. Following the analyses on the first 18 subjects we expanded our cohort analyses to include matched EBC and BAL that were collected from an additional 51 subjects (i.e., a total of 69 EBC and BAL specimens were analyzed in this study) in the same manner as described above. Specimens were snap frozen on dry ice immediately upon collection in the pre-procedure and bronchoscopy suite. Longer term storage was maintained at −80° C.

### Subject Recruitment and Specimen Collection at Hackensack University Medical Center (HUMC):

a. EBC samples were collected from (i) six treatment naïve stage IV lung cancer patients receiving treatment at the John Theurer Cancer Center (JTCC) at Hackensack University Medical Center (HUMC) and (ii) twelve healthy volunteers under an IRB-approved protocol (Pro #2020-0258). EBC collection from lung cancer patients (n=6) and healthy volunteers (n=12) was performed using the RTube™ breath condensate collection device and the above-described procedure at a single 10-minute period unless otherwise stated. EBC specimens were all processed within 1 hour of collection and stored as 500 μl aliquots at −80° C.

### Spectradyne Microfluidic Resistive Pulse Sensing (MRPS)

The particle size distribution of exh-EVs isolated from EBC was measured using MRPS on the Spectradyne nCS1 instrument (Spectradyne LLC, Signal Hill CA). The microfluidic system was initially primed with a solution of 0.2 μm filtered DPBS containing 1% Tween 20 (v/v). 2 μl of purified exh-EVs were loaded into TS-400 cartridges, which allow for the analysis of particles between 65 and 400 nm and instrument pressure and voltage parameters were determined automatically by the instrument software. Following each acquisition of data from >10,000 particle detection events for each sample, data were combined into a single stats file, and using the nCS1 Data Viewer software and peak filters and background subtraction were applied, according to manufacturer recommendations. Peak filters set were (i) transit time <60 s, (ii) diameter >65 nm, and signal to noise ratio (S/N) >10 for all samples. Additionally, combined stats files were analyzed for size distribution and particle concentration and peak-filtered CSD graphs were generated.

### Ultracentrifugation of EBC for Concentration and Isolation of Exh-EVs

a. In order to initially characterize exh-EVs contained within EBC, we collected 60 ml of EBC from each of two healthy donors, using the RTube™ breath condensate collection devices, with three 20-min collection periods per day for 5 consecutive days from each donor. After each collection period EBC was collected into individual Eppendorf tubes and stored at −80° C. until the total EBC volume of 60 ml was achieved from each donor. Once collected the 60 ml of EBC obtained from each donor was defrosted and combined separately prior to downstream concentration and processing. In order to concentrate and purify all exh-EVs from the 60 ml EBC, the gold standard sequential centrifugation approach for EV isolation was used [68]. Briefly, centrifugation was performed at 300×g at 4° C. for 5 min, followed by 10 min at 2,000×g at 4° C. to remove any potential larger cellular debris. The supernatant was then centrifuged at 10,000×g at 4° C. for 30 min to remove any macrovesicles and after being transferred to a clean ultracentrifuge tube, the supernatant was centrifuged at 100,000×g for 90 min at 4° C. The resulting supernatant was discarded, and the exh-EV pellet was resuspended in 10 mL of sterile 1×PBS and again centrifuged at 100,000×g for 90 min at 4° C. The final resulting exh-EV pellet from each donor split into three for downstream EV-CATCHER isolations and exh-EV characterization experiments.

### Western Blot Analysis

Western blot analyses were conducted to characterize the presence of common EV protein markers present on exh-EVs isolated from EBC. Isolated exh-EVs purified by means of ultracentrifugation, tTSP (CD9/CD63/CD81) EV-CATCHER and CCSP/SFTPC EV-CATCHER assays were separated on 4-12% polyacrylamide precast mini-PROTEAN TGX gels (Bio-Rad, #4561086) by sodium dodecyl sulphate polyacrylamide gel electrophoresis (SDS-PAGE). 5 μl of PageRuler™ Plus prestained Protein ladder (ThermoFisher, #26620) was loaded and used for gel orientation and determination of molecular weights of separated proteins. 10 μg of each purified recombinant protein was loaded, and gels were run at 100 V for 90 min (Power Pac 300, Bio-Rad) in 1× Tris/Glycine/SDS buffer (Bio-Rad, #1610732). After proteins were separated, gels were UV activated on a Chemi-Doc™ MP (Bio-Rad) system to allow for the activation UV-dependent 2,2,2,-trichloroethanol (TCE) labelling of tryptophan residues in proteins and thus their stain-free visualization. Proteins were then transferred to 0.2 μm polyvinylidene fluoride (PVDF) membranes (Bio-Rad, #1704156) using a semi-dry electro-transfer system (TransBlot Turbo v1.02, Bio-Rad) for 30 min at 25 V. Proteins were visualized using the stain-free blot protocol provided on a Chemi-Doc™ MP (Bio-Rad) system to evaluate protein transfer and membranes were blocked using EveryBlot blocking buffer (Bio-Rad, #12010020) for 30 min. Membranes were incubated at 4° C. O/N with TBS-T (1×TBS, pH 6.8, 0.1% Tween20) diluted anti-mouse primary antibodies targeted against Apolipoprotein B (Novus Biologicals, Cat #MAB4124), Albumin (Novus Biologicals, Cat #MAB1455), CD63 (Abcam, Cat #ab59479), or with anti-rabbit primary antibodies targeted against Apolipoprotein A1 (Novus Biologicals, Cat #MAB36641), CD81 (Abcam, #ab233692), and CD9 (Abcam, #ab263023). Membranes were washed with TBS-T (3×5 min) before incubation in anti-mouse IgG horseradish peroxidase conjugated secondary antibodies for 1 h, with gentle agitation at RT. Membranes were washed with TBS-T (3×5 min) before proteins were detected using SuperSignal™ West Femto Maximum Sensitivity Substrate (Pierce, #34095) and protein bands were visualized using ImageLab 4.0 software on a Chemi-Doc MP (Bio-Rad) imaging system.

### EV-CATCHER Isolation of the Exhaled Extracellular Vesicles

The isolation of exhaled EVs was performed using the EV-CATCHER isolation protocol described by Mitchell et al., as also described in U.S. patent application Ser. No. 17/560,909 (published as US2022-0205990) and WO2022/140662, using a combination of CD9/CD63/CD81 (i.e., triple tetraspanin (tTSP) exh-EV isolation) or CCSP/SFTPC (i.e., lung specific exh-EV isolation) as the capture antibodies [68]. Briefly, equimolar amounts of 5′-Azide modified and 3′-Biotin modified oligonucleotides (Integrated DNA Technologies) were annealed in 1×RNA annealing buffer (60 mM KCl, 6 mM HEPES pH 7.5, 0.2 mM MgCl2), prior to separation on a 15% non-denaturing polyacrylamide (PAGE) gel. The annealed double stranded (ds) DNA product was visualized on a blue light box with SYBR® Gold™ dye (ThermoFisher, #S11494), excised, crushed using a gel breaker tube (IST Engineering, #3388-100), resuspended in 400 mM NaCl and placed on a thermomixer set to 4° C. and 1,100 RPM overnight (O/N). The solution was filtered, and the dsDNA linker was purified using the QIAEX® II gel extraction kit (Qiagen, #20021) according to manufacturer instructions. Capture antibodies (1 mg/ml) used for exh-EV pulls, were activated using 5 μl of freshly prepared 4 mM DBCO-NHS ester (Lumiprobe, #94720) and incubated for 30 min at room temperature (RT) in the dark. Reactions were stopped by adding 2.5 μl of 1M Tris-Cl (pH 8.0) at RT for 5 min in the dark. DBCO-activated antibodies were then desalted using Zeba desalting columns (ThermoFisher, #89882). and quantified on a Nanodrop 2000 instrument prior to the preparation of antibody-dsDNA (Ab-dsDNA) stock solutions (i.e., 100 μg of activated antibody conjugated to 50 μg of purified DNA linker. The Ab-dsDNA conjugates were then bound to streptavidin coated 96-well plates (Pierce, #15120) by incubating 1 μg of each Ab-dsDNA in 100 μl PBS per well (2 wells were prepared per sample). Solutions were carefully removed, and wells were washed three times with cold 1×PBS solution, prior to addition of RNase-A (12.5 μg/ml) treated samples (100 μl). Plates were sealed using microAMP optical adhesive film (Applied Biosystems, #4311971) and placed on a shaker at 300 RPM at 4° C., O/N. Samples were carefully removed, wells were washed 3 times with cold 1×PBS and 100 μl of freshly prepared uracil glycosylase (UNG) enzyme (ThermoFisher, #EN0362) in 1×PBS (1×UNG buffer (200 mM Tris-Cl (pH 8.0), 10 mM EDTA and 100 mM NaCl), with 1 unit of enzyme) was added to each well. Plates were incubated at 37° C. for 2 h on a shaker at 300 RPM for UNG digestion of the dsDNA linker, and exh-EVs were collected for downstream analyses.

### Transmission Electron Microscopy

Transmission electron microscopy (TEM) of exhaled EVs purified by ultracentrifugation and the EV-CATCHER assay was performed at the analytical imaging facility at the Albert Einstein College of Medicine. Briefly, purified exh-EVs were fixed using 2% glutaraldehyde in phosphate buffer (Electron Microscopy Services, #6536-05) and stored at 4° C. 300 mesh formvar-coated grids were inverted onto 20 μl of fixed exh-EV suspensions for 2 min and wicked dry. Grids were then inverted onto 40 μl of 2% aqueous uranyl acetate for 1 min, and wicked dry. Samples were imaged on a JEOL JEM-1400+ transmission electron microscope (JEOL Ltd.; Tokyo, Japan) operating at an accelerating voltage of 80 kV. High resolution TIFF images were acquired and saved using an AMT 16 MP digital camera system (Advanced Microscopy Techniques Corp.; Woburn, MA).

### ONi Super Resolution Nanoimaging

Purified exh-EVs were processed for imaging on the ONi super resolution Nanoimager using the ONi EV Profiler kit v2.0 according to manufacturer's instructions. Briefly, the surface of the assay capture chip was prepared by applying 5 pl of S3 buffer to each lane and incubated at room temperature for 10 min. 30 pl of W1 was then applied to each lane to remove excess S3, after which 10 pl of S4 buffer was slowly pipetted to each lane ensuring that no bubbles were introduced into lanes. After a 10-min incubation period at room temperature lanes were again washed by applying 30 pl of W1 buffer to each lane. EV capture was then performed by immediately applying 10 pL of ultracentrifuge and tTSP (CD9/CD63/CD81) EV-CATCHER and CCSP/SFTPC EV-CATCHER purified exh-EVs and allowing binding to occur for 15 min. Lanes were then washed using 30 pl of W1 buffer and captured EVs were fixed by adding 20 pl of F1 to each lane and incubating the chip at room temperature for 10-minutes. The staining of captured exh-EVs was performed by firstly preparing a three-antibody working solution comprising the CD63-Alexa Fluor™ 568, CD9-Alexa Fluor™ 488 and CCSP-Alexa Fluor™ 647 antibodies, or the CD63-Fluor™ 568, CD9-Alexa Fluor™ 488 and SFTPC-Alexa Fluor™ 647 antibodies, combined together in W1 buffer so that each antibody is at a dilution of 1:20. The final staining solution was prepared by combining 1 pl of the prepared working solution with 9 pl of N1 buffer for each lane, gently pipetting to mix the solution and applying 10 pl to each lane of the EV profiler chip and allowed to incubate for 50 minutes at room temperature in the dark. Immediately following antibody incubation lanes were washed with 30 pl of W1 buffer followed by a 20-min incubation with 20 pl of F1 buffer for 10 minutes. A final wash step was performed and BCubed™ dSTORM imaging buffer added to each well immediately before EV profiler chips were imaged. Image acquisition on the ONi super resolution Nanoimager was performed in the NimOS Light program with a 640 dichroic split using the following parameters: 640 nm laser set to 20-30% laser power, the 560 nm laser at 35% laser power and the 473/488 nm laser set to 70% laser power. The number of runs (frames) for all laser lines was set to 1000 and all image analyses were performed using CODI software.

### RNA Extractions

Small-RNA from exhaled EVs was isolated using the miRNeasy Serum/Plasma kit (Qiagen, Cat #217184) according to manufacturer's instructions with some modifications to improve total RNA yield. Briefly, QIAzol was added to 100 pl of exh-EVs, vortexed and incubated at RT for 3 min, after which chloroform was added to each sample. Samples were vortexed again and incubated at RT for 3 min. Samples were centrifuged at 12,000×g, at 4° C. for 15-min and the upper aqueous phase of each sample was carefully removed and transferred into new siliconized tubes, to which 1.7× volume of 100% ethanol and 2 pl of small RNA size markers (19nt and 24nt 1.5 ng each) were added per sample. Samples were incubated on ice for 40 min prior to column purification and then passed twice through RNeasy minElute columns, followed by a working solution of RPE wash buffer, and then ice cold 80% ethanol. Columns were spun to remove residual ethanol and total RNA was eluted with 50 pl of RNase-free water. Samples were then speed-vacuumed to 10 μl prior to small-RNA sequencing.

Small-RNA cDNA Library Preparations

Small-RNA sequencing of RNA extracted from mouth-rinse, buccal brushings, exhaled breath condensates (EBC) and/or purified EBC exh-EVs, bronchial brushing, and bronchoalveolar lavage (BAL) and/or purified BAL EVs were performed using the cDNA library preparation protocol described by Loudig et al. (2017), with modifications for low input RNA from purified EVs [69]. In brief, total RNA from 18 samples, recovered from 100 LIII biofluids or exh-EVs purified from whole EBC or BAL biofluids underwent individual ligations using truncated K227Q T4 RNA Ligase 2 with 18 different adenylated barcoded 3′ adapters in separate 1.5 ml Eppendorf tubes, overnight at 4° C. The following day, the ligations were heat deactivated, combined, precipitated on ice with 100% ethanol, and centrifuged for 1 h at 14,000 rpm on ice at 4° C. The RNA was dried, resuspended, small-RNAs were separated on a 15% Urea-PAGE gel, excised, and incubated in a 400 mM NaCl solution at 4° C. on a thermomixer at 1,100 RPM. The next day the RNA was precipitated in presence of 100% ethanol, centrifuged, pelleted, resuspended, prior to being subjected to a second ligation with a 5′ adapter using T4 RNA Ligase 1 for 1 h at 37° C. The ligated product was separated on a 12% Urea-PAGE gel, ligated small-RNAs were excised, and incubated overnight in a 300 mM NaCl solution with 1LIIl of 100 μM 3′ PCR primer, which was also the reverse-transcription primer, on a thermomixer at 1,100 RPM at 4° C. The next day, the solution was filtered, precipitated with 100% ethanol, incubated on ice for 1 h, pelleted by centrifugation at 14,000 RPM for 1 h at 4° C., and a portion of this solution subjected to reverse transcription. A pilot PCR reaction was set up using the cDNA and the generation of amplicons was evaluated at cycles 10, 12, 14, 16, 18, 20 and 22, prior to analysis on a 2.5% agarose gel, and identification of the optimal PCR amplification cycle. A large-scale PCR reaction was set up using the cDNA for amplification, separation on a 2.5% agarose gel, isolation, and quantification on an Agilent Bioanalyzer. The small-RNA cDNA libraries were sequenced on an Illumina HiSeq-2500 sequencer, and the generated FASTQ files were processed for adapter trimming and small-RNA alignment to the hg-19 genome using the pipeline established in the Tuschl laboratory [82]. Read counts were normalized to total counts and subjected to statistical analyses (see below).

### Sequencing Data Analysis

The sequencing data analysis involved processing raw FASTQ files obtained from an Illumina HiSeq2500 sequencer. This processing was carried out using the RNAworld server, hosted at the Tuschl Laboratory, Rockefeller University. This processing encompassed tasks such as adapter trimming, read alignment, annotation and counting. Subsequently, miRNA analyses were executed using specialized Bioconductor packages within the R platform. To visualize the data, heat maps were generated from transformed counts utilizing the ‘NMF’ package, specifically the ‘aheatmap’ function. Differential expression was conducted employing the ‘DESeq2’ package. To filter out lowly-expressed miRNAs, the approach described by Chen et al was implemented within the ‘edgeR’ package. Notably, the differential expression models incorporated a batch variable (library) when necessary to address potential batch-related biases. To enhance the discriminative potential of miRNA profiles, a unique score, termed the ‘miRNA score’ was computed a distinct score for each sample, [83]. This score was derived by aggregating the standardized levels (z-values) of upregulated miRNAs and the negative z-values of downregulated miRNAs, either considering all miRNAs or only those shown to be differentially expressed at a specified significance cutoff in the specified condition.

### Results

### Exhaled Breath Condensates and Study Design

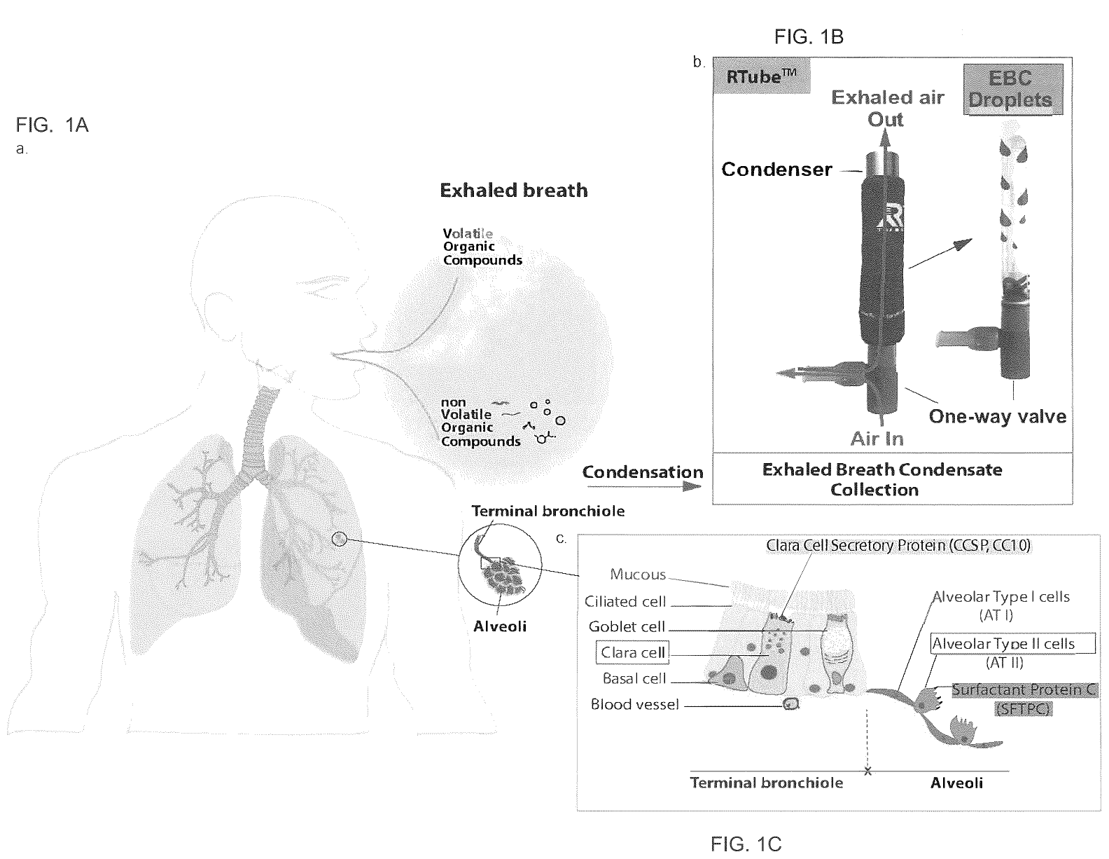

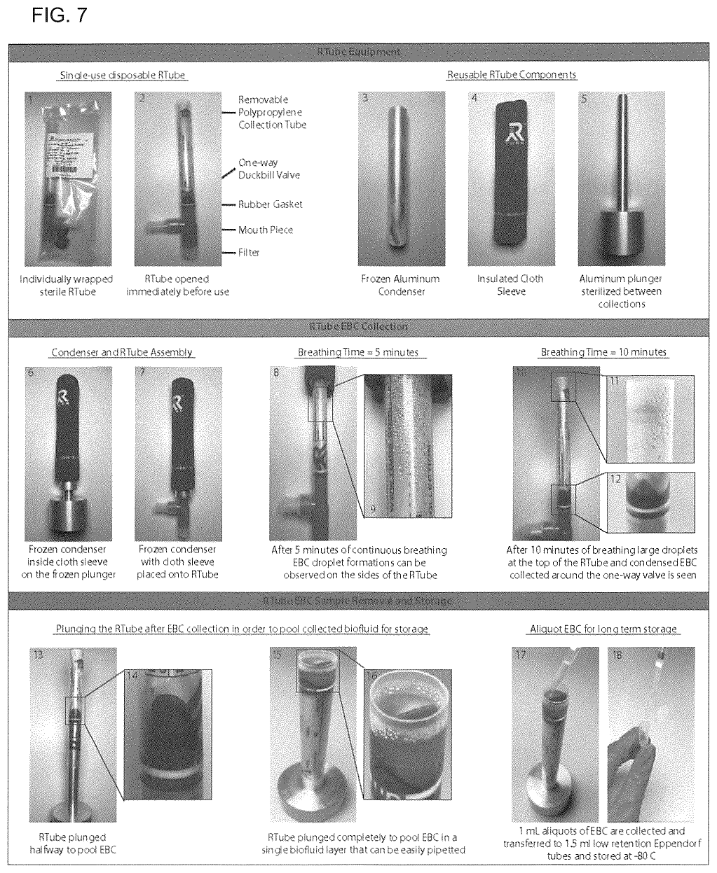

The RTube™ Exhaled Breath Condensate collection device, an FDA-registered single-use disposable handheld device (FDA #3004852415), was utilized for EBC collections from all subjects included in this study (FIG. 1B). The detailed process of EBC collection, using the RTube™ Device, is detailed in FIG. 7. Anatomically the terminal respiratory unit can be divided into the terminal bronchioles and the alveoli air sacs (FIG. 1C, left). Terminal bronchioles are comprised of different epithelial cell types, including the non-ciliated bronchiolar secretory Clara cells, which uniquely express Clara cell specific protein (CCSP) in the lung; and respiratory alveoli that are only comprised of two cell types; the alveolar type II cells that uniquely produce surfactant proteins (i.e., A1/A2, B, D) and particularly Surfactant Protein C (SFTPC), and alveolar type I cells that are involved in gas exchange between the blood and the external environment (FIG. 1C, right).

### Purification and Validation of Exhaled EVs (Exh-EVs) in EBC

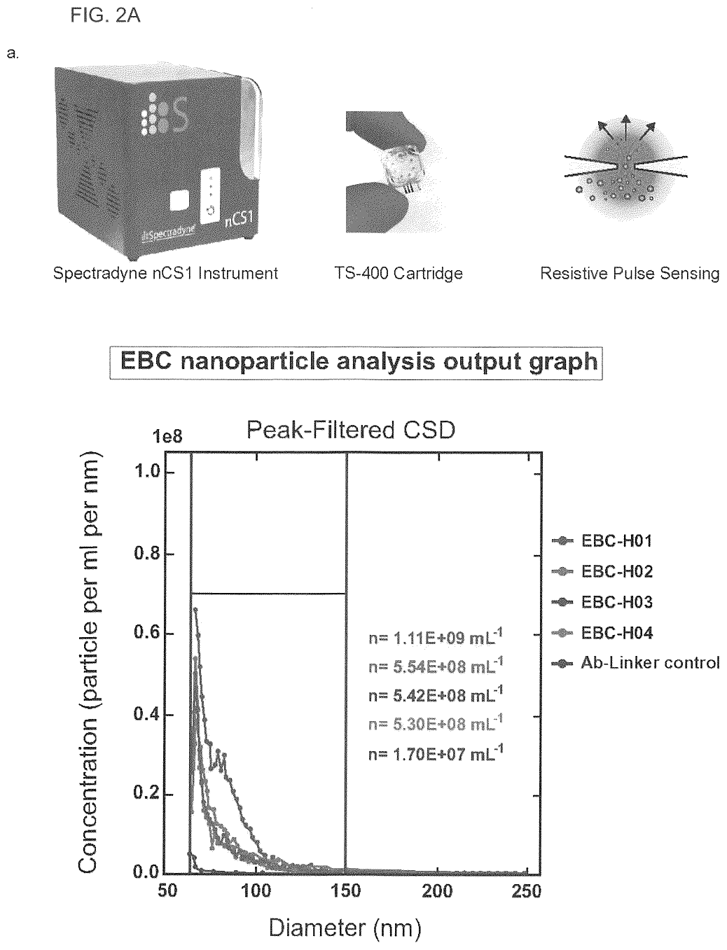

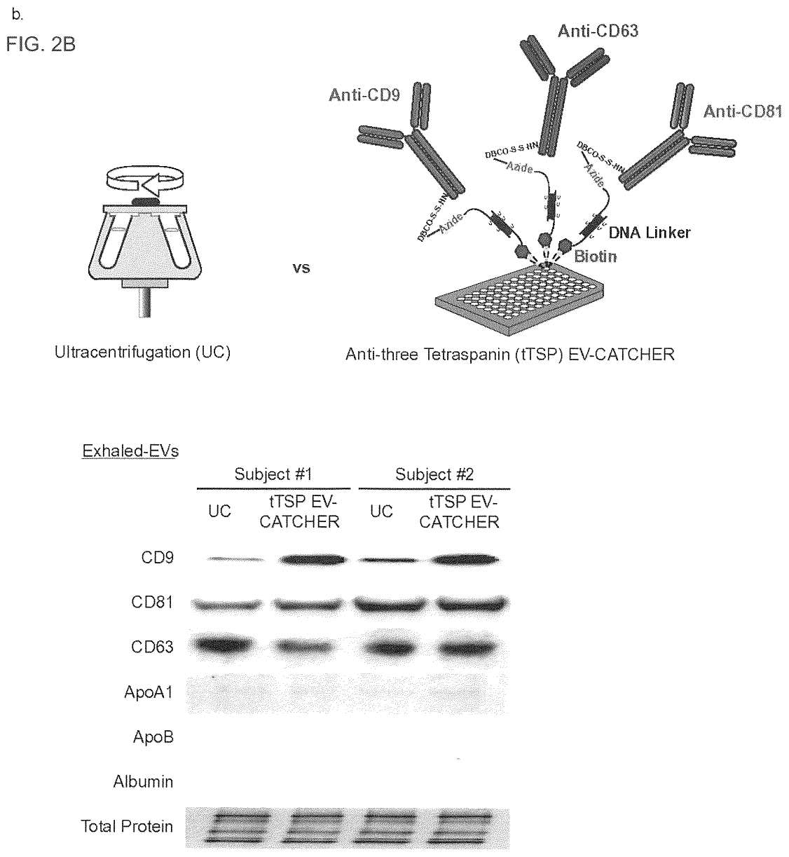

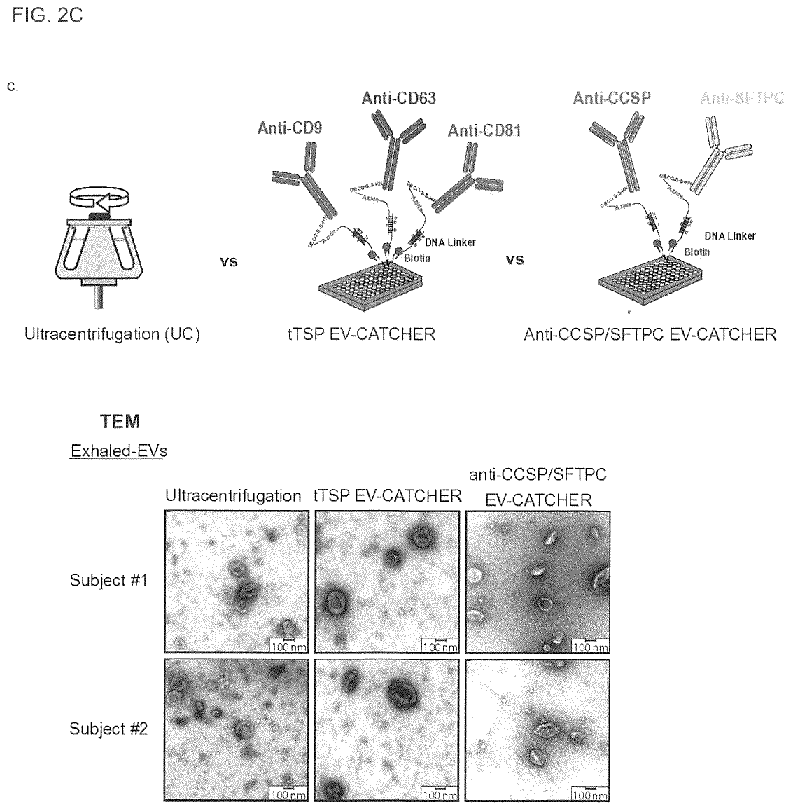

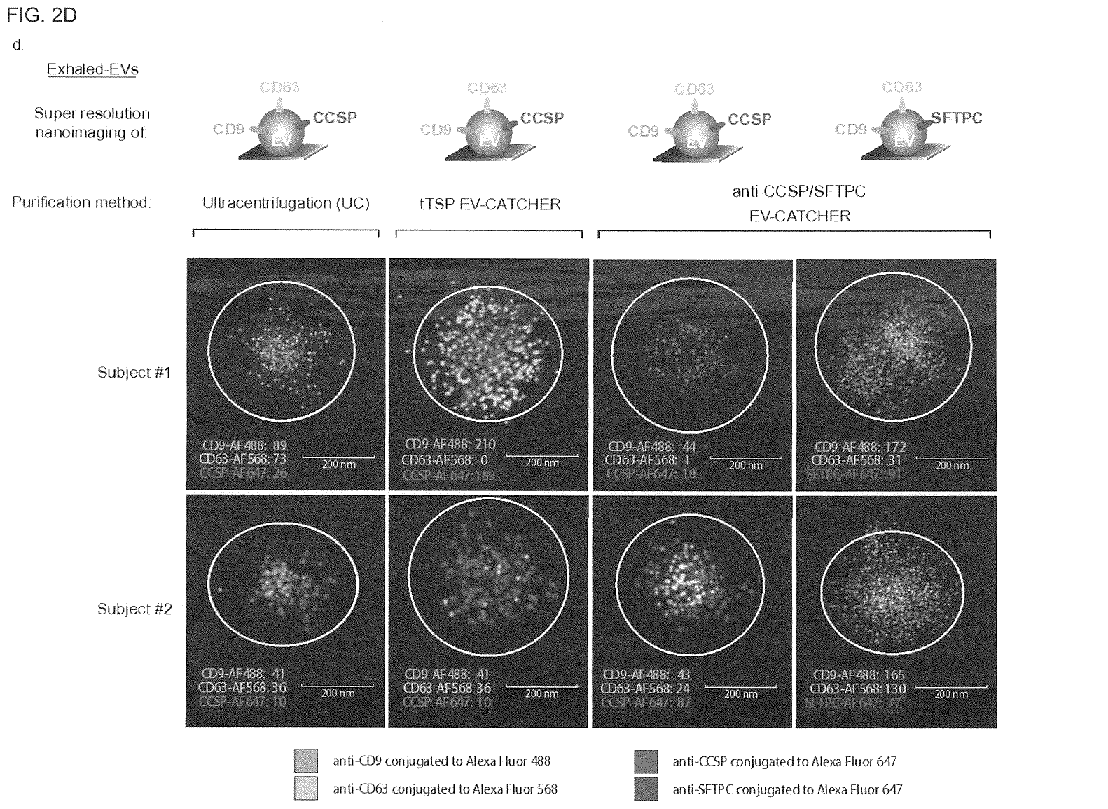

We first sought to determine whether EBC collected with the RTube™ device allowed for the condensation of exh-EVs. For this, we evaluated the purification of exh-EVs from the EBC collected from four healthy volunteer donors using our customized human anti-CD63 EV-CATCHER assay, which allows for the selective purification of EVs that harbor the CD63 tetraspanin from biofluids, as we previously described [80]. After selectively evaluating the capture of CD63 positive exh-EVs from the four EBC samples (FIG. 2A, green, red, light blue, and purple plots), we analyzed and counted the particle content of our immune-purified samples, and negative control (anti-CD63 antibody released without EVs after incubation with 1×PBS; FIG. 2A, dark blue) on a Spectradyne nCS1 instrument using TS-400 or C400 cartridges (which targets nanoparticles ranging between 65 nm and 400 nm). Our analyses revealed the presence of nanoparticles, which fell within the expected size range of exh-EVs (65-150 nm). Next, following MISEV2018 guidelines, we sought to characterize the identity of exh-EVs purified from EBC collected from two healthy volunteers (60 ml EBC progressively collected per subject) following gold-standard ultracentrifugation (UC) and antibody-based targeted purification using a combination of three tetraspanin antibodies (anti-CD9, -CD63, and -CD81) using our triple tetraspanin (tTSP) EV-CATCHER assay. We conducted Western blot analyses of exh-EVs purified by UC and by our tTSP EV-CATCHER assay, which validated the presence of common EV tetraspanin protein markers (i.e., FIG. 2B, see CD9, CD63, and CD81) with minimal to no visual detection of liposomal contaminating proteins, which include ApoA1 and ApoB, and no detection of albumin, a protein usually found as a high contaminant for EVs purified from blood but absent in our EBC samples (FIG. 2B). Next, we conducted Transmission Electron Microscopy (TEM) analyses of exh-EVs purified both by UC and our tTSP EV-CATCHER assay from the EBC of our two volunteer donors. Our TEM data confirmed both the presence and morphology of EVs (i.e., nanoparticles with a cup shaped membrane) in EBC, with sizes ranging between 65-150 nm (FIG. 2C, left and center panels), consistent with those described in the literature for EVs [84].

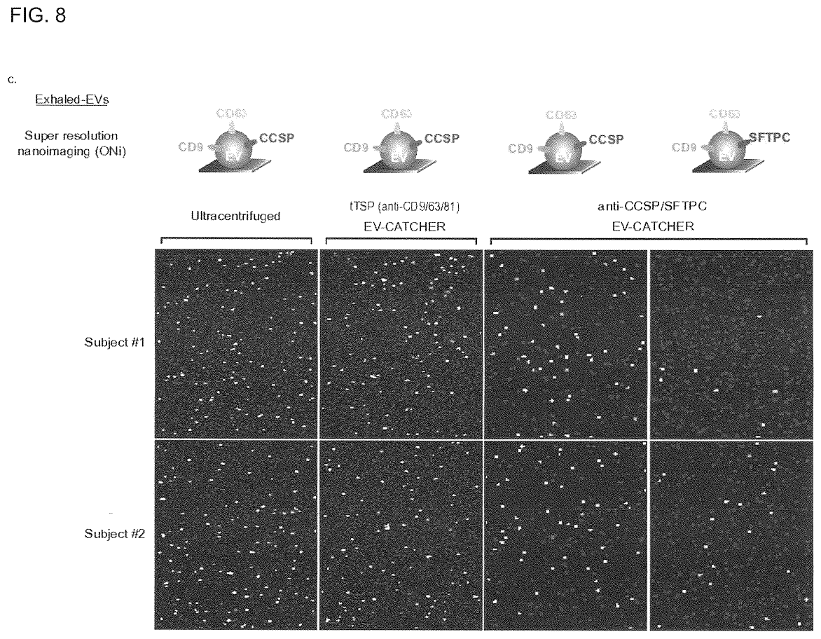

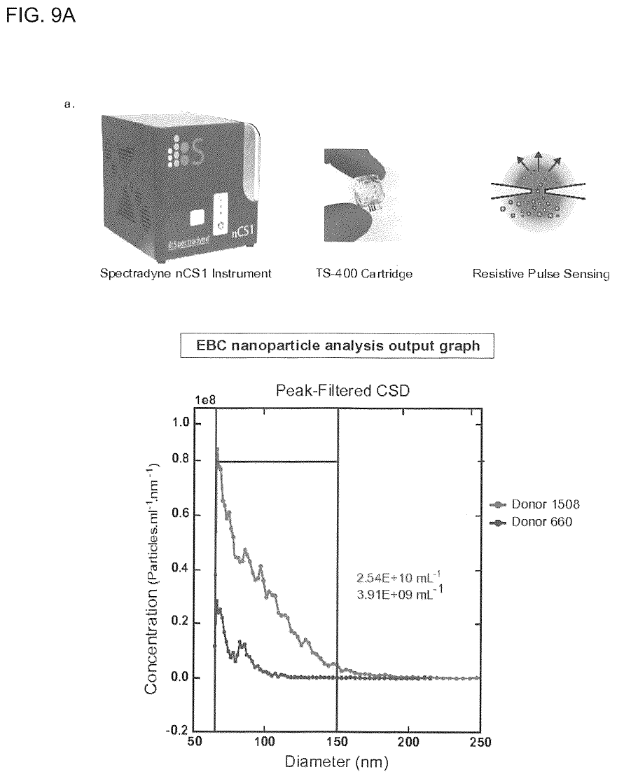

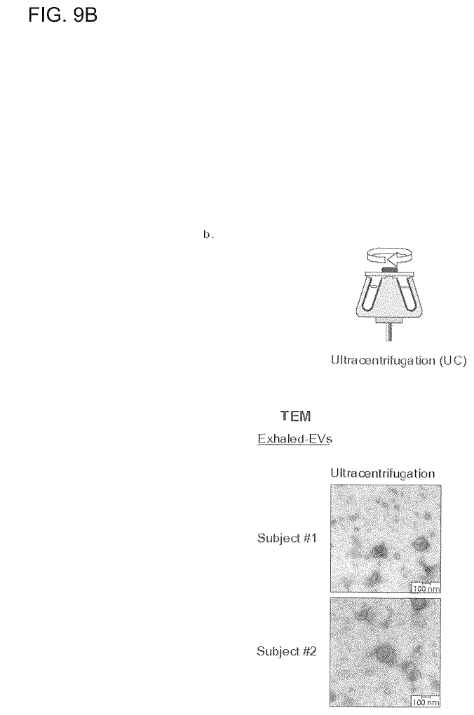

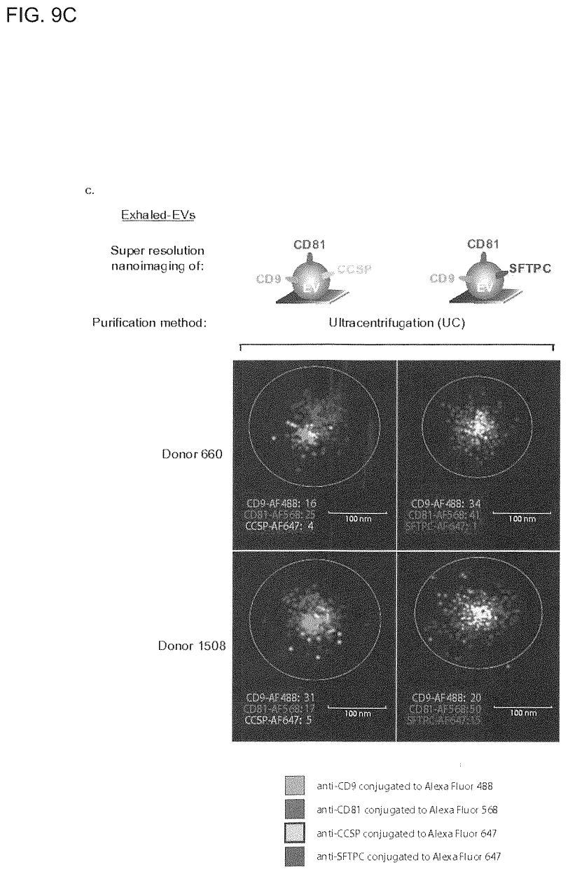

Because previous reports had indicated that EBC contains the terminal bronchiole Clara cell CCSP protein and the Alveolar type II cell-derived SFTPC [40], we sought to determine whether exh-EVs originating from the lung could be targeted using these two proteins as antigens and purified from EBC. Thus, we customized our EV-CATCHER assay with anti-CCSP and anti-SFTPC antibodies and purified EVs from ultracentrifuged pellets from our two EBC samples (i.e., healthy volunteers). Our TEM analyses validated that our anti-CCSP/anti-SFTPC customized EV-CATCHER assay allowed for the purification of exh-EVs from EBC (FIG. 2C right panels). Therefore, next we aimed to verify that these purified exh-EVs indeed harbored these secretory proteins on their membrane surface. To confirm the localization of two tetraspanins (CD9 and CD63; FIG. 2D left and center panels) and the two distinct secretory proteins (CCSP and SFTPC), we employed super-resolution nanoimaging (ONi) (FIG. 2D, right panels) to image these four proteins. We determined that CCSP, which is found in non-ciliated bronchiolar Clara cells, was located on the surface of exh-EVs purified by UC (FIG. 2D, left panels), our tTSP EV-CATCHER assay (FIG. 2D, center panels), and our anti-CCSP/SFTPC EV-CATCHER assays (FIG. 2D, left panels). Furthermore, we determined that SFTPC, which is produced by Alveolar Type II cells, could also be detected on the surface of exh-EVs (FIG. 2d, right panel). Evaluations of the complete fields of EV captures on the ONi revealed different distributions of triple positive CD9, CD63, CCSP and CD9, CD63, SFTPC, showing an apparent lower abundance of the latter within our two EBC samples (see FIG. 8). As another important validation (see FIG. 9 and to further confirm the origin of exh-EVs, we conducted nanoparticle analysis with a Spectradyne nCS1 instrument using the TS-400 cartridge (FIG. 9A, TEM (FIG. 9B, and ONi of EVs purified by ultracentrifugation from BAL of two subjects (FIG. 9C). We confirmed the presence of CD9, CD63, CCSP, and SFTPC on the surface membrane of BAL-EVs, which further validated that exh-EVs found in EBC originate from the biofluid that lines terminal bronchioles and alveoli.

Evaluating the Lung Tissue EV Surrogacy of Exh-EVs by miRNA NGS Analyses

We sought to determine whether the microRNA (miRNA) cargos of purified exh-EVs could be utilized as a surrogate measure for those of lung tissue EVs. Thus, for these experiments we collected biofluids from five different anatomic airway levels: mouth rinse, buccal brushing, bronchial brushing, bronchoalveolar lavage (BAL) and EBC from 18 subjects (Table 1) selected from a cohort of patients evaluated at the Montefiore Medical Center and undergoing bronchoscopy for clinical respiratory reasons.

TABLE 1
Demographics and clinical data on subjects (n = 18) recruited from the Montefiore Medical
Center (MMC), which are included for analysis of their 5 level of airway samples (i.e., mouth
rinse, buccal brush, Bronchial brush, Bronchoalveolar lavage (BAL) and exhaled breath condensates
(EBC) samples) in this study. On the right of the table are details on the samples that were collected
and evaluated (i.e., mouth wash (MW), buccal brush (BB), bronchial brush (BrB), bronchoalveolar
lavage (BAL), and exhaled breath condensates (EBC)) in small-RNA NGS libraries 1 through 7.

| Subject | | | Smoking | Pack | Quit | Underlying | Tumor | | |
|---|---|---|---|---|---|---|---|---|---|
| ID | Age | Gender | Status | Years | Years | Lung Disease | Diagnosis | Tumor | |
| 677 | [symbol] 3 | M | Current | 46 | 0 | None | Benign | — | MR/BB |
| 783 | 4 [symbol] | F | Never | 0 | 0 | Asthma | [symbol] /other | — | BrB |
| 111 [symbol] | 55 | F | Never | 0 | 0 | COPD | Benign | — | BAL/EBC |
| 1124 | [symbol] | F | Never | 0 | 0 | Non | [symbol] /other | — | Small-RNA |
| 12 [symbol] 3 | 28 | M | Never | 0 | 0 | None | Benign | — | NGS |
| 1350 | 67 | M | Curent | 30 | 16 | COPD | Squamous | IV | Librariws |
| 1353 | 58 | M | Never | 0 | 0 | None | Benign | — | 1-7 |
| 1354 | 59 | M | Current | 23.5 | 0 | COPD | Adeno | IIIA | |
| 1355 | 5 [symbol] | M | Never | 0 | 0 | None | [symbol] /other | — | |
| 1357 | 61 | M | Current | 30 | 0 | None | NSCLC | IIIB | |
| 1360 | 74 | F | Current | 59 | 0 | Astana, COPD | Small Cell | L | |
| 1362 | 61 | F | Never | 0 | 0 | None | Benign | — | |
| 1364 | 72 | M | Current | 116 | 0 | None | Squamous | IIA | |
| 1371 | 49 | M | Current | 1.8 | 0 | Bronchiectasis | Benign | — | |
| 1433 | 67 | M | Current | 12.25 | 0 | COPD | Squamous | IIB | |
| 1435 | 78 | F | Former | 5 [symbol] | 1 | COPD | Squamous | IIIA | |
| 1436 | 6 [symbol] | F | Never | 0 | 0 | None | Benign | — | |
| 1439 | 72 | F | Current | 29.5 | 0 | None | Adeno | IIIB | |

[symbol]

indicates data missing or illegible when filed

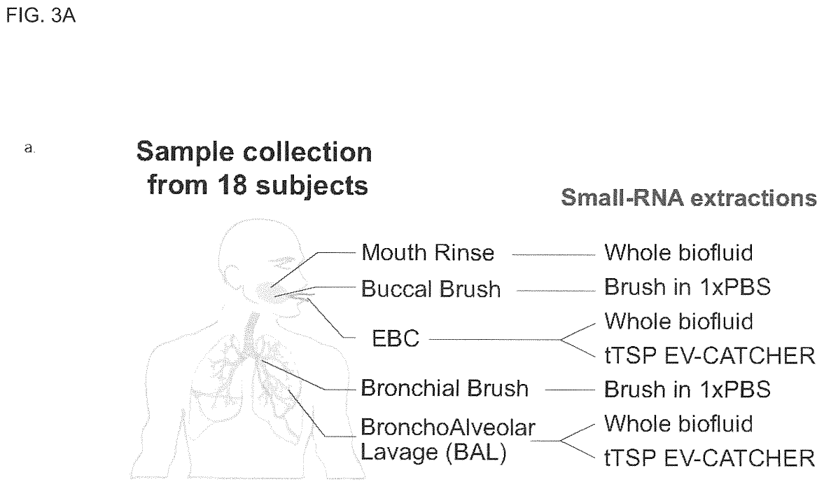

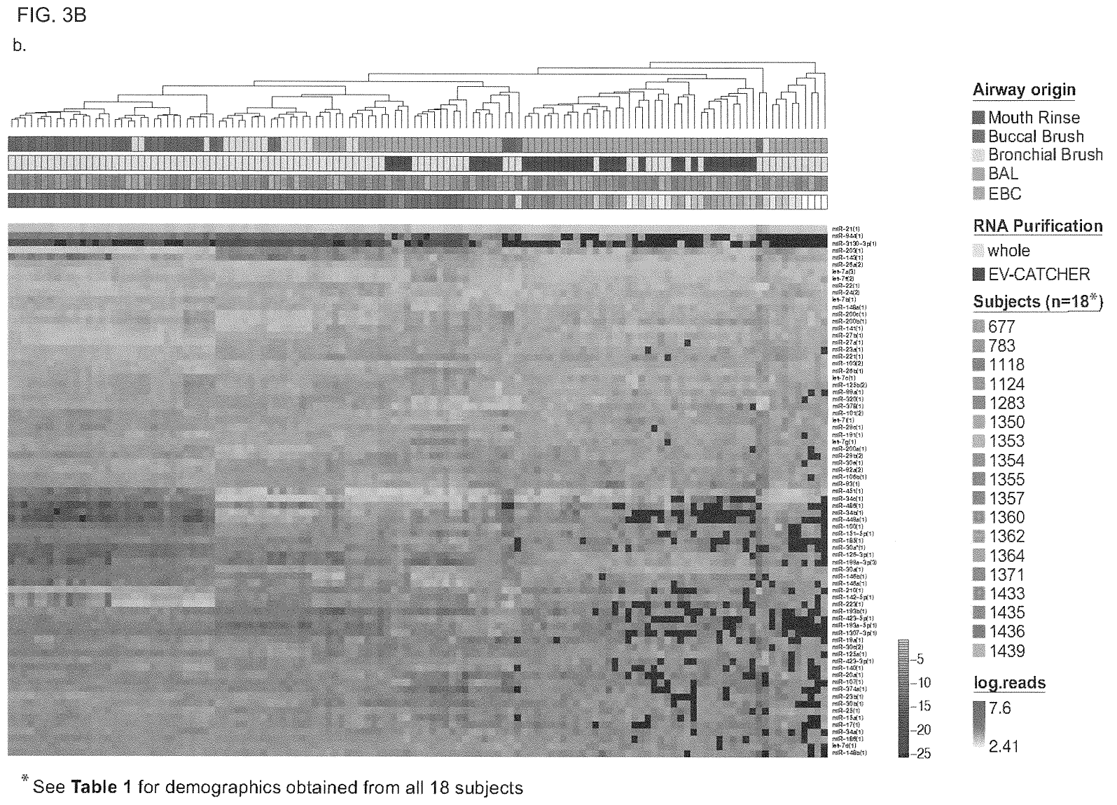

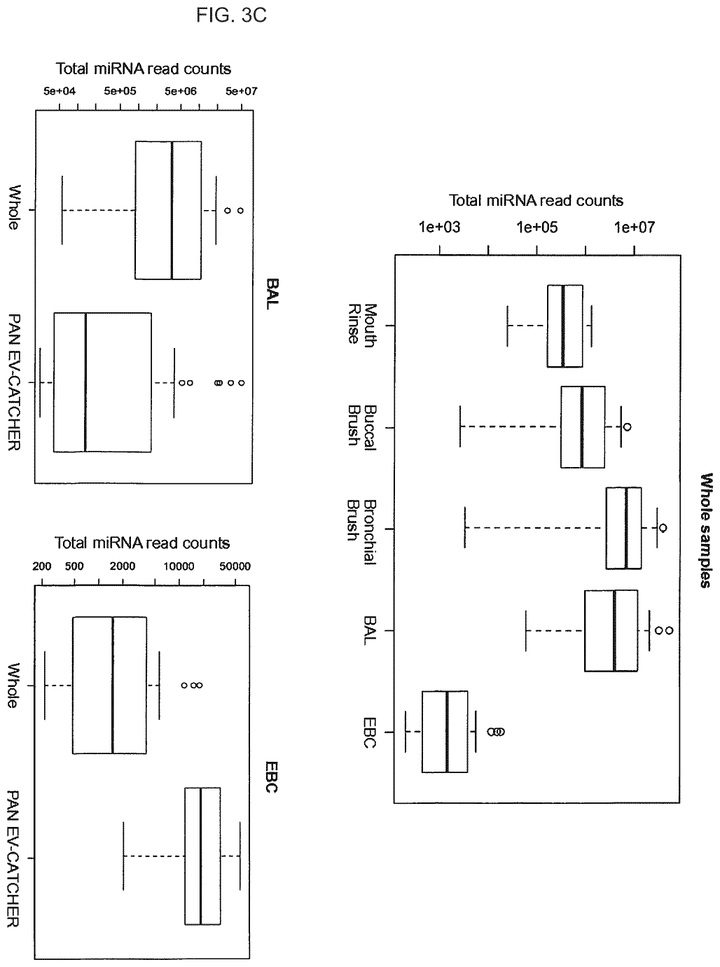

We performed small-RNA extractions from whole biofluids obtained from mouth rinse (200 pL), EBC (200 pL), BAL (200 pL), brushes obtained from buccal brush (200 pL) and bronchial brushing (200 pL), and from EVs purified using our human tTSP EV-CATCHER assay (anti-CD9/CD63/CD81) from matched EBC (200 pL) and BAL (200 pL) samples, as detailed in FIG. 3A. We then conducted small-RNA sequencing experiments of the different RNA samples and obtained miRNA expression profiles for the 18 subjects across all 5 anatomic airway levels. The miRNA data were analyzed, revealing that the miRNA profiles of samples obtained from the mouth and buccal brush clustered together (FIG. 3B heatmap, left), the miRNA profiles from bronchial brushing and whole BAL clustered together (FIG. 3B heatmap, center), and the miRNA profiles of EVs purified from BAL and exh-EVs purified from EBC clustered together (FIG. 3B, right). These results indicated that the miRNA contents of EBC and exh-EVs more closely recapitulated those of BAL-derived EVs that originate in the deep lung.

As we observed that miRNA profiles of whole EBC generally displayed a lower number of miRNA reads than miRNA profiles obtained from the other biofluids (FIG. 3B, see log.reads color gradient on heatmap), we evaluated the number of miRNA reads from each of the 5 different airway samples in FIG. 3C (upper panel). We observed that BAL, bronchial brushing, buccal brushing and mouth rinse all provided the largest number of miRNA reads, possibly due to larger RNA inputs from different biological sources (i.e., cell debris and EVs,). Then, we observed that NGS data from whole EBC samples contained 100- to 1,000-fold fewer miRNA read counts compared to the other four airway levels (FIG. 3C, upper panel). Because both BAL and EBC were analyzed as both whole biofluid fractions and EV fractions (i.e., purified using our tTSP EV-CATCHER), we compared the total number of miRNA reads between the whole biofluids the purified EV fractions (FIG. 3C, lower panels). As anticipated for BAL, small-RNA sequencing of the whole biofluid generated over a 100-fold higher number of miRNA reads than RNA purified from EVs isolated from BAL EVs (FIG. 3C, bottom left panel). However, to our surprise, we determined that for EBC the opposite was observed, with an average of 10-fold increase in the number of miRNAs reads for RNA purified from exh-EVs than for RNA purified from whole EBC (FIG. 3C, bottom right panel). These miRNA analyses indicate that the targeted isolation of exh-EV from EBC provides greater signal-to-noise ratios and thus enriches miRNA reads for NGS analyses.

Increasing miRNA Read Depth of Exh-EVs by Targeting Terminal Bronchiole and Alveoli Proteins Prior to miRNA NGS Analyses

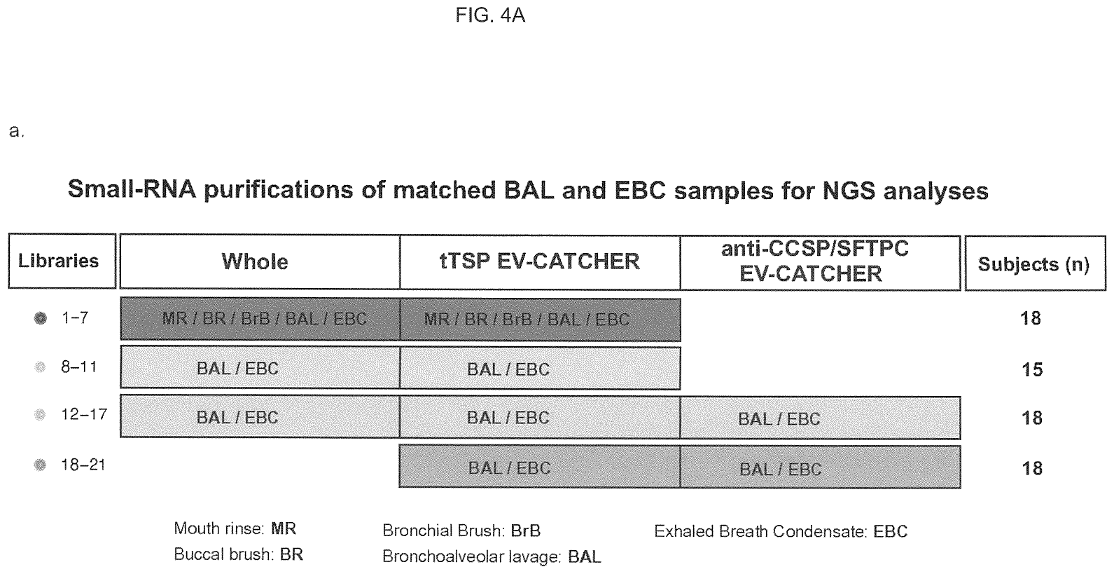

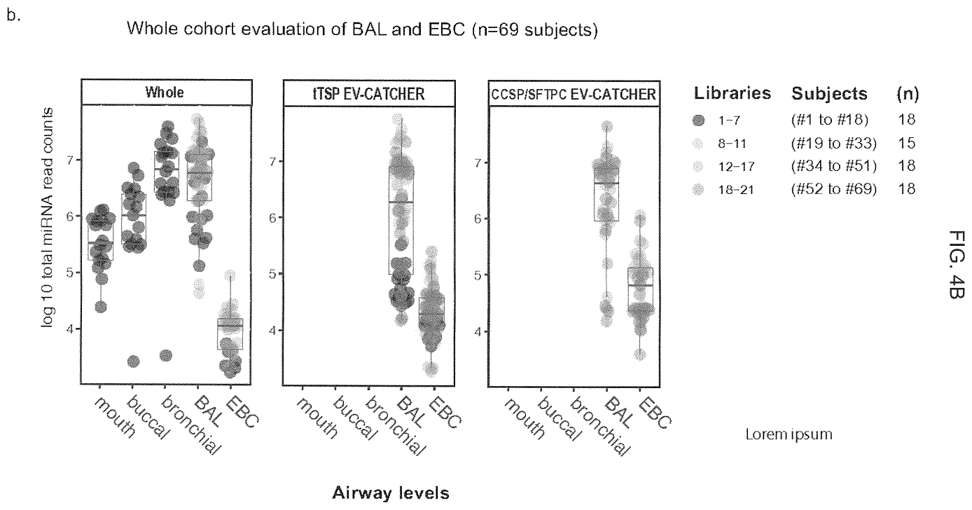

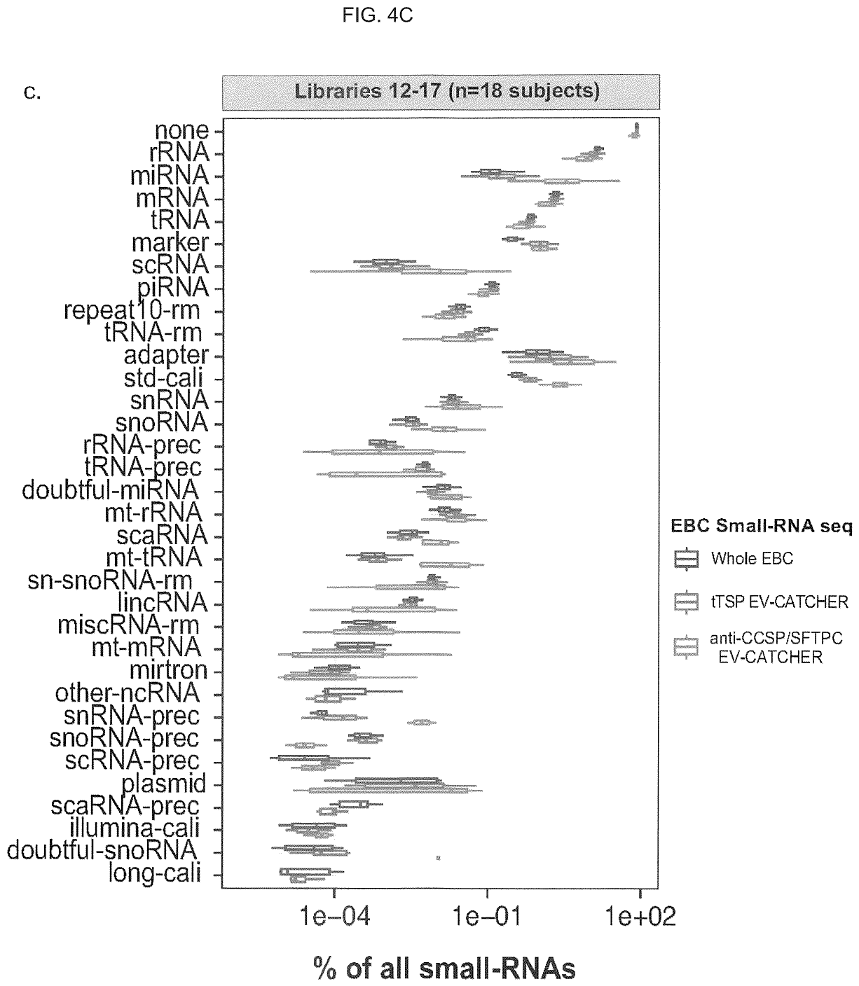

As depicted in FIG. 2A, FIG. 2B, FIG. 2C, and FIG. 2D, a population of exh-EVs contained within EBC harbors lung tissue-specific proteins, such as CCSP and SFTPC. Because small-RNA sequencing of exh-EVs purified with our tTSP EV-CATCHER assay from whole EBC enhanced the sequencing depth of miRNAs in small-RNA NGS libraries, we proceeded to investigate whether purifying exh-EVs of deep lung origin (i.e., positive for CSSP and SFTPC), would further improve the number of miRNA reads. For this purpose, we expanded our analyses from 18 subjects (Table 1) to 69 subjects (see demographic and clinical details on 51 additional subjects in Supplemental Table 1) and thus compared the total number of miRNA reads obtained from RNA extracted from matched BAL and EBC samples for all our small-RNA libraries (see sample distribution in different libraries included in this analysis detailed in FIG. 4A).

SUPPLEMENTAL TABLE 1
Demographics and clinical data from the additional 51 subjects
recruited at the Montefiore Medical Center (MMC), from whom matched
BAL and EBC were collected and analyzed in this study.

| | | | | | | [symbol] | | | |
|---|---|---|---|---|---|---|---|---|---|
| Subject | | | [symbol] | [symbol] | [symbol] | [symbol] | [symbol] | [symbol] | |
| ID | Age | Gender | [symbol] | [symbol] | [symbol] | [symbol] | [symbol] | [symbol] | |
| [symbol] | [symbol] | [symbol] | [symbol] | [symbol] | 0 | COPD | [symbol] | [symbol] | BAL/ [symbol] |
| [symbol] | [symbol] | [symbol] | [symbol] | [symbol] | 0 | [symbol] | [symbol] | [symbol] | [symbol] - [symbol] |
| [symbol] | [symbol] | [symbol] | [symbol] | [symbol] | 0 | [symbol] | [symbol] | [symbol] | [symbol] |
| [symbol] | [symbol] | [symbol] | [symbol] | [symbol] | 0 | COPD | [symbol] | [symbol] | [symbol] |
| [symbol] | [symbol] | [symbol] | [symbol] | [symbol] | 0 | [symbol] | [symbol] | [symbol] | [symbol] -11 |
| [symbol] | [symbol] | [symbol] | [symbol] | [symbol] | 0 | [symbol] | [symbol] | [symbol] | |
| [symbol] | [symbol] | [symbol] | [symbol] | [symbol] | 0 | [symbol] | [symbol] | — | |
| [symbol] | [symbol] | [symbol] | [symbol] | [symbol] | 0 | [symbol] | [symbol] | — | |
| [symbol] | [symbol] | [symbol] | [symbol] | [symbol] | 0 | [symbol] | [symbol] | — | |
| [symbol] | [symbol] | [symbol] | [symbol] | [symbol] | 0 | [symbol] | [symbol] | — | |
| [symbol] | [symbol] | [symbol] | [symbol] | [symbol] | 0 | COPD | [symbol] | — | |
| [symbol] | [symbol] | [symbol] | [symbol] | [symbol] | 0 | [symbol] | [symbol] | — | |
| [symbol] | [symbol] | [symbol] | [symbol] | [symbol] | 10 | [symbol] | [symbol] | — | |
| [symbol] | [symbol] | [symbol] | [symbol] | [symbol] | 0 | [symbol] | [symbol] | — | |
| [symbol] | [symbol] | [symbol] | [symbol] | [symbol] | 0 | [symbol] | [symbol] | — | |
| [symbol] | [symbol] | [symbol] | [symbol] | [symbol] | [symbol] | COPD | [symbol] | — | BAL/ [symbol] |
| [symbol] | [symbol] | [symbol] | [symbol] | [symbol] | [symbol] | [symbol] | [symbol] | — | [symbol] |
| [symbol] | [symbol] | [symbol] | [symbol] | [symbol] | [symbol] | [symbol] | — | — | [symbol] |
| [symbol] | [symbol] | [symbol] | [symbol] | 0 | 0 | [symbol] | [symbol] | — | [symbol] |
| [symbol] | [symbol] | [symbol] | [symbol] | 0 | 0 | [symbol] | — | — | 12-17 |
| [symbol] | [symbol] | [symbol] | [symbol] | [symbol] | [symbol] | [symbol] | — | — | |
| [symbol] | [symbol] | [symbol] | [symbol] | 0 | 0 | [symbol] | — | — | |
| [symbol] | [symbol] | [symbol] | [symbol] | [symbol] | [symbol] | [symbol] | — | — | |
| [symbol] | [symbol] | [symbol] | [symbol] | [symbol] | [symbol] | COPD | — | — | |
| [symbol] | [symbol] | [symbol] | [symbol] | [symbol] | 0 | [symbol] | — | — | |
| [symbol] | [symbol] | [symbol] | [symbol] | 0 | 0 | [symbol] | [symbol] | — | |
| [symbol] | [symbol] | [symbol] | [symbol] | [symbol] | [symbol] | COPD | — | — | |
| [symbol] | [symbol] | [symbol] | [symbol] | [symbol] | [symbol] | [symbol] | [symbol] | — | |
| [symbol] | [symbol] | [symbol] | [symbol] | — | [symbol] | [symbol] | — | — | |
| [symbol] | [symbol] | [symbol] | [symbol] | [symbol] | 0 | COPD | [symbol] | — | |
| [symbol] | [symbol] | [symbol] | [symbol] | [symbol] | 0 | [symbol] | — | — | |
| [symbol] | [symbol] | [symbol] | [symbol] | [symbol] | 0 | [symbol] | [symbol] | — | |
| [symbol] | [symbol] | [symbol] | [symbol] | [symbol] | 0 | [symbol] | [symbol] | — | |
| [symbol] | [symbol] | [symbol] | [symbol] | [symbol] | [symbol] | [symbol] | [symbol] | — | BAL/ [symbol] |
| [symbol] | [symbol] | [symbol] | [symbol] | [symbol] | 0 | [symbol] | [symbol] | — | [symbol] |
| [symbol] | [symbol] | [symbol] | [symbol] | 0 | 0 | [symbol] | [symbol] | — | [symbol] |
| [symbol] | [symbol] | [symbol] | [symbol] | 0 | 0 | [symbol] | [symbol] | — | [symbol] |
| [symbol] | [symbol] | [symbol] | [symbol] | 0 | 0 | [symbol] | [symbol] | — | 18-21 |
| [symbol] | [symbol] | [symbol] | [symbol] | [symbol] | 0 | [symbol] | [symbol] | — | |
| [symbol] | [symbol] | [symbol] | [symbol] | [symbol] | [symbol] | COPD | [symbol] | — | |
| [symbol] | [symbol] | [symbol] | [symbol] | [symbol] | [symbol] | [symbol] | [symbol] | — | |
| [symbol] | [symbol] | [symbol] | [symbol] | 0 | 0 | [symbol] | [symbol] | — | |
| [symbol] | [symbol] | [symbol] | [symbol] | 0 | 0 | [symbol] | [symbol] | — | |
| [symbol] | [symbol] | [symbol] | [symbol] | [symbol] | 2 | [symbol] | [symbol] | — | |
| [symbol] | [symbol] | [symbol] | [symbol] | [symbol] | 0 | [symbol] | [symbol] | — | |
| [symbol] | [symbol] | [symbol] | [symbol] | [symbol] | 0 | [symbol] | [symbol] | — | |
| [symbol] | [symbol] | [symbol] | [symbol] | 0 | 0 | [symbol] | [symbol] | — | |
| [symbol] | [symbol] | [symbol] | [symbol] | 0 | 0 | [symbol] | [symbol] | — | |
| [symbol] | [symbol] | [symbol] | [symbol] | [symbol] | 0 | [symbol] | [symbol] | — | |
| [symbol] | [symbol] | [symbol] | [symbol] | [symbol] | 0 | COPD | [symbol] | — | |
| [symbol] | [symbol] | [symbol] | [symbol] | [symbol] | 0 | [symbol] | [symbol] | — | |

[symbol]

indicates data missing or illegible when filed

As displayed on FIG. 4B, the total miRNA reads for whole BAL, BAL-EVs purified with our tTSP-EV-CATCHER or our anti-CCSP/SFTPC EV-CATCHER were overall within the same orders of magnitude, but generally higher for whole BAL samples (FIG. 4B). However, when we compared the total number of miRNA reads from whole EBC, exh-EVs purified using our tTSP EV-CATCHER assay, and exh-EVs purified with our anti-CCSP/SFTPC EV-CATCHER assay, we observed a steady increase in the number of miRNA reads for samples evaluated from the same subjects with all three methods (FIG. 4B, left, middle, and right, see samples from libraries 12-17 that underwent the three purification methods, light blue). We generally noted that the highest increase in the number of miRNA reads was for exh-EVs purified with our anti-CCSP/SFTPC EV-CATCHER assay, close to 100-fold when compared to whole EBC (FIG. 4B, left, middle, and right panels for libraries 8-11 (yellow), and 18-21 (green)).

In order to understand the reason for differences in the number of miRNA reads, we investigated the species content of the small-RNAs captured and sequenced from whole EBC, exh-EVs purified with our tTSP EV-CATCHER assay, and exh-EVs purified with our CCSP/SFTPC EV-CATCHER assay for small-RNA libraries 12 to 17 (see Supplemental Table 1), which included samples from 18 different subjects (FIG. 4C). Our analyses revealed that exh-EVs purified with the CCSP/SFTPC EV-CATCHER assay contained an increased number of miRNAs, scRNAs, snoRNAs, scaRNAs, mt-tRNA, and snRNA precursors, when compared to whole EBC and exh-EVs purified with the tTSP EV-CATCHER assay. Importantly, we noted that rRNAs, mRNAs, tRNAs, piRNAs, tRNA-rm, lincRNA, and other non-coding RNAs (ncRNAs) were more abundant in the small-RNA libraries prepared using RNA extracted from whole EBC and from exh-EVs purified with the tTSP EV-CATCHER assay. These experiments validated that exh-EVs, which harbor lung tissue terminal bronchiole and alveoli secretory proteins CCSP and SFTPC, were enriched with miRNA transcripts when compared to whole EBC.

Evaluating miRNA Surrogacy of Exhaled EVs for BAL EVs for Subjects Classified by Smoking, Asthma, and Lung Cancer Status

Next, we examined whether the miRNA expression profiles of exh-EVs were representative of those BAL-derived EVs when evaluating the smoking, asthma, or lung cancer status of the subjects with available and analyzed matched BAL and EBC samples.

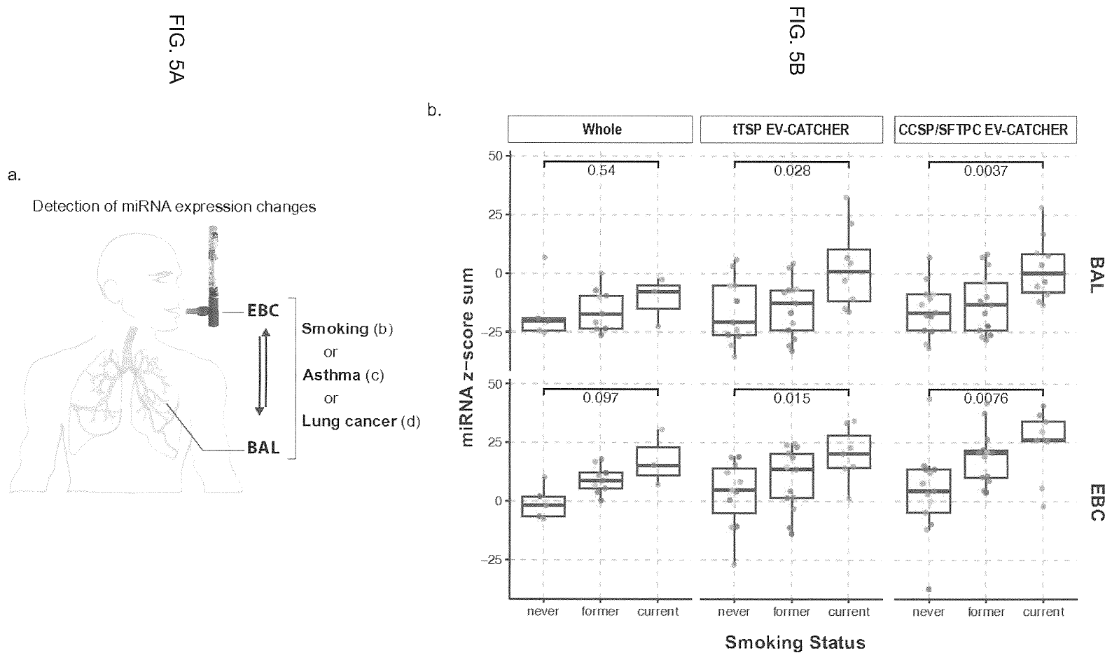

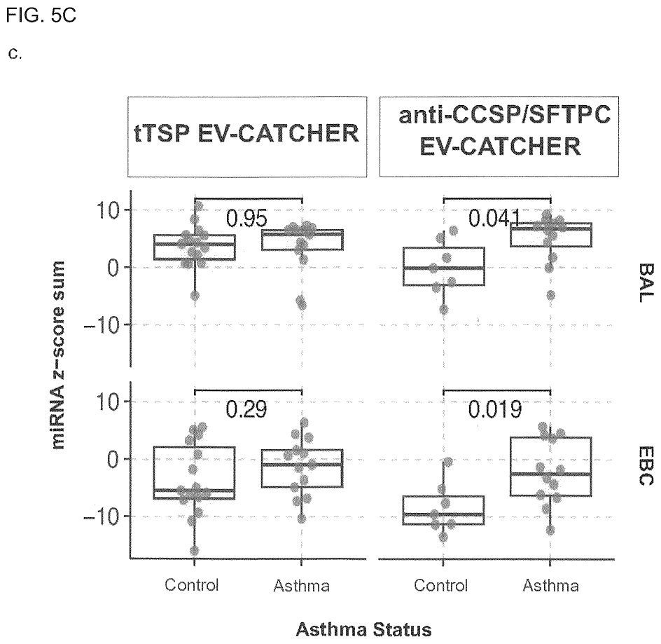

Considering that smoking permanently affects the lung epithelium of a subject by altering miRNA expression in lung epithelial cells and the EVs they secrete in BAL [85], we evaluated whether miRNA expression differences in BAL-EVs of never, former and current smokers could also globally be detected in exh-EVs purified from matched EBC samples. We focused our analyses on miRNA NGS data obtained from libraries 12-17 and 18-21 (see Supplemental Table 1), which included: (i) subjects whose smoking status was annotated as never (n=13), former (n=13), and current smokers (n=10); (ii) matched BAL and EBC samples from the same subjects; and (iii) RNA samples extracted from whole biofluid, EVs purified with our tTSP and anti-CCSP/SFTPC EV-CATCHER assays from matched BAL and EBC samples (FIG. 5A). As displayed in FIG. 5B, we calculated the miRNA z-score for the 317miRNAs consistently detected between matched whole BAL and whole EBC. Each individual miRNA's z-value was added or subtracted to obtain a z-score for each subject in each of the three smoking groups (i.e, never, former, and current smokers). For whole BAL and whole EBC, we observed similar increasing miRNA expression trends between never and current smokers, but the corresponding p-values did not reach significance (FIG. 5B, upper and lower left panels, with p-values of 0.57 for whole BAL and 0.097 for whole EBC). However, when calculating the z-score for miRNAs sequenced from EVs captured with our tTSP EV-CATCHER assay from matched BAL and EBC samples, we observed a similar and significant miRNA expression increasing trend between never and current smokers (FIG. 5B, upper and lower middle panels, with p-values of 0.028 for BAL and 0.015 for EBC). Finally, when comparing the z-scores of miRNAs sequenced from EVs purified with our anti-CCSP/SFTPC EV-CATCHER assay from matched BAL and EBC samples, we observed an even greater significant miRNA increasing expression trend between never and current smokers (FIG. 5B, upper and lower right panels, with p-values of 0.0037 for BAL and 0.0076 for EBC). These results indicated that miRNA expression changes detected in BAL could also be detected in matched EBC samples, but that greater sensitivity may be achieved when analyzing the miRNA profiles of EVs selectively purified with our anti-CCSP/SFTPC EV-CATCHER assay.

Similarly, we evaluated whether miRNA expression differences detectable in BAL EVs of subjects diagnosed with asthma, compared to subjects without asthma, could also be detected in exh-EVs. Since our analyses repeatedly demonstrated that we achieved increased miRNA reads (FIG. 4B and FIG. 4C) and more significant miRNA expression differences (FIG. 5B) by evaluating EVs rather than whole biofluids (i.e., BAL or EBC), we concentrated these analyses on EVs purified with our tTSP and anti-CCSP/SFTPC EV-CATCHER assays from matched EBC and BAL samples of control subjects (i.e., without asthma) and patients diagnosed with asthma (FIG. 5C). We determined that significant miRNA expression differences could not be detected in EVs purified from BAL (p-val<0.95) or exh-EVs purified from matched EBC samples (p-val<0.29) using our tTSP EV-CATCHER assay between control subjects without asthma and patients diagnosed with asthma (samples analyzed in libraries 8-21, See Supplemental Table 1). However, when globally evaluating miRNA z-score differences between EVs purified from BAL samples purified from control subjects and asthma patients, we observed a statistical significance (p-val<0.041), which was similarly observed with the analysis of the same miRNAs in exh-EVs purified from matched EBC samples (p-val<0.019) with our anti-CCSP/SFTPC EV-CATCHER assay from control subjects with asthma and subjects diagnosed with asthma (samples analyzed in libraries 12-21, See Supplemental Table 1). Finally, we examined whether significant miRNA expression differences (i.e., miRNA z-score) could similarly be detected between EVs purified from matched BAL and EBC samples, between subjects with benign lung lesions and patients with confirmed lung tumors (FIG. 5D, samples analyzed in libraries 12-17 and 18-21, See Supplemental Table 1). Our analyses identified miRNA z-scores displaying that significantly differentially expressed miRNAs detected in EVs purified from BAL (P-value<0.022) could also be detected in exh-EVs purified from matched EBC samples with our tTSP EV-CATCHER assay, between patients with benign lung lesions and patients with confirmed lung tumors (FIG. 5D). We similarly detected more significant expression differences for the selected miRNAs between the two groups, for EVs purified from BAL (p-val<0.02) and exh-EVs purified from matched EBC samples (p-val<0.0094) with our anti-CCSP/SFTPC EV-CATCHER assay. Altogether these analyses support the conclusion that miRNA expression differences detected in BAL EVs based on the smoking, asthma, and lung tumor status of subjects can also be detected in exh-EVs purified from matched EBC samples, and that BAL EVs and exh-EVs purified with our anti-CCSP/SFTPC EV-CATCHER assay contain miRNAs with greater discriminatory power.

Evaluating the miRNA Expression Profiles of Exh-EVs Purified with Our tTSP and Anti-CCSP/SFTPC EV-CATCHER Assays Based on Lung Cancer Status

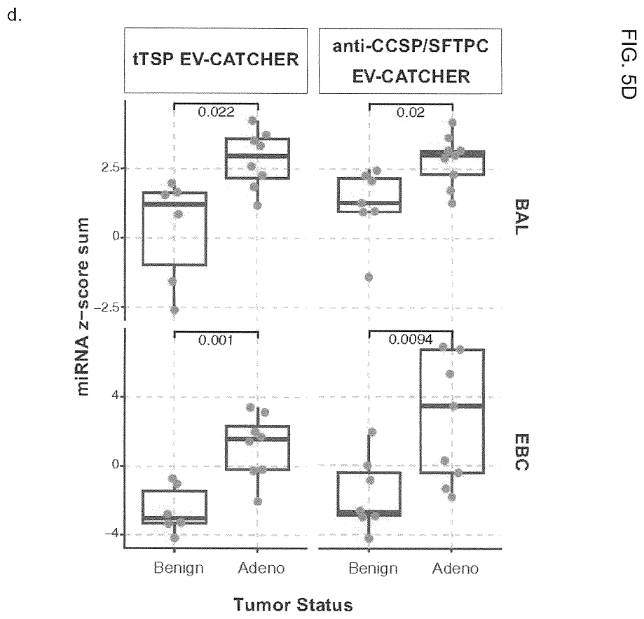

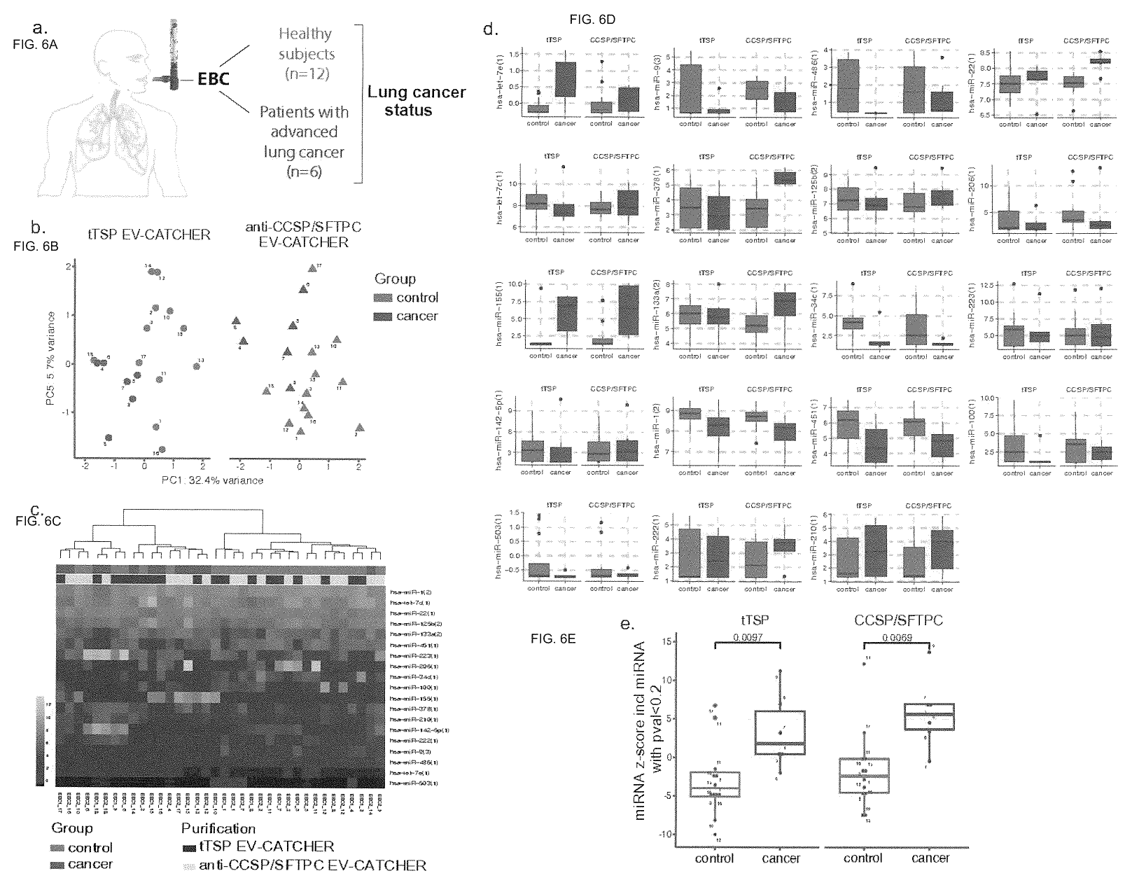

a. As we demonstrated the surrogacy of EBC exh-EVs for BAL EVs, and conducted all of our analyses on EBC samples collected at the Montefiore Medical Center, Bronx NY (MMC; 69 subjects from Table 1 and Supplemental Table 1), we sought to evaluate the robustness of our EV-CATCHER assays for targeted purification of exh-EVs from EBC samples collected (also with the RTube™ device, FIG. 6A) at a different medical center, the Hackensack University Medical Center, Hackensack NJ (HUMC). As displayed in Table 2, for these analyses, we selected EBC samples that were collected from a group of non-smoking young healthy adults (n=12), and from a small group of patients diagnosed with advanced bronchogenic carcinoma (n=6; adenocarcinoma (NSCLC, n=4), squamous cell carcinoma (SCLC; n=1), and large cell carcinoma (a rare type of NSCLC; n=1)).

TABLE 2
Demographics and clinical data on subjects (n = 16) who
were recruited at the Hackensack University Medical Center
(HUMC) for sole collection of EBC. The subjects include 12
healthy non-smoking adults and 6 patients diagnosed with stage
IV squamous cell lung carcinoma (n = 1, SCLC), large
cell lung carcinoma (n = 1, a rare form of NSCLC), and
non-small cell lung carcinoma (n = 4, NSCLC).

| Con- | | | | | | | |
|---|---|---|---|---|---|---|---|
| trols | Gender | Age | Cases | Gender | Age | Cancer | Stage |
| 1 | F | 38 | 1 | M | 70 | Sq. CLC | Stage IV |
| 2 | F | 26 | 2 | M | 55 | Lg. CLC | Stage IV B |
| 3 | M | 34 | 3 | F | 75 | Adeno. | Stage IV A |
| 4 | F | 31 | 4 | F | 57 | Adeno. | Stage IV |
| 5 | F | 31 | 5 | M | 84 | Adeno. | Stage IV A |
| 6 | F | 25 | 6 | M | 81 | Adeno. | Stage IV |
| 7 | F | 44 | — | | | | |
| 8 | F | 30 | — | | | | |
| 9 | M | 50 | — | | | | |
| 10 | F | 39 | — | | | | |
| 11 | M | 19 | — | | | | |
| 12 | F | 35 | — | | | | |

We selectively chose to analyze EBC samples from subjects at different ends of the health spectrum (i.e., healthy controls vs lung cancer patients) to ensure that differentially expressed miRNAs could be detectable. As shown in FIG. 6B, Principal Component Analysis (PCA) plots revealed apparent differential miRNA expression between our two groups (control (healthy) vs cancer), for exh-EVs purified by both our tTSP and anti-CCSP/SFTPC EV-CATCHER assays. When evaluating the top 19 differentially expressed exh-EV miRNAs (P-value<0.05) commonly identified by both EV-CATCHER purification assays (FIG. 6C), we observed that EBC samples from healthy controls formed a distinct heat map cluster, while EBC samples from patients diagnosed with advanced lung cancer predominantly belonged to another heat map cluster. Next, when comparing the differential expression of these 19 miRNAs in individual box plots (FIG. 6D), between the two groups (healthy controls vs lung cancer patients) and for each purification method (tTSP vs anti-CCSP/SFTPC EV-CATCHER assays), we determined that these miRNAs generally displayed the same differential expression trends, albeit with varying levels of significance. Specifically, we found that let-7e and miR-155 were more upregulated, while miR-9, miR-486, miR-34c, miR-206, miR-100, and miR-503 were more downregulated in exh-EVs purified with our tTSP EV-CATCHER assay than exh-EVs purified with our anti-CCSP/SFTPC EV-CATCHER assay (FIG. 6D). Comparatively, we found that miR-22, miR-378, miR-125b, miR-133a, miR-222, and miR-210 were more upregulated, and miR-206, miR-34c, miR-1, and miR-451 more downregulated in exh-EVs purified with our anti-CCSP/SFTPC than our tTSP EV-CATCHER assays (FIG. 6D). When we computed a group-related z-score of the top 14 (out of 19) most differentially expressed miRNAs, we observed that the miRNA analysis of exh-EVs purified with the anti-CCSP/SFTPC EV-CATCHER assay offered a slightly superior discriminatory power (p-value of <0.0069) than that of exh-EVs purified with the tTSP EV-CATCHER assay (with a p-value of <0.0097).

Altogether, these small-RNA NGS analyses confirmed the robustness of our EV-CATCHER assay and suggest that tissue-specific enrichment of exh-EVs from EBC may help identify miRNAs whose deregulated expression correlates with unique lung pathologies.

### Discussion

In this study we evaluated and confirmed the presence of extracellular vesicles (EVs) in exhaled breath condensates (EBC). We analyzed their miRNA profiles and found a high degree of correlation with those purified from bronchoalveolar lavages (BAL). Using super resolution nanoimaging we demonstrated that exh-EVs harbor unique surface proteins of terminal bronchiole and alveoli origin (i.e., Clara Cell Specific Protein (CCSP) and Surfactant Protein C (SFTPC)), which we then leveraged for the enrichment of exh-EVs from EBC using our customizable EV-CATCHER assay. Our comparative miRNA expression analyses confirmed the surrogacy of exh-EVs purified from EBC for those similarly purified from matched BAL samples Using the anti-CCSP/SFTPC EV-CATCHER assay further validated the lung tissue origin of exh-EVs and that subjects may be distinguished based of their lung pathologies.

Given the limited number of reports describing the detection and the characteristics of EVs in EBC to date, we initially sought to establish whether current molecular validation standards (i.e., MISEV2018 guidelines; [5,6,86]) could be effectively used to confirm the presence of EVs in EBC samples. We used microfluidic resistive pulse sensing (MRPS) and TEM to characterize exhaled EVs (exh-EVs), and Western blot analyses to validate the presence of tetraspanins (i.e., CD63, CD9, CD81) on purified exh-EVs, which we combined with ONi super resolution nanoimaging analyses to confirm the surface localization of these proteins. Importantly, we determined that our EV purification methods (i.e., ultracentrifugation and EV-CATCHER assay) yielded no detectable levels of albumin and ApoB, and only trace amounts of ApoA1, all of which are frequently encountered as contaminants in blood samples, but not for EBC. Our quantitation also revealed that although exh-EVs were detectable and purifiable from EBC, they were in low abundance and thus required a significant concentration step for subsequent evaluations. However, when using established EV purification techniques, including ultracentrifugation followed by immuno-purification using our customizable non-magnetic bead-based EV-CATCHER assay, we could effectively isolate and enrich for exh-EVs from EBC.

Next, to evaluate the biological utility of exh-EVs for the non-invasive sampling of the lung, we focused our analyses on biological markers uniquely found to be uniquely expressed by terminal bronchiole and alveoli cells. Specifically, we selected bronchiolar Clara cells because they express a unique lung protein (i.e., Clara Cell Specific Protein (CCSP)) and they have been implicated in lung cancer initiation [87-89]. CCSP is a 15.8-kDa homodimeric protein with a central hydrophobic region, which is uniquely secreted in large amounts by non-ciliated terminal bronchiolar Clara cells to protect the respiratory tract from stress (i.e., oxidative, pathogenic and inflammatory) [90,91]. Importantly, Wang et al. determined that although it is a secreted protein, it can also be localized in the membrane of Clara cells [92,93], and they suggested that the central hydrophobic region of CCSP [94] may allow for it to be trapped within the cytoplasmic membrane during its secretion. Then, we selected Alveolar type II (ATII) cells because they have also been implicated in lung cancer initiation and because they uniquely secrete SFTPC, a 21 kDa transmembrane proprotein (proSPC) that is processed through a sequence of proteolytic cleavages into a 3.7 kDa secretory protein before it is released from the cytoplasmic membrane, which has previously been detected on the surface of ATII cells cultured in vitro [95-98]. Additionally, a recent study by Choudhary et al. (2021) also revealed that EVs produced by ATII cells contained SFTPC [99]. As both CCSP and SFTPC have been localized in the membrane of their respective secretory cells, we hypothesized that they may also be present on the surface of exh-EVs and potentially be used for immuno-purification of exh-EVs using our customizable EV-CATCHER assay. Using super resolution nanoimaging (ONi), we confirmed that CCSP [94] and SFTPC [100] could be detected on the surface of exh-EVs. Our data was consistent between exh-EVs purified by ultracentrifugation alone or in combination with our EV-CATCHER assay customized with either tTSP (i.e., triple tetraspanin; CD9/CD63/CD81) or monoclonal antibodies directly targeting the CCSP and SFTPC proteins. To our knowledge, this is the first such demonstration, as to date no published studies have revealed the localization of these two terminal bronchiole and alveolar cell-specific secretory proteins on the surface of EBC-derived exh-EVs. Furthermore, our immuno-purification analyses, using our customized EV-CATCHER assay, revealed that by targeting these two cell-specific secretory proteins, we could enrich EVs of lung tissue origin, which could also be found in BAL [101-104]. Biologically, the presence of secretory proteins on the surface of exh-EVs may suggest a function for binding a variety of particles, bacteria, and or viruses, prior to being aerosolized, within the biofluid lining the lungs. This hypothesis would align with recent findings, where we and others determined that circulating EVs detected in serum after viral infection harbor surface proteins (i.e., ACE-2) that can neutralize viruses (i.e., SARS-CoV-2) and thus contribute to the hosts immune defense [80,105].

Next, to further validate the surrogacy of exh-EV miRNA cargos for EVs of lung tissue origin, we conducted comprehensive miRNA expression profiling analyses of airway specimens collected at 5 different anatomic levels (i.e., mouth rinse, buccal brush, bronchial brush, BAL, and EBC) from 18 subjects recruited at the Montefiore Medical Center, Bronx NY (MMC). We also used matched BAL and EBC samples from another 51 subjects from our MMC cohort to further confirm the robustness of our findings. All our EBC samples were uniformly collected using the FDA registered RTube™ device, and our analyses produced the following results. First, we showed that the miRNA expression profiles obtained from whole EBC and exh-EVs purified with our tTSP EV-CATCHER assay clustered closely with those of whole BAL and BAL EVs (also purified with our tTSP EV-CATCHER assay), but not with those of other airway specimens. Second, miRNA NGS analyses of whole EBC, exh-EVs purified with our tTSP EV-CATCHER assay, and exh-EVs purified with our CCSP/SFTPC EV-CATCHER assay from the same subjects showed that enriching exh-EVs prior to conducting miRNA NGS analyses significantly increased the number of miRNA reads by up to 100-fold (i.e., when compared to miRNA NGS analyses of whole EBC). This increase in miRNA reads was correlated with a decrease in ribosomal RNAs, tRNAs, and other non-coding RNAs, which were less represented in exh-EVs, particularly those harboring proteins (i.e., CCSP and SFTPC) of terminal bronchiole and alveoli cellular origin. Third, we demonstrated the potential of exh-EV miRNAs to serve as surrogates for those of BAL EVs in relation to smoking, asthma, and lung cancer status. Consistent with reports that smoking is associated with permanent miRNA expression changes in epithelial lung cells and their secreted EVs [106-109 85], we observed that miRNA expression differences that were significantly detected in BAL EVs (i.e., especially those purified with the anti-CCSP/SFTPC EV-CATCHER assay) between never and current smokers could similarly be detected in exh-EVs purified from EBC with the same EV-CATCHER assays (i.e., tTSP and anti-CCSP/SFTPC EV-CATCHER). Similarly, miRNA analyses conducted on matched BAL EVs and EBC exh-EVs obtained from patients diagnosed with asthma or lung tumors (i.e., in two separate analyses) further determined that similar miRNA expression changes can be detected between BAL EVs and exh-EVs, with the greatest discriminatory power provided by EVs purified with the anti-CCSP/SFTPC EV-CATCHER assay. Although these analyses were conducted on small subject/patient groups and are thus too preliminary to identify any miRNA biomarkers associated with specific lung conditions (i.e., no validation of these findings), they indicate that the non-invasive collection of EBC and miRNA analysis of lung cell-specific exh-EVs has potential to correlate with clinical and diagnostic information and should be further investigated.

Finally, as we had conducted all our experimental analyses on EBC samples collected from the same medical center (i.e., the MMC), we sought to evaluate whether our optimized molecular assays (i.e., exh-EV purification and NGS analyses) could be utilized on a different set of EBC samples collected at another medical center (i.e., Hackensack University Medical Center), using the same RTube™ device. Because the collection of BAL is invasive, our additional analyses were conducted using EBC-only samples, which were obtained from healthy subjects (n=12) and a small group of patients diagnosed with advanced (i.e., stage IV) lung cancer (n=6). For these analyses, we compared the identification of differentially expressed miRNAs between exh-EVs purified with our tTSP EV-CATCHER assay and those purified with our CCSP/SFTPC EV-CATCHER assay for our two groups to evaluate the robustness of our small-RNA NGS detection. Our comparative analyses robustly identified similarly differentially expressed miRNAs between exh-EVs purified by both EV purification methods. Furthermore, as observed with miRNA analyses conducted using exh-EVs purified from EBC of non-smokers vs smokers, subjects without asthma vs patients with asthma, and patients with or without lung tumors from MMC, we found that exh-EVs enriched using our the anti-CCSP/SFTPC EV-CATCHER assay identified more significantly differentially expressed miRNAs than those purified with our tTSP EV-CATCHER assay. Although these experiments were not sufficiently powered to identify exh-EV miRNA biomarkers or miRNA signatures for the detection of lung cancer, and will require validation, we found that the top 3 differentially expressed miRNAs (i.e., miR-22, miR-155, miR-34c) detectable in exh-EVs purified by our two methods (i.e., tTSP and anti-CCSP/SFTPC EV-CATCHER assays) between healthy controls and patients diagnosed with stage IV lung cancer had previously been described for their involvement in inflammation (i.e., miR-155), the regulation of cellular proliferation (miR? [110]), epithelial-to-mesenchymal transition (miR? [111]), and cellular migration in lung cancers (miR? [112,113]). Interestingly, we observed that we could detect specific miRNAs (i.e., miR-378, miR-22) were significantly and consistently more differentially expressed in exh-EVs purified with the anti-CCSP/SFTPC EV-CATCHER assay customized for selective purification of Clara and Alveolar type II cells exh-EVs from the bulk of exh-EVs in EBC. These findings are preliminary, and we anticipate that additional and properly powered studies will be needed and will need to include EBC specimens from healthy adult subjects, subjects with history of lung disease and/or smoking, and patients with different types of lung cancers and at different stages. Such study will need to be conducted to fully evaluate the potential of quantifying exh-EV miRNAs for non-invasive detection of lung cancer, especially in comparison to existing technologies (i.e., CT scan) and state-of-the-art molecular detection assays to evaluate the sensitivity and reproducibility of this non-invasive approach. Based on this study, however, our findings suggest that stratifying exh-EVs from EBC based on their cellular origin, by using our customized EV-CATCHER assay or other ultra-sensitive immuno-purification assays, may enable detection of precise miRNA expression signatures associated with pathological lung cellular changes, but it will require further investigation. As EBC contains EVs of lung tissue origin, the analysis of exh-EVs offers a unique opportunity for discovery of lung-only biomarkers, whereas EVs that are released from the lung and that circulate in blood may be difficult to purify, due to their mixing with other organ EVs potentially harboring surface proteins similarly expressed in other cell-types outside of the lung. For example, studies have showed that CCSP, although enriched in lung tumors, can also be detected in prostate and endometrial cancers [114,115], which would preclude the sole purification of Clara cell EVs from blood and may provide false lung cancer positive signals in patients with other types of cancers. Nevertheless, in order to refine the selective purification of exh-EVs, especially those associated with cells involved in lung diseases, we propose that proteomic analyses of exh-EVs purified from subjects diagnosed with these specific lung diseases may help identify unique lung disease-related surface markers to further enhance enrichment of disease-related exh-EVs and improve the discriminatory power of their biomarkers for the detection of these lung diseases.

In summary, our analyses confirm the utility of our customizable EV-CATCHER assay for the selective purification of exh-EVs harboring surface proteins of terminal bronchiole and alveoli lung tissue origin from EBC. However, it is important to note that although the collection of EBC is noninvasive, generally rapid, productive (up to 2 ml of biofluid within 10 minutes), and inexpensive, it is currently not performed in standard practice. Unfortunately, there are currently no biobanks with annotated EBC samples from which specimens may be selected for analyses. Instead, the collection of EBC has to be done prospectively, and it will require time to assemble large enough cohorts to power studies that will evaluate the potential of exh-EVs miRNAs for non-invasive detection of different lung diseases, particularly lung cancer. Ultimately, the accurate distinction between benign and malignant lung nodules, which often cannot be made by CT imaging and thus requires biopsy, is the benchmark of sensitivity that putative exh-EV miRNA lung cancer biomarkers will have to achieve.

### REFERENCES

1. GBD 2019 Chronic Respiratory Diseases Collaborators. Global burden of chronic respiratory diseases and risk factors, 1990-2019: an update from the Global Burden of Disease Study 2019. EClinicalMedicine. 2023 May; 59:101936 doi: 10.1016/j.eclinm.2023.101936. PMID: 37229504; PMCID: PMC7614570
2. Safiri S, Carson-Chahhoud K, Noori M, Nejadghaderi S A, Sullman M J M, Ahmadian Heris J, Ansarin K, Mansournia M A, Collins G S, Kolahi A A, Kaufman J S. Burden of chronic obstructive pulmonary disease and its attributable risk factors in 204 countries and territories, 1990-2019: results from the Global Burden of Disease Study 2019. BMJ. 2022 Jul. 27; 378:e069679. doi: 10.1136/bmj-2021-069679. PMID: 35896191; PMCID: PMC9326843.
3. Safiri S, Carson-Chahhoud K, Karamzad N, Sullman M J M, Nejadghaderi S A, Taghizadieh A, Bell A W, Kolahi A A, Ansarin K, Mansournia M A, Collins G S, Kaufman J S. Prevalence, Deaths, and Disability-Adjusted Life-Years Due to Asthma and Its Attributable Risk Factors in 204 Countries and Territories, 1990-2019. Chest. 2022 February; 161(2):318-329. doi: 10.1016/j.chest.2021.09.042. Epub 2021 Oct. 23. PMID: 34699773.
4. Sung H, Ferlay J, Siegel R L, Laversanne M, Soerjomataram I, Jemal A, Bray F. Global Cancer Statistics 2020: GLOBOCAN Estimates of Incidence and Mortality Worldwide for 36 Cancers in 185 Countries. C A Cancer J Clin. 2021 May; 71(3):209-249. doi: 10.3322/caac.21660. Epub 2021 Feb. 4. PMID: 33538338.
5. Siegel R L, Miller K D, Fuchs H E, Jemal A. Cancer Statistics, 2021. C A Cancer J Clin. 2021 January; 71(1):7-33. doi: 10.3322/caac.21654. Epub 2021 Jan. 12. Erratum in: C A Cancer J Clin. 2021 July; 71(4):359. PMID: 33433946.
6. Barta J A, Powell C A, Wisnivesky J P. Global Epidemiology of Lung Cancer. Ann Glob Health. 2019 Jan. 22; 85(1):8. doi: 10.5334/aogh.2419. PMID: 30741509; PMCID: PMC6724220.
7. Ferlay J, Colombet M, Soerjomataram I, Parkin D M, Pineros M, Znaor A, Bray F. Cancer statistics for the year 2020: An overview. Int J Cancer. 2021 Apr. 5. doi: 10.1002/ijc.33588. Epub ahead of print. PMID: 33818764.
8. Nicholson A G, Tsao M S, Beasley M B, Borczuk A C, Brambilla E, Cooper W A, Dacic S, Jain D, Kerr K M, Lantuejoul S, Noguchi M, Papotti M, Rekhtman N, Scagliotti G, van Schil P, Sholl L, Yatabe Y, Yoshida A, Travis W D. The 2021 WHO Classification of Lung Tumors: Impact of Advances Since 2015. J Thorac Oncol. 2022 March; 17(3):362-387. doi: 10.1016/j.jtho.2021.11.003. Epub 2021 Nov. 20. PMID: 34808341.
9. Haddad D N, Sandler K L, Henderson L M, Rivera M P, Aldrich M C. Disparities in Lung Cancer Screening: A Review. Ann Am Thorac Soc. 2020 April; 17(4):399-405. doi: 10.1513/AnnalsATS.201907-556CME. PMID: 32017612; PMCID: PMC7175982.
10. Blandin Knight S, Crosbie P A, Balata H, Chudziak J, Hussell T, Dive C. Progress and prospects of early detection in lung cancer. Open Biol. 2017 September; 7(9):170070. doi: 10.1098/rsob.170070. PMID: 28878044; PMCID: PMC5627048.
11. Kanwal M, Ding X J, Cao Y. Familial risk for lung cancer. Oncol Lett. 2017 February; 13(2):535-542. doi: 10.3892/ol.2016.5518. Epub 2016 Dec. 20. PMID: 28356926; PMCID: PMC5351216.
12. Shankar A, Dubey A, Saini D, Singh M, Prasad C P, Roy S, Bharati S J, Rinki M, Singh N, Seth T, Khanna M, Sethi N, Kumar S, Sirohi B, Mohan A, Guleria R, Rath G K. Environmental and occupational determinants of lung cancer. Transl Lung Cancer Res. 2019 May; 8(Suppl 1):S31-S49. doi: 10.21037/tlcr.2019.03.05. PMID: 31211104; PMCID: PMC6546634.
13. Yu F, Xiao R, Li X, Hu Z, Cai L, He F. Combined effects of lung disease history, environmental exposures, and family history of lung cancer to susceptibility of lung cancer in Chinese non-smokers. Respir Res. 2021 Jul. 23; 22(1):210. doi: 10.1186/s12931-02101802-z. PMID: 34301263; PMCID: PMC8306005.
14. Corner J, Hopkinson J, Fitzsimmons D, Barclay S, Muers M. Is late diagnosis of lung cancer inevitable? Interview study of patients' recollections of symptoms before diagnosis. Thorax. 2005 April; 60(4):314-9. doi: 10.1136/thx.2004.029264. PMID: 15790987; PMCID: PMC1747353.
15. Van Hal G, Diab Garcia P. Lung cancer screening: targeting the hard to reach-a review. Transl Lung Cancer Res. 2021 May; 10(5):2309-2322. doi: 10.21037/tlcr-20-525. PMID: 34164279; PMCID: PMC8182716.
16. Billatos E, Vick J L, Lenburg M E, Spira A E. The Airway Transcriptome as a Biomarker for Early Lung Cancer Detection. Clin Cancer Res. 2018 Jul. 1; 24(13):2984-2992. doi: 10.1158/1078-0432.CCR-16-3187. Epub 2018 Feb. 20. PMID: 29463557; PMCID: PMC7397497.
17. Blandin Knight S, Crosbie P A, Balata H, Chudziak J, Hussell T, Dive C. Progress and prospects of early detection in lung cancer. Open Biol. 2017 September; 7(9):170070. doi: 10.1098/rsob.170070. PMID: 28878044; PMCID: PMC5627048.
18. Dama E, Colangelo T, Fina E, Cremonesi M, Kallikourdis M, Veronesi G, Bianchi F. Biomarkers and Lung Cancer Early Detection: State of the Art. Cancers (Basel). 2021 Aug. 3; 13(15):3919. doi: 10.3390/cancers13153919. PMID: 34359818; PMCID: PMC8345487.
19. Harangus A, Berindan-Neagoe I, Todea D A, Simon I, Simon M. Noncoding RNAs and Liquid Biopsy in Lung Cancer: A Literature Review. Diagnostics (Basel). 2019 Dec. 9; 9(4):216. doi: 10.3390/diagnostics9040216. PMID: 31818027; PMCID: PMC6963838.
20. de Fraipont F, Gazzeri S, Cho W C, Eymin B. Circular RNAs and RNA Splice Variants as Biomarkers for Prognosis and Therapeutic Response in the Liquid Biopsies of Lung Cancer Patients. Front Genet. 2019 May 7; 10:390. doi: 10.3389/fgene.2019.00390. PMID: 31134126; PMCID: PMC6514155.
21. Zhong S, Golpon H, Zardo P, Borlak J. miRNAs in lung cancer. A systematic review identifies predictive and prognostic miRNA candidates for precision medicine in lung cancer. Transl Res. 2021 April; 230:164-196. doi: 10.1016/j.trsl.2020.11.012. Epub 2020 Nov. 28. PMID: 33253979.
22. Chen C C, Bai C H, Lee K Y, Chou Y T, Pan S T, Wang Y H. Evaluation of the diagnostic accuracy of bronchial brushing cytology in lung cancer: A meta-analysis. Cancer Cytopathol. 2021 September; 129(9):739-749. doi: 10.1002/cncy.22436. Epub 2021 Apr. 22. PMID: 33886162.
23. Pavel A B, Campbell J D, Liu G, Elashoff D, Dubinett S, Smith K, Whitney D, Lenburg M E, Spira A; AEGIS Study Team. Alterations in Bronchial Airway miRNA Expression for Lung Cancer Detection. Cancer Prev Res (Phila). 2017 November; 10(11):651-659. doi: 10.1158/1940-6207.CAPR-17-0098. Epub 2017 Sep. 6. PMID: 28877936; PMCID: PMC6758560.
24. Matthiesen R. M S-Based Biomarker Discovery in Bronchoalveolar Lavage Fluid for Lung Cancer. Proteomics Clin Appl. 2020 January; 14(1):e1900077. doi: 10.1002/prca.201900077. Epub 2019 Nov. 12. PMID: 31631581.
25. Engel E, Schmidt B, Carstensen T, Weickmann S, Jandrig B, Witt C, Fleischhacker M. Detection of tumor-specific mRNA in cell-free bronchial lavage supernatant in patients with lung cancer. Ann N Y Acad Sci. 2004 June; 1022:140-6. doi: 10.1196/annals.1318.023. PMID: 15251953.
26. Anglim P P, Alonzo T A, Laird-Offringa I A. DNA methylation-based biomarkers for early detection of non-small cell lung cancer: an update. Mol Cancer. 2008 Oct. 23; 7:81. doi: 10.1186/1476-4598-7-81. PMID: 18947422; PMCID: PMC2585582.
27. Schmidt B, Rehbein G, Fleischhacker M. Liquid Profiling in Lung Cancer—Quantification of Extracellular miRNAs in Bronchial Lavage. Adv Exp Med Biol. 2016; 924:33-37. doi: 10.1007/978-3-319-42044-8_7. PMID: 27753015.
28. Mutlu G M, Garey K W, Robbins R A, Danziger L H, Rubinstein I. Collection and analysis of exhaled breath condensate in humans. Am J Respir Crit Care Med. 2001 Sep. 1; 164(5):7317. doi: 10.1164/ajrccm.164.5.2101032. PMID: 11549524.
29. Rahimpour E, Khoubnasabjafari M, Jouyban-Gharamaleki V, Jouyban A. Non-volatile compounds in exhaled breath condensate: review of methodological aspects. Anal Bioanal Chem. 2018 October; 410(25):6411-6440. doi: 10.1007/s00216-018-1259-4. Epub 2018 Jul. 25. PMID: 30046867.
30. Papineni R S, Rosenthal F S. The size distribution of droplets in the exhaled breath of healthy human subjects. J Aerosol Med. 1997 Summer; 10(2):105-16. doi: 10.1089/jam.1997.10.105. PMID: 10168531.
31. Horvith I, Hunt J, Barnes P J, Alving K, Antczak A, Baraldi E, Becher G, van Beurden W J, Corradi M, Dekhuijzen R, Dweik R A, Dwyer T, Effros R, Erzurum S, Gaston B, Gessner C, Greening A, Ho L P, Hohlfeld J, Jöbsis Q, Laskowski D, Loukides S, Marlin D, Montuschi P, Olin A C, Redington A E, Reinhold P, van Rensen E L, Rubinstein I, Silkoff P, Toren K, Vass G, Vogelberg C, Wirtz H; ATS/ERS Task Force on Exhaled Breath Condensate. Exhaled breath condensate: methodological recommendations and unresolved questions. Eur Respir J. 2005 September; 26(3):523-48. doi: 10.1183/09031936.05.00029705. PMID: 16135737.
32. Boots A W, Bos L D, van der Schee M P, van Schooten F J, Sterk P J. Exhaled Molecular Fingerprinting in Diagnosis and Monitoring: Validating Volatile Promises. Trends Mol Med. 2015 October; 21(10):633-644. doi: 10.1016/j.molmed.2015.08.001. PMID: 26432020.
33. Winters B R, Pleil J D, Angrish M M, Stiegel M A, Risby T H, Madden M C. Standardization of the collection of exhaled breath condensate and exhaled breath aerosol using a feedback regulated sampling device. J Breath Res. 2017 Nov. 1; 11(4):047107. doi: 10.1088/1752-7163/aa8bbc. PMID: 28894051; PMCID: PMC5735826.
34. Wallace M A G, Pleil J D. Evolution of clinical and environmental health applications of exhaled breath research: Review of methods and instrumentation for gas-phase, condensate, and aerosols. Anal Chim Acta. 2018 Sep. 18; 1024:18-38. doi: 10.1016/j.aca.2018.01.069. Epub 2018 Feb. 9. PMID: 29776545; PMCID: PMC6082128.
35. Hunt J. Exhaled breath condensate: an overview. Immunol Allergy Clin North Am. 2007 November; 27(4):587-96; v. doi: 10.1016/j.iac.2007.09.001. PMID: 17996577; PMCID: PMC2170898.
36. Rahimpour E, Khoubnasabjafari M, Jouyban-Gharamaleki V, Jouyban A. Non-volatile compounds in exhaled breath condensate: review of methodological aspects. Anal Bioanal Chem. 2018 October; 410(25):6411-6440. doi: 10.1007/s00216-018-1259-4. Epub 2018 Jul. 25. PMID: 30046867.
37. Mazzatenta A, Pokorski M, Di Giulio C. Volatile organic compounds (VOCs) in exhaled breath as a marker of hypoxia in multiple chemical sensitivity. Physiol Rep. 2021 September; 9(18):e15034. doi: 10.14814/phy2.15034. PMID: 34536058; PMCID: PMC8449310.
38. Papineni R S, Rosenthal F S. The size distribution of droplets in the exhaled breath of healthy human subjects. J Aerosol Med. 1997 Summer; 10(2):105-16. doi: 10.1089/jam.1997.10.105. PMID: 10168531.
39. Horvith I, Hunt J, Barnes P J, Alving K, Antczak A, Baraldi E, Becher G, van Beurden W J, Corradi M, Dekhuijzen R, Dweik R A, Dwyer T, Effros R, Erzurum S, Gaston B, Gessner C, Greening A, Ho L P, Hohlfeld J, J6bsis Q, Laskowski D, Loukides S, Marlin D, Montuschi P, Olin A C, Redington A E, Reinhold P, van Rensen E L, Rubinstein I, Silkoff P, Toren K, Vass G, Vogelberg C, Wirtz H; ATS/ERS Task Force on Exhaled Breath Condensate. Exhaled breath condensate: methodological recommendations and unresolved questions. Eur Respir J. 2005 September; 26(3):523-48. doi: 10.1183/09031936.05.00029705. PMID: 16135737.
40. Powers K A, Dhamoon A S. Physiology, Pulmonary Ventilation and Perfusion. 2023 Jan. 23. In: StatPearls [Internet]. Treasure Island (FL): StatPearls Publishing; 2023 January-. PMID: 30969729.
41. Crystal R G, Randell S H, Engelhardt J F, Voynow J, Sunday M E. Airway epithelial cells: current concepts and challenges. Proc Am Thorac Soc. 2008 Sep. 15; 5(7):772-7. doi: 10.1513/pats.200805-041HR. PMID: 18757316; PMCID: PMC5820806.
42. Chang A, Ramsay P, Zhao B, Park M, Magdaleno S, Reardon M J, Welty S, DeMayo F J. Physiological regulation of uteroglobin/CCSP expression. Ann N Y Acad Sci. 2000; 923:181-92. doi: 10.1111/j.1749-6632.2000.tb05529.x. PMID: 11193756.
43. Whitsett J A, Wert S E, Weaver T E. Alveolar surfactant homeostasis and the pathogenesis of pulmonary disease. Annu Rev Med. 2010; 61:105-19. doi: 10.1146/annurev.med.60.041807.123500. PMID: 19824815; PMCID: PMC4127631.
44. Leibel S L, Winquist A, Tseu I, Wang J, Luo D, Shojaie S, Nathan N, Snyder E, Post M. Reversal of Surfactant Protein B Deficiency in Patient Specific Human Induced Pluripotent Stem Cell Derived Lung Organoids by Gene Therapy. Sci Rep. 2019 Sep. 17; 9(1):13450. doi: 10.1038/s41598-019-49696-8. PMID: 31530844; PMCID: PMC6748939.
45. Krygier A, Szmajda-Krygier D, Swiechowski R, Pietrzak J, Wosiak A, Wodzinski D, Balcerczak E. Molecular Pathogenesis of Fibrosis, Thrombosis and Surfactant Dysfunction in the Lungs of Severe COVID-19 Patients. Biomolecules. 2022 Dec. 10; 12(12):1845. doi: 10.3390/biom12121845. PMID: 36551272; PMCID: PMC9776352.
46. Dermer G B. Origin of bronchioloalveolar carcinoma and peripheral bronchial adenocarcinoma. Cancer. 1982 Mar. 1; 49(5):881-7. doi: 10.1002/1097-0142(19820301)49:5<881::aid-cncr2820490511>3.0.co; 2-7. PMID: 6277455.
47. Jackson E L, Willis N, Mercer K, Bronson R T, Crowley D, Montoya R, Jacks T, Tuveson D A. Analysis of lung tumor initiation and progression using conditional expression of oncogenic K-ras. Genes Dev. 2001 Dec. 15; 15(24):3243-8. doi: 10.1101/gad.943001. PMID: 11751630; PMCID: PMC312845.
48. Xu X, Rock J R, Lu Y, Futtner C, Schwab B, Guinney J, Hogan B L, Onaitis M W. Evidence for type II cells as cells of origin of K-Ras-induced distal lung adenocarcinoma. Proc Natl Acad Sci USA. 2012 Mar. 27; 109(13):4910-5. doi: 10.1073/pnas.1112499109. Epub 2012 Mar. 12. PMID: 22411819; PMCID: PMC3323959.
49. Xiao P, Chen J R, Zhou F, Lu C X, Yang Q, Tao G H, Tao Y J, Chen J L. Methylation of P16 in exhaled breath condensate for diagnosis of non-small cell lung cancer. Lung Cancer. 2014 January; 83(1):56-60. doi: 10.1016/j.lungcan.2013.09.008. Epub 2013 Sep. 25. PMID: 24268095.
50. Youssef O, Knuuttila A, Piirilä P, Böhling T, Sarhadi V, Knuutila S. Presence of cancer-associated mutations in exhaled breath condensates of healthy individuals by next generation sequencing. Oncotarget. 2017 Mar. 14; 8(11):18166-18176. doi: 10.18632/oncotarget.15233. PMID: 28199989; PMCID: PMC5392316.
51. Yang Ai S S, Hsu K, Herbert C, Cheng Z, Hunt J, Lewis C R, Thomas P S. Mitochondrial DNA mutations in exhaled breath condensate of patients with lung cancer. Respir Med. 2013 June; 107(6):911-8. doi: 10.1016/j.rmed.2013.02.007. Epub 2013 Mar. 16. PMID: 23507584.
52. Mehta A, Cordero J, Dobersch S, Romero-Olmedo A J, Savai R, Bodner J, Chao C M, Fink L, Guzmin-Diaz E, Singh I, Dobreva G, Rapp U R, Gunther S, Ilinskaya O N, Bellusci S, Dammann R H, Braun T, Seeger W, Gattenlahner S, Tresch A, Gunther A, Barreto G. Non-invasive lung cancer diagnosis by detection of GATA6 and NKX2-1 isoforms in exhaled breath condensate. EMBO Mol Med. 2016 Dec. 1; 8(12):1380-1389. doi: 10.15252/emmm.201606382. PMID: 27821429; PMCID: PMC5167131.
53. Shi M, Han W, Loudig O, Shah C D, Dobkin J B, Keller S, Sadoughi A, Zhu C, Siegel R E, Fernandez M K, DeLaRosa L, Patel D, Desai A, Siddiqui T, Gombar S, Suh Y, Wang T, Hosgood H D, Pradhan K, Ye K, Spivack S D. Initial development and testing of an exhaled microRNA detection strategy for lung cancer case-control discrimination. Sci Rep. 2023 Apr. 24; 13(1):6620. doi: 10.1038/s41598-023-33698-8.PMID: 37095155.
54. Pérez-Sánchez C, Barbarroja N, Pantaleão L C, López-Sánchez L M, Ozanne S E, Jurado-Gámez B, Aranda E, Lopez-Pedrera C, Rodríguez-Ariza A. Clinical Utility of microRNAs in Exhaled Breath Condensate as Biomarkers for Lung Cancer. J Pers Med. 2021 Feb. 9; 11(2):111. doi: 10.3390/jpm11020111. PMID: 33572343; PMCID: PMC7916163.
55. Xie H, Chen J, Lv X, Zhang L, Wu J, Ge X, Yang Q, Zhang D, Chen J. Clinical Value of Serum and Exhaled Breath Condensate miR-186 and IL-10 Levels in Non-Small Cell Lung Cancer. Technol Cancer Res Treat. 2020 January-December; 19:1533033820947490. doi: 10.1177/1533033820947490. PMID: 32851926; PMCID: PMC7457640.
56. Rai D, Pattnaik B, Bangaru S, Bhatraju N K, Tak J, Kashyap S, Verma U, Vadala R, Yadav G, Dhaliwal R S, Agrawal A, Guleria R, Mohan A. MicroRNAs in exhaled breath condensate: A pilot study of biomarker detection for lung cancer. Cancer Treat Res Commun. 2023; 35:100689. doi: 10.1016/j.ctarc.2023.100689. Epub 2023 Feb. 10. PMID: 36773435.
57. O'Brien J, Hayder H, Zayed Y, Peng C. Overview of MicroRNA Biogenesis, Mechanisms of Actions, and Circulation. Front Endocrinol (Lausanne). 2018 Aug. 3; 9:402. doi: 10.3389/fendo.2018.00402. PMID: 30123182; PMCID: PMC6085463.
58. Acunzo M, Romano G, Wernicke D, Croce C M. MicroRNA and cancer—a brief overview. Adv Biol Regul. 2015 January; 57:1-9. doi: 10.1016/j.jbior.2014.09.013. Epub 2014 Sep. 28. Erratum in: Adv Biol Regul. 2015 May; 58:53. PMID: 25294678.
59. Santos R M, Moreno C, Zhang W C. Non-Coding RNAs in Lung Tumor Initiation and Progression. Int J Mol Sci. 2020 Apr. 16; 21(8):2774. doi: 10.3390/ijms21082774. PMID: 32316322; PMCID: PMC7215285.
60. Yanaihara N, Caplen N, Bowman E, Seike M, Kumamoto K, Yi M, Stephens R M, Okamoto A, Yokota J, Tanaka T, Calin G A, Liu C G, Croce C M, Harris C C. Unique microRNA molecular profiles in lung cancer diagnosis and prognosis. Cancer Cell. 2006 March; 9(3):189-98. doi: 10.1016/j.ccr.2006.01.025. PMID: 16530703.
61. Asakura K, Kadota T, Matsuzaki J, Yoshida Y, Yamamoto Y, Nakagawa K, Takizawa S, Aoki Y, Nakamura E, Miura J, Sakamoto H, Kato K, Watanabe S I, Ochiya T. A miRNA-based diagnostic model predicts resectable lung cancer in humans with high accuracy. Commun Biol. 2020 Mar. 19; 3(1):134. doi: 10.1038/s42003-020-0863-y. PMID: 32193503; PMCID: PMC7081195.
62. Zhu X, Kudo M, Huang X, Sui H, Tian H, Croce C M, Cui R. Frontiers of MicroRNA Signature in Non-small Cell Lung Cancer. Front Cell Dev Biol. 2021 Apr. 7; 9:643942. doi: 10.3389/fcell.2021.643942. PMID: 33898432; PMCID: PMC8058364.
63. Cherchi R, Cusano R, Orrù S, Ferrari P A, Massidda M, Fotia G, De Matteis S, Cocco P. Next Generation Sequencing for miRNA Detection on the Exhaled Breath Condensate: A Pilot Study. Epigenet Insights. 2023 Apr. 2; 16:25168657231160985. doi: 10.1177/25168657231160985. PMID: 37025420; PMCID: PMC10070752.
64. Sinha A, Yadav A K, Chakraborty S, Kabra S K, Lodha R, Kumar M, Kulshreshtha A, Sethi T, Pandey R, Malik G, Laddha S, Mukhopadhyay A, Dash D, Ghosh B, Agrawal A. Exosome-enclosed microRNAs in exhaled breath hold potential for biomarker discovery in patients with pulmonary diseases. J Allergy Clin Immunol. 2013 July; 132(1):219-22. doi: 10.1016/j.jaci.2013.03.035. Epub 2013 May 14. PMID: 23683467.
65. Dobhal G, Datta A, Ayupova D, Teesdale-Spittle P, Goreham R V. Isolation, characterisation and detection of breath-derived extracellular vesicles. Sci Rep. 2020 Oct. 15; 10(1):17381. doi: 10.1038/s41598-020-73243-5. PMID: 33060613; PMCID: PMC7566616.
66. Lucchetti D, Santini G, Perelli L, Ricciardi-Tenore C, Colella F, Mores N, Macis G, Bush A, Sgambato A, Montuschi P. Detection and characterisation of extracellular vesicles in exhaled breath condensate and sputum of COPD and severe asthma patients. Eur Respir J. 2021 Aug. 5; 58(2):2003024. doi: 10.1183/13993003.03024-2020. PMID: 33795323.
67. Purghe B, Manfredi M, Ragnoli B, Baldanzi G, Malerba M. Exosomes in chronic respiratory diseases. Biomed Pharmacother. 2021 December; 144:112270. doi: 10.1016/j.biopha.2021.112270. Epub 2021 Oct. 19. PMID: 34678722.
68. van Niel G, D'Angelo G, Raposo G. Shedding light on the cell biology of extracellular vesicles. Nat Rev Mol Cell Biol. 2018 April; 19(4):213-228. doi: 10.1038/nrm.2017.125. Epub 2018 Jan. 17. PMID: 29339798.
69. Sheta M, Taha E A, Lu Y, Eguchi T. Extracellular Vesicles: New Classification and Tumor Immunosuppression. Biology (Basel). 2023 Jan. 10; 12(1):110. doi: 10.3390/biology12010110. PMID: 36671802; PMCID: PMC9856004.
70. O'Brien K, Breyne K, Ughetto S, Laurent L C, Breakefield X O. RNA delivery by extracellular vesicles in mammalian cells and its applications. Nat Rev Mol Cell Biol. 2020 October; 21(10):585-606. doi: 10.1038/s41580-020-0251-y. Epub 2020 May 26. PMID: 32457507; PMCID: PMC7249041.
71. Mills J, Capece M, Cocucci E, Tessari A, Palmieri D. Cancer-Derived Extracellular Vesicle-Associated MicroRNAs in Intercellular Communication: One Cell's Trash Is Another Cell's Treasure. Int J Mol Sci. 2019 Dec. 4; 20(24):6109. doi: 10.3390/ijms20246109. PMID: 31817101; PMCID: PMC6940802.
72. He X, Park S, Chen Y, Lee H. Extracellular Vesicle-Associated miRNAs as a Biomarker for Lung Cancer in Liquid Biopsy. Front Mol Biosci. 2021 Feb. 24; 8:630718. doi: 10.3389/fmolb.2021.630718. PMID: 33718435; PMCID: PMC7943919.
73. Mao S, Lu Z, Zheng S, Zhang H, Zhang G, Wang F, Huang J, Lei Y, Wang X, Liu C, Sun N, He J. Exosomal miR-141 promotes tumor angiogenesis via KLF12 in small cell lung cancer. J Exp Clin Cancer Res. 2020 Sep. 21; 39(1):193. doi: 10.1186/s13046-020-016801. PMID: 32958011; PMCID: PMC7504642.
74. He S, Li Z, Yu Y, Zeng Q, Cheng Y, Ji W, Xia W, Lu S. Exosomal miR-499a-5p promotes cell proliferation, migration and EMT via mTOR signaling pathway in lung adenocarcinoma. Exp Cell Res. 2019 Jun. 15; 379(2):203-213. doi: 10.1016/j.yexcr.2019.03.035. Epub 2019 Apr. 10. PMID: 30978341.
75. Fabbri M, Paone A, Calore F, Galli R, Gaudio E, Santhanam R, Lovat F, Fadda P, Mao C, Nuovo G J, Zanesi N, Crawford M, Ozer G H, Wernicke D, Alder H, Caligiuri M A, Nana-Sinkam P, Perrotti D, Croce C M. MicroRNAs bind to Toll-like receptors to induce prometastatic inflammatory response. Proc Natl Acad Sci USA. 2012 Jul. 31; 109(31):E2110-6. doi: 10.1073/pnas.1209414109. Epub 2012 Jul. 2. PMID: 22753494; PMCID: PMC3412003.
76. Zeng Z, Li Y, Pan Y, Lan X, Song F, Sun J, Zhou K, Liu X, Ren X, Wang F, Hu J, Zhu X, Yang W, Liao W, Li G, Ding Y, Liang L. Cancer-derived exosomal miR-25-3p promotes pre-metastatic niche formation by inducing vascular permeability and angiogenesis. Nat Commun. 2018 Dec. 19; 9(1):5395. doi: 10.1038/s41467-018-07810-w. PMID: 30568162; PMCID: PMC6300604.
77. Lucchetti D, Santini G, Perelli L, Ricciardi-Tenore C, Colella F, Mores N, Macis G, Bush A, Sgambato A, Montuschi P. Detection and characterisation of extracellular vesicles in exhaled breath condensate and sputum of COPD and severe asthma patients. Eur Respir J. 2021 Aug. 5; 58(2):2003024. doi: 10.1183/13993003.03024-2020. PMID: 33795323.
78. Dobhal G, Datta A, Ayupova D, Teesdale-Spittle P, Goreham R V. Isolation, characterisation and detection of breath-derived extracellular vesicles. Sci Rep. 2020 Oct. 15; 10(1):17381. doi: 10.1038/s41598-020-73243-5. PMID: 33060613; PMCID: PMC7566616.
79. An J, McDowell A, Kim Y K, Kim T B. Extracellular vesicle-derived microbiome obtained from exhaled breath condensate in patients with asthma. Ann Allergy Asthma Immunol. 2021 June; 126(6):729-731. doi: 10.1016/j.anai.2021.02.030. Epub 2021 Mar. 10. PMID: 33713805.
80. Mitchell M. I., Ben-Dov I. Z., Liu C., Ye K., Chow K., Kramer Y., Gangadharan A., Park S., Fitzgerald S., Ramnauth A., Perlin D. S., Donato M., Bhoy E., Manouchehri Doulabi E., Poulos M., Kamali-Moghaddam M., Loudig O. Extracellular Vesicle Capture by AnTibody of CHoice and Enzymatic Release (EV-CATCHER): A customizable purification assay designed for small-RNA biomarker identification and evaluation of circulating small-EVs. J Extracell Vesicles. 2021; 10(8):e12110. PMID: 34122779.
81. Loudig O, Wang T, Ye K, Lin J, Wang Y, Ramnauth A, Liu C, Stark A, Chitale D, Greenlee R, Multerer D, Honda S, Daida Y, Spencer Feigelson H, Glass A, Couch F J, Rohan T, Ben-Dov I Z. Evaluation and Adaptation of a Laboratory-Based cDNA Library Preparation Protocol for Retrospective Sequencing of Archived MicroRNAs from up to 35-Year-Old Clinical FFPE Specimens. Int J Mol Sci. 2017; 18(3). pii: E627. PMID: 28335433.
82. Farazi T A, Brown M, Morozov P, Ten Hoeve J J, Ben-Dov I Z, Hovestadt V, Hafner M, Renwick N, Mihailović A, Wessels L F, Tuschl T. Bioinformatic analysis of barcoded cDNA libraries for small RNA profiling by next-generation sequencing. Methods. 2012 October; 58(2):171-87. doi: 10.1016/j.ymeth.2012.07.020. Epub 2012 Jul. 23. PMID: 22836126; PMCID: PMC3597438.
83. Mazeh H, Deutch T, Karas A, Bogardus K A, Mizrahi I, Gur-Wahnon D, Ben-Dov I Z. Next-Generation Sequencing Identifies a Highly Accurate miRNA Panel That Distinguishes Well-Differentiated Thyroid Cancer from Benign Thyroid Nodules. Cancer Epidemiol Biomarkers Prev. 2018 August; 27(8):858-863. doi: 10.1158/1055-9965.EPI-18-0055. Epub 2018 Jul. 26. PMID: 30049841.
84. Cizmar P, Yuana Y. Detection and Characterization of Extracellular Vesicles by Transmission and Cryo-Transmission Electron Microscopy. Methods Mol Biol. 2017; 1660:221-232. doi: 10.1007/978-1-4939-7253-1_18. PMID: 28828660.
85. Wu F, Yin Z, Yang L, Fan J, Xu J, Jin Y, Yu J, Zhang D, Yang G. Smoking Induced Extracellular Vesicles Release and Their Distinct Properties in Non-Small Cell Lung Cancer. J Cancer. 2019 Jun. 9; 10(15):3435-3443. doi: 10.7150/jca.30425. PMID: 31293647; PMCID: PMC6603414.
86. Théry C, Witwer K W, Aikawa E, Alcaraz M J, Anderson J D, Andriantsitohaina R, Antoniou A, Arab T, Archer F, Atkin-Smith G K, Ayre D C, Bach J M, Bachurski D, Baharvand H, Balaj L, Baldacchino S, Bauer N N, Baxter A A, Bebawy M, Beckham C, Bedina Zavec A, Benmoussa A, Berardi A C, Bergese P, Bielska E, Blenkiron C, Bobis-Wozowicz S, Boilard E, Boireau W, Bongiovanni A, Borras F E, Bosch S, Boulanger C M, Breakefield X, Breglio A M, Brennan M Á, Brigstock D R, Brisson A, Broekman M L, Bromberg J F, Bryl-G0recka P, Buch S, Buck A H, Burger D, Busatto S, Buschmann D, Bussolati B, Buzás El, Byrd J B, Camussi G, Carter D R, Caruso S, Chamley L W, Chang Y T, Chen C, Chen S, Cheng L, Chin A R, Clayton A, Clerici S P, Cocks A, Cocucci E, Coffey R J, Cordeiro-da-Silva A, Couch Y, Coumans F A, Coyle B, Crescitelli R, Criado M F, D'Souza-Schorey C, Das S, Datta Chaudhuri A, de Candia P, De Santana E F, De Wever O, Del Portillo H A, Demaret T, Deville S, Devitt A, Dhondt B, Di Vizio D, Dieterich L C, Dolo V, Dominguez Rubio A P, Dominici M, Dourado M R, Driedonks T A, Duarte F V, Duncan H M, Eichenberger R M, Ekström K, El Andaloussi S, Elie-Caille C, Erdbrügger U, Falc0n-Pérez J M, Fatima F, Fish J E, Flores-Bellver M, Försönits A, Frelet-Barrand A, Fricke F, Fuhrmann G, Gabrielsson S, Gámez-Valero A, Gardiner C, Gärtner K, Gaudin R, Gho Y S, Giebel B, Gilbert C, Gimona Giusti I, Goberdhan D C, Görgens A, Gorski S M, Greening D W, Gross J C, Gualerzi A, Gupta G N, Gustafson D, Handberg A, Haraszti R A, Harrison P, Hegyesi H, Hendrix A, Hill A F, Hochberg F H, Hoffmann K F, Holder B, Holthofer H, Hosseinkhani B, Hu G, Huang Y, Huber V, Hunt S, Ibrahim A G, Ikezu T, Inal J M, Isin M, Ivanova A, Jackson H K, Jacobsen S, Jay S M, Jayachandran M, Jenster G, Jiang L, Johnson S M, Jones J C, Jong A, Jovanovic-Talisman T, Jung S, Kalluri R, Kano S I, Kaur S, Kawamura Y, Keller E T, Khamari D, Khomyakova E, Khvorova A, Kierulf P, Kim K P, Kislinger T, Klingeborn M, Klinke D J 2nd, Kornek M, Kosanović M M, Kovács Á F, Krämer-Albers E M, Krasemann S, Krause M, Kurochkin I V, Kusuma G D, Kuypers S, Laitinen S, Langevin S M, Languino L R, Lannigan J, Lässer C, Laurent L C, Lavieu G, Lázaro-Ibíñez E, Le Lay S, Lee M S, Lee Y X F, Lemos D S, Lenassi M, Leszczynska A, Li I T, Liao K, Libregts S F, Ligeti E, Lim R, Lim S K, Linē A, Linnemannstöns K, Llorente A, Lombard C A, Lorenowicz M J, Lörincz Á M, Lötvall J, Lovett J, Lowry M C, Loyer X, Lu Q, Lukomska B, Lunavat T R, Maas S L, Malhi H, Marcilla A, Mariani J, Mariscal J, Martens-Uzunova E S, Martin-Jaular L, Martinez M C, Martins V R, Mathieu M, Mathivanan S, Maugeri M, McGinnis L K, McVey M J, Meckes D G Jr, Meehan K L, Mertens I, Minciacchi V R, Möller A, Møller Jørgensen M, Morales-Kastresana A, Morhayim J, Mullier F, Muraca M, Musante L, Mussack V, Muth D C, Myburgh K H, Najrana T, Nawaz M, Nazarenko I, Nejsum P, Neri C, Neri T, Nieuwland R, Nimrichter L, Nolan J P, Nolte-'t Hoen E N, Noren Hooten N, O'Driscoll L, O'Grady T, O'Loghlen A, Ochiya T, Olivier M, Ortiz A, Ortiz L A, Osteikoetxea X, Østergaard O, Ostrowski M, Park J, Pegtel D M, Peinado H, Perut F, Pfaffl M W, Phinney D G, Pieters B C, Pink R C, Pisetsky D S, Pogge von Strandmann E, Polakovicova I, Poon I K, Powell B H, Prada I, Pulliam L, Quesenberry P, Radeghieri A, Raffai R L, Raimondo S, Rak J, Ramirez Raposo G, Rayyan M S, Regev-Rudzki N, Ricklefs F L, Robbins P D, Roberts D D, Rodrigues S C, Rohde E, Rome S, Rouschop K M, Rughetti A, Russell A E, Saá P, Sahoo S, Salas-Huenuleo E, Sánchez C, Saugstad J A, Saul M J, Schiffelers R M, Schneider R, Schøyen T H, Scott A, Shahaj E, Sharma S, Shatnyeva O, Shekari F, Shelke G V, Shetty A K, Shiba K, Siljander P R, Silva A M, Skowronek A, Snyder O L 2nd, Soares R P, Sódar B W, Soekmadji C, Sotillo J, Stahl P D, Stoorvogel W, Stott S L, Strasser E F, Swift S, Tahara H, Tewari M, Timms K, Tiwari S, Tixeira R, Tkach M, Toh W S, Tomasini R, Torrecilhas A C, Tosar J P, Toxavidis V, Urbanelli L, Vader P, van Balkom B W, van der Grein S G, Van Deun J, van Herwijnen M J, Van Keuren-Jensen K, van Niel G, van Royen M E, van Wijnen A J, Vasconcelos M H, Vechetti I J Jr, Veit T D, Vella L J, Velot É, Verweij F J, Vestad B, Viñas J L, Visnovitz T, Vukman K V, Wahlgren J, Watson D C, Wauben M H, Weaver A, Webber J P, Weber V, Wehman A M, Weiss D J, Welsh J A, Wendt S, Wheelock A M, Wiener Z, Witte L, Wolfram J, Xagorari A, Xander P, Xu J, Yan X, Yáñez-Mó M, Yin H, Yuana Y, Zappulli V, Zarubova J, Žėkas V, Zhang J Y, Zhao Z, Zheng L, Zheutlin A R, Zickler A M, Zimmermann P, Zivkovic A M, Zocco D, Zuba-Surma E K. Minimal information for studies of extracellular vesicles 2018 (MISEV2018): a position statement of the International Society for Extracellular Vesicles and update of the MISEV2014 guidelines. J Extracell Vesicles. 2018 Nov. 23; 7(1):1535750. doi: 10.1080/20013078.2018.1535750. PMID: 30637094; PMCID: PMC6322352.
87. Rowbotham S P, Kim C F. Diverse cells at the origin of lung adenocarcinoma. Proc Natl Acad Sci USA. 2014 Apr. 1; 111(13):4745-6. doi: 10.1073/pnas.1401955111. Epub 2014 Mar. 20. PMID: 24707043; PMCID: PMC3977312.
88. 87-Lin C, Song H, Huang C, Yao E, Gacayan R, Xu S M, Chuang P T. Alveolar type II cells possess the capability of initiating lung tumor development. PLoS One. 2012; 7(12):e53817. doi: 10.1371/journal.pone.0053817. Epub 2012 Dec. 20. PMID: 23285300; PMCID: PMC3527621.
89. Sainz de Aja J, Dost A F M, Kim C F. Alveolar progenitor cells and the origin of lung cancer. J Intern Med. 2021 May; 289(5):629-635. doi: 10.1111/joim.13201. Epub 2020 Dec. 19. PMID: 33340175; PMCID: PMC8604037.
90. Broeckaert F, Bernard A. Clara cell secretory protein (CC16): characteristics and perspectives as lung peripheral biomarker. Clin Exp Allergy. 2000 April; 30(4):469-75. doi: 10.1046/j.1365-2222.2000.00760.x. PMID: 10718843.
91. Hayashida S, Harrod K S, Whitsett J A. Regulation and function of CCSP during pulmonary Pseudomonas aeruginosa infection in vivo. Am J Physiol Lung Cell Mol Physiol. 2000 September; 279(3):L452-9. doi: 10.1152/ajplung.2000.279.3.L452. PMID: 10956619.
92. Wang X Y, Keefe K M, Jensen-Taubman S M, Yang D, Yan K, Linnoila R I. Novel method for isolation of murine clara cell secretory protein-expressing cells with traces of stemness. PLoS One. 2012; 7(8):e43008. doi: 10.1371/journal.pone.0043008. Epub 2012 Aug. 16. PMID: 22916196; PMCID: PMC3420884.
93. Wong A P, Keating A, Waddell T K. Airway regeneration: the role of the Clara cell secretory protein and the cells that express it. Cytotherapy. 2009; 11(6):676-87. doi: 10.3109/14653240903313974. PMID: 19878054.
94. Rokicki W, Rokicki M, Wojtacha J, Dżeljijli A. The role and importance of club cells (Clara cells) in the pathogenesis of some respiratory diseases. Kardiochir Torakochirurgia Pol. 2016 March; 13(1):26-30. doi: 10.5114/kitp.2016.58961. Epub 2016 Mar. 30. PMID: 27212975; PMCID: PMC4860431.
95. Weaver T E, Whitsett J A. Function and regulation of expression of pulmonary surfactant-associated proteins. Biochem J. 1991 Jan. 15; 273(Pt 2)(Pt 2):249-64. doi: 10.1042/bj2730249. PMID: 1991023; PMCID: PMC1149839.
96. Mulugeta S, Beers M F. Surfactant protein C: its unique properties and emerging immunomodulatory role in the lung. Microbes Infect. 2006 July; 8(8):2317-23. doi: 10.1016/j.micinf.2006.04.009. Epub 2006 May 30. PMID: 16782390.
97. Dickens J A, Rutherford E N, Abreu S, Chambers J E, Ellis M O, van Schadewijk A, Hiemstra P S, Marciniak S J. Novel insights into surfactant protein C trafficking revealed through the study of a pathogenic mutant. Eur Respir J. 2022 Jan. 27; 59(1):2100267. doi: 10.1183/13993003.00267-2021. PMID: 34049951; PMCID: PMC8792467.
98. Nureki S I, Tomer Y, Venosa A, Katzen J, Russo S J, Jamil S, Barrett M, Nguyen V, Kopp M, Mulugeta S, Beers M F. Expression of mutant Sftpc in murine alveolar epithelia drives spontaneous lung fibrosis. J Clin Invest. 2018 Aug. 31; 128(9):4008-4024. doi: 10.1172/JC199287. Epub 2018 Aug. 13. PMID: 29920187; PMCID: PMC6118576.
99. Choudhary I, Vo T, Paudel K, Wen X, Gupta R, Kesimer M, Patial S, Saini Y. Vesicular and extravesicular protein analyses from the airspaces of ozone-exposed mice revealed signatures associated with mucoinflammatory lung disease. Sci Rep. 2021 Dec. 1; 11(1):23203. doi: 10.1038/s41598-021-02256-5. PMID: 34853335; PMCID: PMC8636509.
100. Sitaraman S, Martin E P, Na C L, Zhao S, Green J, Deshmukh H, Perl A T, Bridges J P, Xu Y, Weaver T E. Surfactant protein C mutation links postnatal type 2 cell dysfunction to adult disease. JCI Insight. 2021 Jul. 22; 6(14):e142501. doi: 10.1172/jci.insight.142501. PMID: 34138759; PMCID: PMC8410047.
101. Lam S M, Zhang C, Wang Z, Ni Z, Zhang S, Yang S, Huang X, Mo L, Li J, Lee B, Mei M, Huang L, Shi M, Xu Z, Meng F P, Cao W J, Zhou M J, Shi L, Chua G H, Li B, Cao J, Wang J, Bao S, Wang Y, Song J W, Zhang F, Wang F S, Shui G. A multi-omics investigation of the composition and function of extracellular vesicles along the temporal trajectory of COVID-19. Nat Metab. 2021 July; 3(7):909-922. doi: 10.1038/s42255-021-00425-4. Epub 2021 Jun. 22. PMID: 34158670.
102. Sutherland K D, Berns A. Cell of origin of lung cancer. Mol Oncol. 2010 October; 4(5):397-403. doi: 10.1016/j.molonc.2010.05.002. Epub 2010 Jun. 8. PMID: 20594926; PMCID: PMC5527931.
103. Mainardi S, Mijimolle N, Francoz S, Vicente-Duenas C, Sinchez-Garcia I, Barbacid M. Identification of cancer initiating cells in K-Ras driven lung adenocarcinoma. Proc Natl Acad Sci USA. 2014 Jan. 7; 111(1):255-60. doi: 10.1073/pnas.1320383110. Epub 2013 Dec. 23. PMID: 24367082; PMCID: PMC3890787.
104. Sainz de Aja J, Dost A F M, Kim C F. Alveolar progenitor cells and the origin of lung cancer. J Intern Med. 2021 May; 289(5):629-635. doi: 10.1111/joim.13201. Epub 2020 Dec. 19. PMID: 33340175; PMCID: PMC8604037.
105. El-Shennawy L, Hoffmann A D, Dashzeveg N K, McAndrews K M, Mehl P J, Cornish D, Yu Z, Tokars V L, Nicolaescu V, Tomatsidou A, Mao C, Felicelli C J, Tsai C F, Ostiguin C, Jia Y, Li L, Furlong K, Wysocki J, Luo X, Ruivo C F, Batlle D, Hope T J, Shen Y, Chae Y K, Zhang H, LeBleu V S, Shi T, Swaminathan S, Luo Y, Missiakas D, Randall G C, Demonbreun A R, Ison M G, Kalluri R, Fang D, Liu H. Circulating ACE2-expressing extracellular vesicles block broad strains of SARS-CoV-2. Nat Commun. 2022 Jan. 20; 13(1):405. doi: 10.1038/s41467021-27893-2. PMID: 35058437; PMCID: PMC8776790.
106. Osborne J K, Minna J D. Lung Cancer Cell of Origin: Controversy and Clinical Translational Implications. Cancer Res. 2022 Mar. 15; 82(6):972-973. doi: 10.1158/0008-5472.CAN-22-0301. PMID: 35288734.
107. Smith R V, Schlecht N F, Childs G, Prystowsky M B, Belbin T J. Pilot study of mucosal genetic differences in early smokers and nonsmokers. Laryngoscope. 2006 August; 116(8):1375-9. doi: 10.1097/01.mlg.0000228133.08067.f8. PMID: 16885739.
108. Gower A C, Steiling K, Brothers J F 2nd, Lenburg M E, Spira A. Transcriptomic studies of the airway field of injury associated with smoking-related lung disease. Proc Am Thorac Soc. 2011 May; 8(2):173-9. doi: 10.1513/pats.201011-066M S. PMID: 21543797; PMCID: PMC3159071.
109. Spira A, Beane J, Shah V, Liu G, Schembri F, Yang X, Palma J, Brody J S. Effects of cigarette smoke on the human airway epithelial cell transcriptome. Proc Natl Acad Sci USA. 2004 Jul. 6; 101(27):10143-8. doi: 10.1073/pnas.0401422101. Epub 2004 Jun. 21. PMID: 15210990; PMCID: PMC454179.
110. Liu F, Mao Q, Zhu S, Qiu J. MicroRNA-155-5p promotes cell proliferation and invasion in lung squamous cell carcinoma through negative regulation of fibroblast growth factor 9 expression. J Thorac Dis. 2021 June; 13(6):3669-3679. doi: 10.21037/jtd-21-882. PMID: 34277059; PMCID: PMC8264708.
111. Xu S, Shi L. High expression of miR-155 and miR-21 in the recurrence or metastasis of non-small cell lung cancer. Oncol Lett. 2019 July; 18(1):758-763. doi: 10.3892/ol.2019.10337. Epub 2019 May 8. PMID: 31289551; PMCID: PMC6539534.
112. Xue X, Liu Y, Wang Y, Meng M, Wang K, Zang X, Zhao S, Sun X, Cui L, Pan L, Liu S. MiR-21 and MiR-155 promote non-small cell lung cancer progression by downregulating SOCS1, SOCS6, and PTEN. Oncotarget. 2016 Dec. 20; 7(51):84508-84519. doi: 10.18632/oncotarget.13022. PMID: 27811366; PMCID: PMC5356677.
113. Ren X Y, Han Y D, Lin Q. Long non-coding RNA MIR155HG knockdown suppresses cell proliferation, migration and invasion in NSCLC by upregulating TP53INP1 directly targeted by miR-155-3p and miR-155-5p. Eur Rev Med Pharmacol Sci. 2020 May; 24(9):4822-4835. doi: 10.26355/eurrev_202005_21171. PMID: 32432745.
114. Patierno S R, Manyak M J, Fernandez P M, Baker A, Weeraratna A T, Chou D S, Szlyk G, Geib K S, Walsh C, Patteras J. Uteroglobin: a potential novel tumor suppressor and molecular therapeutic for prostate cancer. Clin Prostate Cancer. 2002 September; 1(2):118-24. doi: 10.3816/cgc.2002.n.014. PMID: 15046703.
115. Cioppi F, Simi L, Luciani P, Petraglia F, Susini T, Cobellis L, Serio M, Maggi M, Peri A. Expression of uteroglobin and matrix metalloproteinase-9 genes in endometrial cancer: relationship to estrogen and progesterone receptor status. Oncol Rep. 2004 February; 11(2):427-33. PMID: 14719079.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A non-invasive method for early detection of deep lung pathology in a subject at risk of the deep lung pathology comprising
(a) sampling deep lung tissue of the subject at risk of the deep lung pathology by isolating micro RNA (riRNA) cargo of lung tissue origin in exhaled extracellular vesicles (EVs) purified from exhaled breath condensates (EBCs) of the subject;
(b) purifying lung-specific exhaled extracellular vesicles in exhaled breath condensate by antibody capture of the lung-specific exhaled EVs and
(c) detecting the pathology by comparing an miRNA profile of the exhaled extracellular vesicles of lung tissue origin purified from the subject to the profile of exhaled extracellular vesicles purified from exhaled breath condensates of a healthy subject,
wherein the early detection leads to early treatment of the deep lung pathology and can lead to an improved health outcome for the subject.

2. The method according to claim 1, wherein the miRNA cargo comprises tissue-specific surface proteins derived from terminal bronchioles and alveoli.

3. The method according to claim 2, wherein the tissue-specific surface proteins derived from terminal bronchioles and alveoli detected on a surface of exhaled EVs comprise club cell secretory protein (CCSP), type 2 pneumocyte marker surfactant protein C (SFTPC) or both.

4. The method according to claim 1, wherein the EVs are purified from exhaled breath condensate by antibody capture of the lung specific exhaled EVs in the exhaled breath condensates.

5. The method according to claim 1, wherein the method for purifying lung specific exhaled extracellular vesicles in exhaled breach condensate by antibody capture of the lung-specific exhaled EVs comprises
(i) activating an antibody with a dibenzocyclo-octyl (DBCO)-ester to form a DBCO-modified antibody;
(ii) coupling the DBCO-modified antibody to a DNA linker by click chemistry,
(iii) binding the antibody-DNA linker conjugates to streptavidin coated well plates pretreated with RNAse A;
(iv) releasing the purified populations of deep lung-specific EVs from the streptavidin-coated well plates enzymatically by uracil glycosylase; and
(v) eluting the purified population of EVs from each antibody complex.

6. The method according to claim 5, wherein
(1) the antibody is a monoclonal antibody raised against CCSP;
(2) the antibody is a monoclonal antibody raised against SFTPC; or
(3) both.

7. The method according to claim 1, wherein profiles of the miRNA cargo isolated from the exhaled breath condensates by antibody capture correlate with profiles of miRNA purified from bronchoalveolar lavage fluid obtained from the subject.

8. The method according to claim 1, wherein the deep lung pathology comprises a lung dysfunction due to smoking.

9. The method according to claim 1, wherein the deep lung pathology comprises a lung dysfunction due to asthma.

10. The method according to claim 1, wherein the deep lung pathology comprises a lung dysfunction due to a lung cancer.
